(19) United States
(12) Patent Application Publication
(10) Pub. No.: US 2003/0176454 A1
(43) Pub. Date: Sep. 18, 2003

(54) N-coating heterocyclic compounds

(76) Inventors: Akira Yamada, Osaka-shi, Osaka (JP); Glen Spears, Osaka-shi, Osaka (JP); Hisashi Hayashida, Osaka-shi, Osaka (JP); Masaki Tomishima, Osaka-shi, Osaka (JP); Kiyotaka Ito, Osaka-shi, Osaka (JP); Masashi Imanishi, Osaka-shi, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/258,582

(22) PCT Filed: May 14, 2001
(86) PCT No.: PCT/JP01/04002

(30) Foreign Application Priority Data
May 15, 2000 (AU) PQ7501
Dec. 7, 2000 (AU) PR1955

(51) Int. Cl. C07D 221/18; C07D 215/16; C07D 209/04

(52) U.S. Cl. 514/284; 514/410; 514/411; 514/290; 514/311; 514/412; 546/61; 546/79; 546/153; 548/420; 548/427; 548/452

(57) ABSTRACT

A compound of the formula (I): wherein A is a hydrogen atom, an optionally substituted, unsaturated, N-containing heterocyclic group or a group of the formula (a): wherein R is an optionally substituted aryl group or an optionally substituted heterocyclic group; M is —(CH₂)n-, —(CH₂)n-O—(CH₂)m-or —(CH₂)n-NH—(CH₂)m-, wherein n and m are independently 0, 1 or 2; Q is an optionally substituted cycloalkylene group, an optionally substituted arylene group or an optionally substituted divalent heterocyclic group; and the moiety of the formula (b): is an optionally substituted, unsaturated, mono-, di-, tri- or tetra-cyclic, N-containing heterocyclic group which may contain additional hetero atom(s) selected from the group consisting of nitrogen, oxygen and sulfur atoms as the ring member(s), its prodrug or a pharmaceutically acceptable salt thereof.

### TECHNICAL FIELD

The present invention relates to novel N-containing heterocyclic compounds and salts thereof. More particularly, it relates to novel N-containing heterocyclic compounds and salts thereof which have pharmacological activities such as 5-hydroxytryptamine (5-HT) antagonism and the like.

Said compounds and their salts are useful as a 5-HT antagonist for treating or preventing central nervous system (CNS) disorders such as anxiety, depression, obsessive compulsive disorders, migraine, anorexia, Alzheimer's disease, sleep disorders, bulimia, panic attacks, withdrawal from drug abuse (e.g., with cocaine, ethanol, nicotine and benzodiazepines), schizophrenia, and also disorders associated with spinal trauma and/or head injury such as hydrocephalus in human beings and animals.

### BACKGROUND OF ART

With regard to the state of the art in this field, many N-containing heterocyclic compounds have been synthesized. For example, the following fused pyrimidine compound having 5-HT_{2c} antagonism is disclosed in WO97/12880.

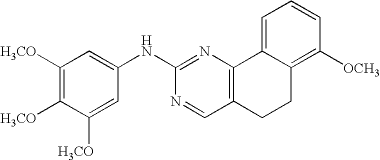

### DISCLOSURE OF INVENTION

This invention relates to a compound of the formula (I):

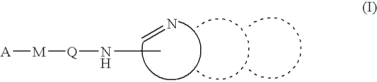

wherein A is a hydrogen atom, an optionally substituted, unsaturated, N-containing heterocyclic group or a group of the formula (a):

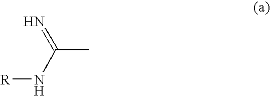

wherein R is an optionally substituted aryl group or an optionally substituted heterocyclic group;

M is —(CH₂)ₙ—, —(CH₂)ₙ—O—(CH₂)ₘ— or —(CH₂)ₙ—NH—(CH₂)ₘ—, wherein n and m are independently 0, 1 or 2;

Q is an optionally substituted cycloalkylene group, an optionally substituted arylene group or an optionally substituted, divalent heterocyclic group; and the moiety of the formula (b):

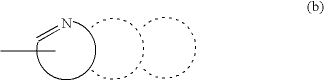

is an optionally substituted, unsaturated, mono-, di-, tri- or tetra-cyclic, N-containing heterocyclic group which may contain additional hetero atom(s) selected from the group consisting of nitrogen, oxygen and sulfur atoms as the ring member(s),

its prodrug or a pharmaceutically acceptable salt thereof.

In the above and subsequent descriptions of the present specification, suitable examples and illustrations of the various definitions which the present invention includes within the scope are explained in detail in the following.

Suitable unsaturated, N-containing heterocyclic group for A may be an unsaturated, 5 or 10-membered, mono- or di-cyclic heteromonocyclic group containing 1 to 4 nitrogen atoms, for example, pyrrolyl, imidazolyl, imidazolinyl, pyrazolyl, pyrazolinyl, pyridyl, pyrimidinyl, pyrazinyl, pyridazinyl, triazolyl [e.g., 4H-1,2,4-triazolyl, 1H-1,2,3-triazolyl or 2H-1,2,3-triazolyl], tetrazolyl [e.g., 1H-tetrazolyl or 2H-tetrazolyl], benzopyrrolyl, benzimidazolyl, benzopyrazolyl, benzotriazolyl, quinolyl, isoquinolyl, phthalazinyl, indolizinyl, isoindolyl, indolyl, naphthyridinyl, quinoxalinyl, quinazolinyl, cinnolinyl, imidazopyridyl, 1H-indazolyl or the like.

The heterocyclic group for A may be optionally substituted with one or more lower alkyl groups and/or hydroxy(lower)alkyl groups.

Suitable aryl group for R may be an aromatic hydrocarbon residue having 6 to 12 carbon atoms such as phenyl, naphthyl or the like, and said aryl group may be optionally substituted with one or more halogen atoms.

Suitable heterocyclic group for R may be an unsaturated, 5- to 6-membered heterocyclic group containing one or more hetero atoms selected from nitrogen, sulfur and oxygen atoms, for example, pyrrolyl, pyridyl, furyl, pyranyl, thienyl, thiopyranyl or the like.

These aryl group and heterocyclic group for R may be optionally substituted with one or more halogen atoms.

Suitable cysloalkylene moiety in the optionally substituted cycloalkylene group for Q may be a 4- to 8-membered cycloalkylene such as cyclobutylene, cyclopentylene, cyclohexylene, cycloheptylene or cyclooctylene.

Suitable arylene moiety in the optionally substituted arylene group for Q is phenylene group such as 1,2-phenylene, 1,3-phenylene or 1,4-phenylene or naphthalenediyl group such as naphthalene-1,2-diyl.

Suitable heterocyclic moiety in the optionally substituted, divalent heterocyclic group for Q is 6-membered, divalent heterocyclic group containing 1 to 2 nitrogen atoms, such as pyridinediyl group (e.g., pyridine-2,3-diyl, pyridine-2,4-diyl or pyridine-2,5-diyl), or pyrimidinediyl group (e.g., pyrimidine-2,4-diyl, pyrimidine-2,5-diyl or pyrimidine-2,6-diyl).

These cycloalkylene, arylene and divalent heterocyclic groups for Q may be optionally substituted with one or more halogen atoms, lower alkyl, lower alkoxy and/or halo(lower)alkyl groups.

Suitable heterocyclic moiety in the optionally substituted, unsaturated, mono-, di-, tri- or tetra-cyclic, N-containing heterocyclic group which may contain additional hetero atom(s) selected from the group consisting of nitrogen, oxygen and sulfur atoms as the ring member(s) which is represented by the formula (b) may include:

(1) unsaturated, 5- to 6-membered, monocyclic groups containing nitrogen atom(s) or nitrogen and sulfur atoms, more particularly 1 to 2 nitrogen atoms, or 1 to 2 nitrogen and 1 sulfur atoms, for example, pyrrolyl, imidazolyl, pyrazolyl, pyridyl, pyrimidinyl, pyridazinyl, pyrazinyl, thiazolyl or thiadiazolyl;

(2) unsaturated, 9- to 10-membered, dicyclic group containing nitrogen atom(s), or nitrogen and oxygen atoms, or nitrogen and sulfur atoms, more particularly 1 to 3 nitrogen atoms, or 1 to 2 nitrogen and 1 oxygen atom, or 1 to 2 nitrogen and 1 sulfur atoms, for example, indolyl, isoindolyl, indolizinyl, indazolyl, quinalizinyl, quinolyl, isoquinolyl, phthalazinyl, naphthyridinyl, quinoxalinyl, quinazolinyl, cinnolinyl, benzimidazolyl, benzotriazolyl, imidazopyridyl, benzoxazolyl, benzisoxazolyl, benzothiazolyl, benzisothiazolyl or tetrahydrocycloheptapyrimidinyl;

(3) unsaturated, 12- to 15-membered, tri-cyclic group containing nitrogen atom(s), or nitrogen and oxygen atoms, or nitrogen and sulfur atoms, more particularly 1 to 4 nitrogen atoms, or 1 to 3 nitrogen and 1 to 2 oxygen atoms, or 1 to 3 nitrogen and 1 to 2 sulfur atoms, for example, dihydrobenzoquinazolinyl, indenopyrimidinyl, carbazolyl, carbolinyl, phenanthridinyl, acridinyl, perimidinyl, phenanthrolinyl, phenazinyl, phenoxazinyl, dihydrobenzoxepinoisoxazolyl, dihydrobenzoxepinopyrimidinyl, phenothiazinyl, dihydrothienoquinazolinyl, dihydronaphthothiazolyl or indenothiazolyl; and

(4) unsaturated, 15- to 17-membered tetra-cyclic group containing nitrogen atoms, more particularly 1 to 3 nitrogen atoms, for eample, pyrazinocarbazolyl, pyridocarbazolyl or indenophthalazinyl.

These mono-, di-, tri or tetra-cyclic group may be optionally substituted with one or more halogen atoms, lower alkyl, lower alkoxy, halo(lower)alkyl, aryl, aryloxy, arylamino and/or 5-membered heterocyclic group, among which the aryl group may further be substituted with one or more halogen atoms, hydroxy, lower alkyl and/or lower alkoxy groups.

Among the above heterocyclic groups, specific examples of the unsaturated, N-containing heterocyclic group for A are:

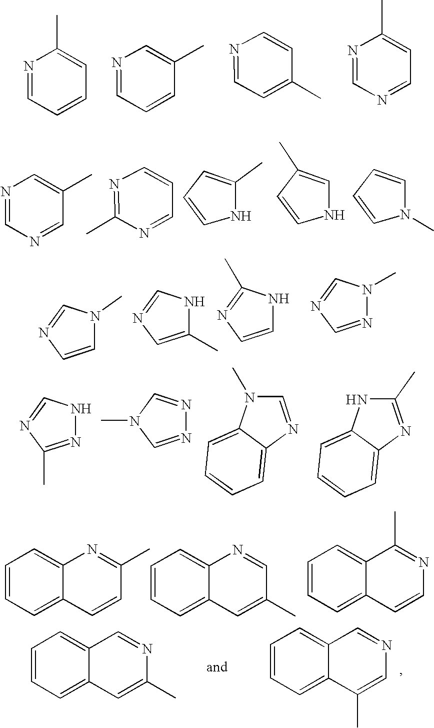

specific examples of the heterocyclic group for R are:

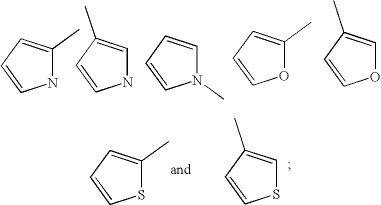

and specific examples of the unsaturated, mono- di-, tri- or tetra-cyclic, N-containing heterocyclic group represented by the formula (b) which may contain additional hetero atom(s) selected from the group consisting of nitrogen, oxygen and sulfur atoms as the ring member(s) are:

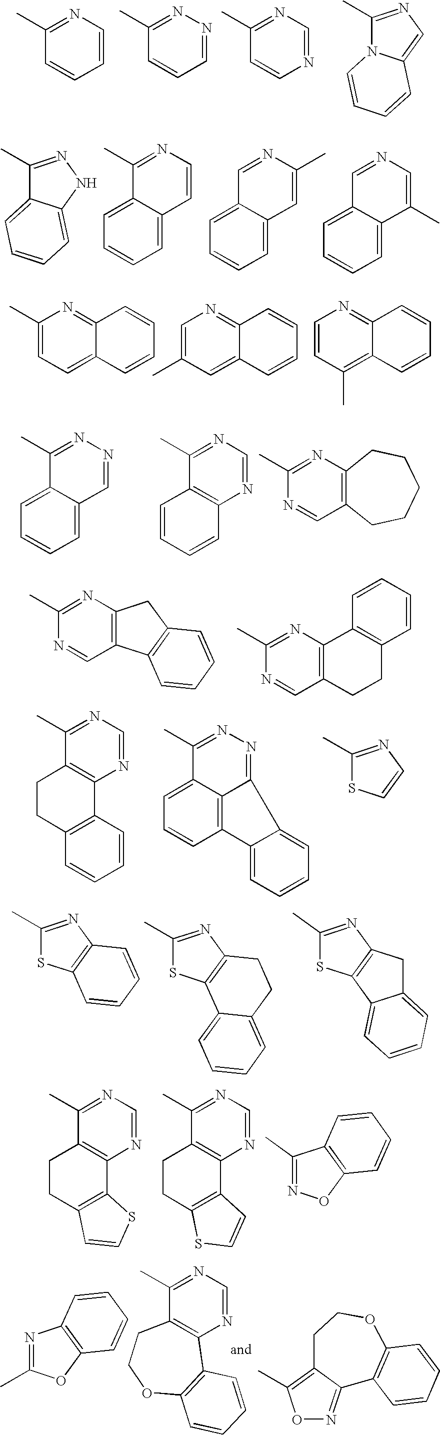

Preferable examples of the above-mentioned substituents on the aryl, cycloalkylene, arylene and/or heterocyclic group are illustrated in the following.

Preferable halogen atom is fluorine, chlorine, bromine or iodine.

The term “lower” is intended to mean 1 to 6 carbon atoms, preferably 1 to 4 carbon atoms, unless otherwise indicated.

Preferable lower alkyl group is a straight or branched one having 1 to 6 carbon atoms such as methyl, ethyl, propyl, isopropyl, butyl, isobutyl, sec-butyl, tert-butyl, pentyl, isopentyl, hexyl or the like.

The hydroxy(lower)alkyl group is a lower alkyl group substituted with one or more hydroxy groups. Preferred examples of the hydroxy(lower)alkyl group include hydroxymethyl, 2-hydroxyethyl, 3-hydroxypropyl, 4-hydroxybutyl, 5-hydroxypentyl, 6-hydroxyhexyl, 2,3-dihydroxypropyl and the like.

The halo(lower)alkyl group is a lower alkyl group substituted with one or more halogen atoms, in which the lower alkyl moiety and the halogen atom are the same as exemplified in the above, respectively. Preferred examples of the halo(lower)alkyl group include fluoromethyl, difluoromethyl, trifluoromethyl, chloromethyl, dichloromethyl, trichloromethyl, bromomethyl, iodomethyl, fluoroethyl, 2,2,2-trifluoroethyl, chloroethyl, 2,2,2-trichloroethyl, bromoethyl, iodoethyl, chloropropyl, bromopropyl, chlorobutyl, bromobutyl, chloropentyl, bromopentyl, chlorohexyl, bromohexyl and the like.

Preferable lower alkoxy group is a straight or branched one having 1 to 6 carbon atoms such as methoxy, ethoxy, propoxy, isopropoxy, n-butoxy, isobutoxy, tert-butoxy, pentyloxy, hexyloxy or the like.

Preferable examples of the aryl groups include phenyl and naphthyl. Examples of the aryl group which may be further substituted with halogen, alkoxy and/or hydroxy include 2-chlorophenyl, 2-bromophenyl, 2-fluorophenyl, 3-chlorophenyl, 4-chlorophenyl, 4-bromophenyl, 4-fluorophenyl, 2,4-dichlorophenyl, 5-chloro-2-methoxyphenyl, 5-chloro-2-hydroxyphenyl, 2-methoxyphenyl and the like.

Preferable examples of the aryloxy group include phenoxy and naphthoxy.

Preferable examples of the arylamino group include anilino and naphthylamino.

Preferable examples of the 5-membered heterocyclic group as a substituent include pyrrolyl, imidazolyl, furyl, thienyl, oxazolyl, ozadiazolyl, thiazolyl and thiadiazolyl.

The position(s) of the above substituent(s) on the aryl, cycloalkylene, arylene or heterocyclic group is(are) optional.

Examples of the substituted, unsaturated, N-containing heterocyclic groups for A are:

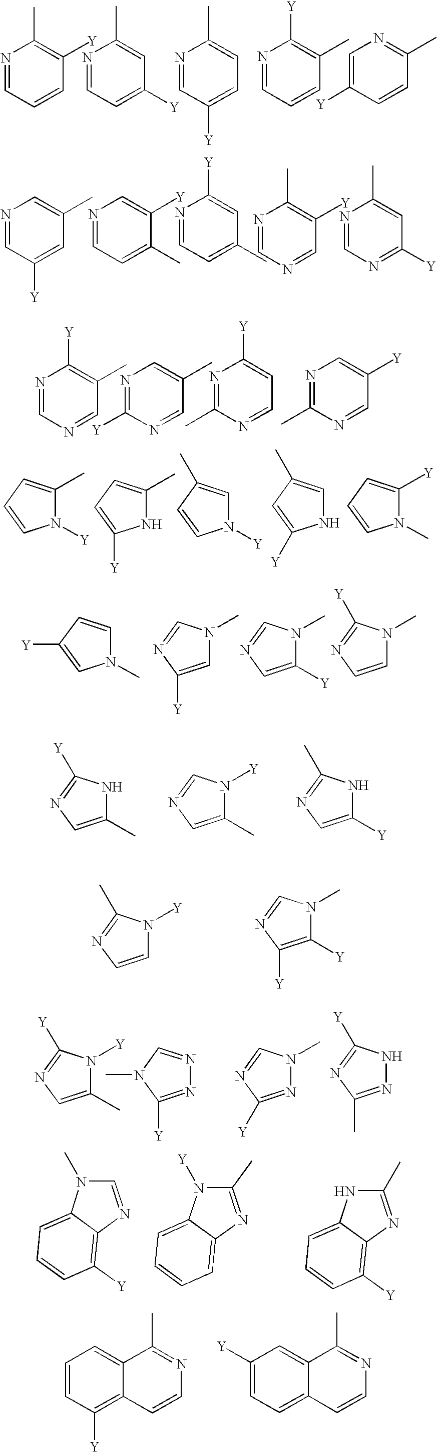

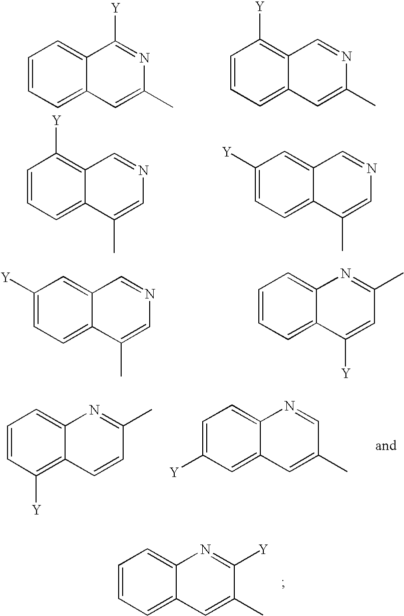

examples of the substituted aryl groups for R are:

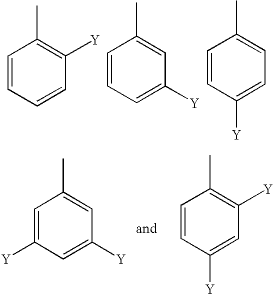

examples of the substituted heterocyclic groups for R are:

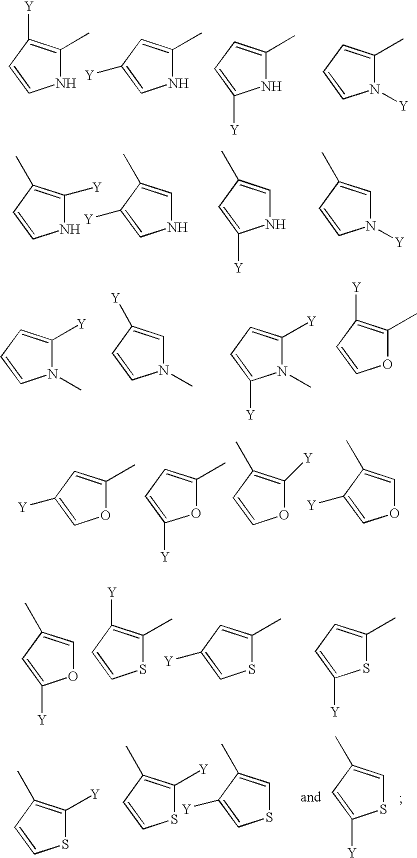

examples of the substituted phenylene groups for Q are:

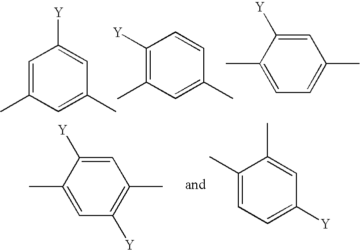

examples of the substituted pyridinediyl groups for Q are:

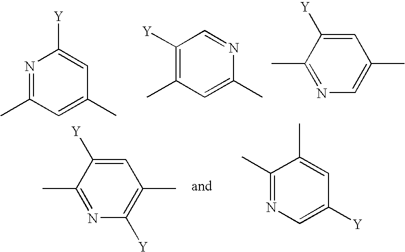

examples of the substituted pyrimidinediyl groups for Q are:

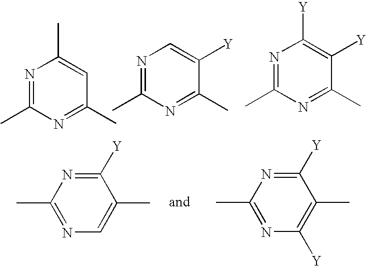

examples of the substituted cycloalkylene groups for Q are:

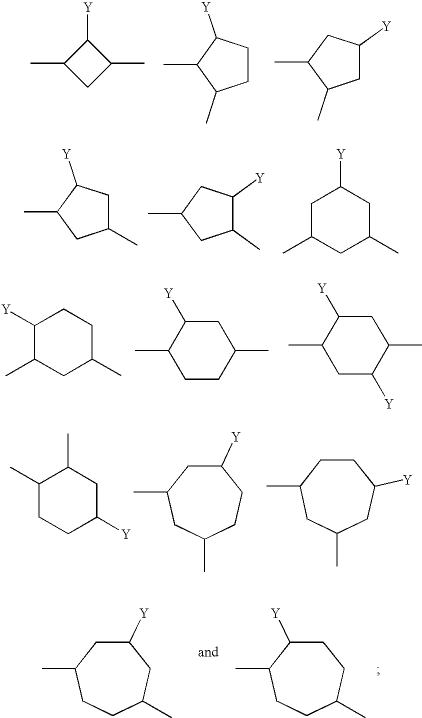

examples of the substituted, unsaturated, mono-, di-, tri- or tetra-cyclic, N-containing heterocyclic groups which may contain additional hetero atom(s) selected from the group consisting of nitrogen, oxygen and sulfur atoms as the ring member(s) for the moiety of the formula (b) are;

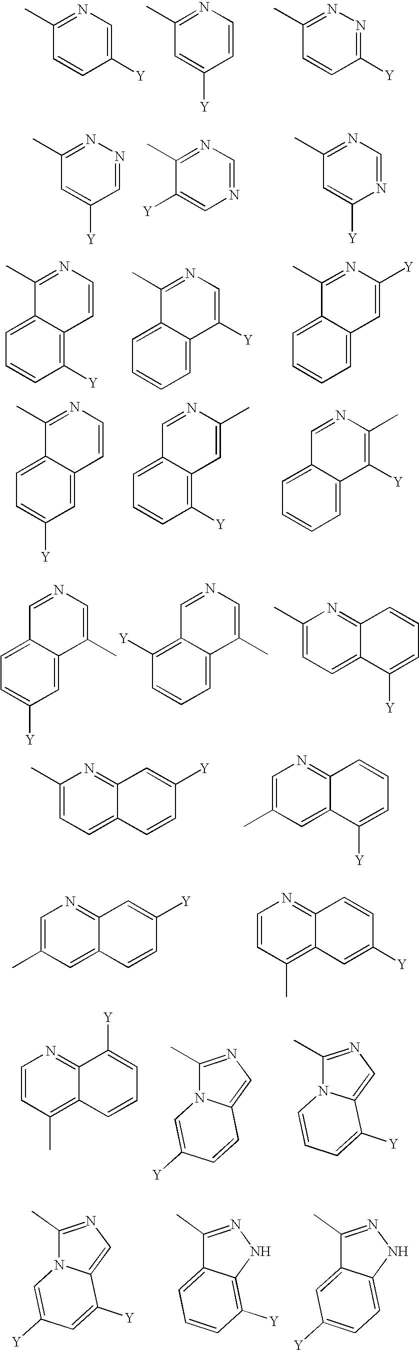

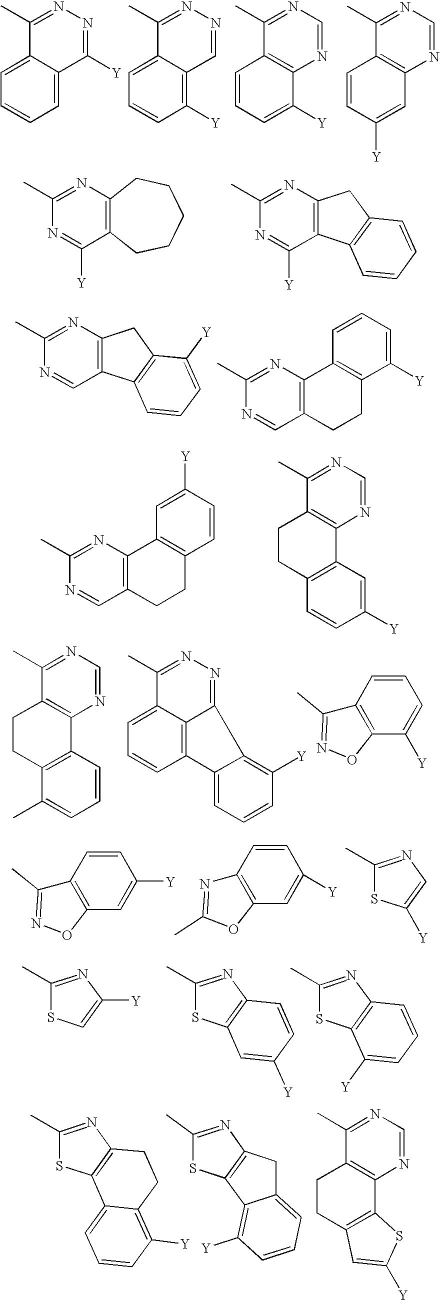

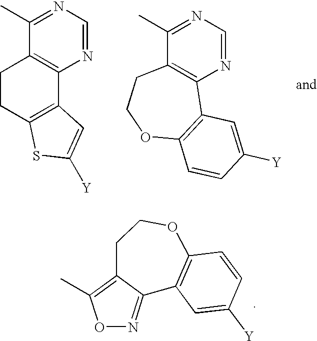

Preferred embodiments of the compounds (I) are those represented by the formula (I), wherein

A is an optionally substituted, unsaturated, 5-membered, N-containing heterocyclic group,

M is a group of —(CH₂)ₙ— in which n is 0,

Q is an optionally substituted arylene group, and

the moiety of the formula (b) is an optionally substituted, unsaturated tricyclic, N-containing heterocyclic group containing 2 nitrogen atoms.

More preferred embodiments of the compounds (I) are those represented by the formula (I), wherein

A is an unsaturated, 5-membered, N-containing heterocyclic group substituted with lower alkyl,

M is a group of —(CH₂)ₙ— in which n is 0,

Q is arylene group, and

the moiety of the formula (b) is an optionally substituted, unsaturated, tricyclic heterocyclic group containing 2 nitrogen atoms.

Most preferred embodiments of the compounds (I) are those represented by the formula (I), wherein

A is imidazolyl group substituted with lower alky,

M is a group of —(CH₂)ₙ— in which n is 0,

Q is phenylene group, and

the moiety of formula (b) is 5,6-dihydrobenzo[h]quinazolinyl group which may be substituted with a halogen atom.

Specifically, the preferred embodiments are as follows:

N-[3-(4,5-dimethyl-1H-imidazol-1-yl)phenyl]-9-fluoro-5,6-dihydrobenzo[h]quinazolin-4-amine,

9-Fluoro-N-[3-(4-methyl-1H-imidazol-1-yl)phenyl]-5,6-dihydrobenzo[h]quinazolin-4-amine,

9-Fluoro-N-[3-(1,2-dimethyl-1H-imidazol-5-yl)phenyl]-5,6-dihydrobenzo[h]quinazolin-4-amine,

N-[3-(4-methyl-1H-imidazol-1-yl)phenyl]-5,6-dihydrobenzo[h]quinazolin-4-amine hydrochloride,

N-[3-(4-methyl-1H-imidazol-1-yl)phenyl]-5,6-dihydrobenzo[h]quinazolin-4-amine dihydrochloride,

N-[3-(4-methyl-1H-imidazol-1-yl)phenyl]-5,6-dihydrobenzo[h]quinazolin-4-amine methanesulfonate,

N-[3-(4-methyl-1H-imidazol-1-yl)phenyl]-5,6-dihydrobenzo[h]quinazolin-4-amine dimethanesulfonate,

N-[3-(1,2-dimethyl-1H-imidazol-5-yl)phenyl]-5,6-dihydrobenzo[h]quinazolin-4-amine,

N-[3-(4,5-dimethyl-1H-imidazol-1-yl)phenyl]-5,6-dihydrobenzo[h]quinazolin-4-amine or

N-[3-(4-methyl-1H-imidazol-1-yl)phenyl]-5,6-dihydrobenzo[h]quinazolin-4-amine.

Suitable pharmaceutically acceptable salts may include salts with inorganic bases, for example, alkali metals (e.g. sodium or potassium), alkaline earth metals (e.g. calcium or magnesium), ammonium; salts with organic bases, for example, organic amines (e.g. triethylamine, pyridine, picoline, ethanolamine, triethanolamine, dicyclohexylamine, or N,N′-dibenzylethylenediamine); inorganic acid addition salts (e.g. hydrochloride, hydrobromide, hydriodide, sulfate or phosphate); organic carboxylic or sulfonic acid addition salts (e.g. formate, acetate, trifluoroacetate, maleate, tartrate, methanesulfonate, benzenesulfonate or p-toluenesulfonate); salts with basic or acidic amino acids (e.g. arginine, aspartate or glutamate); and the like, and preferable example thereof is the acid addition salts.

According to the present invention, the compounds (I) and their salts can be prepared by the following processes. In the Processes, the moiety of the formula (b):

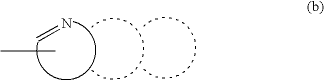

is represented by D for convenient sake.

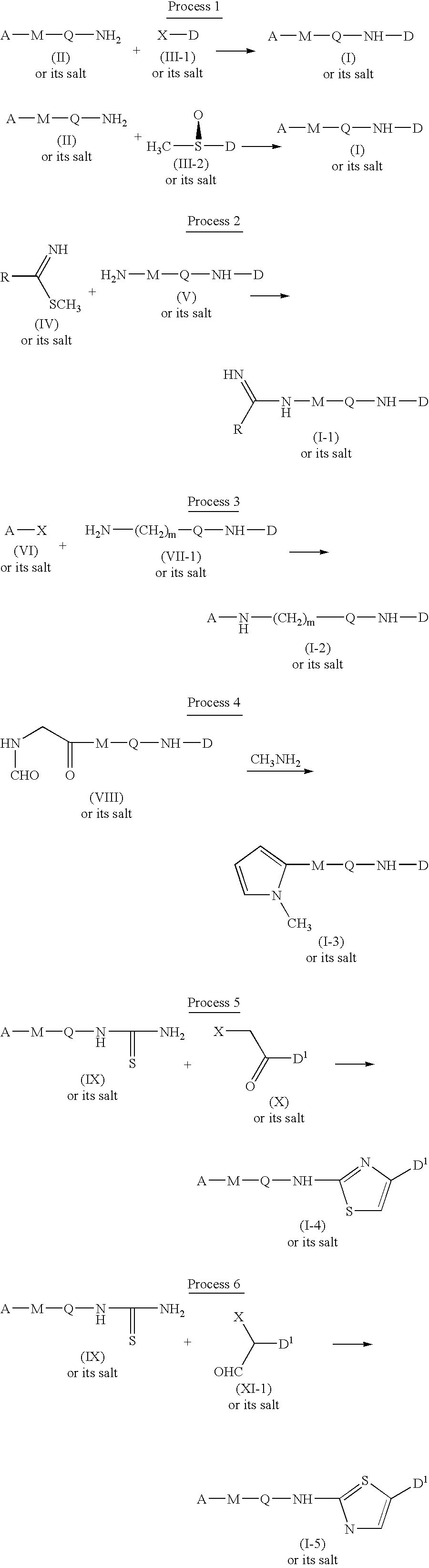

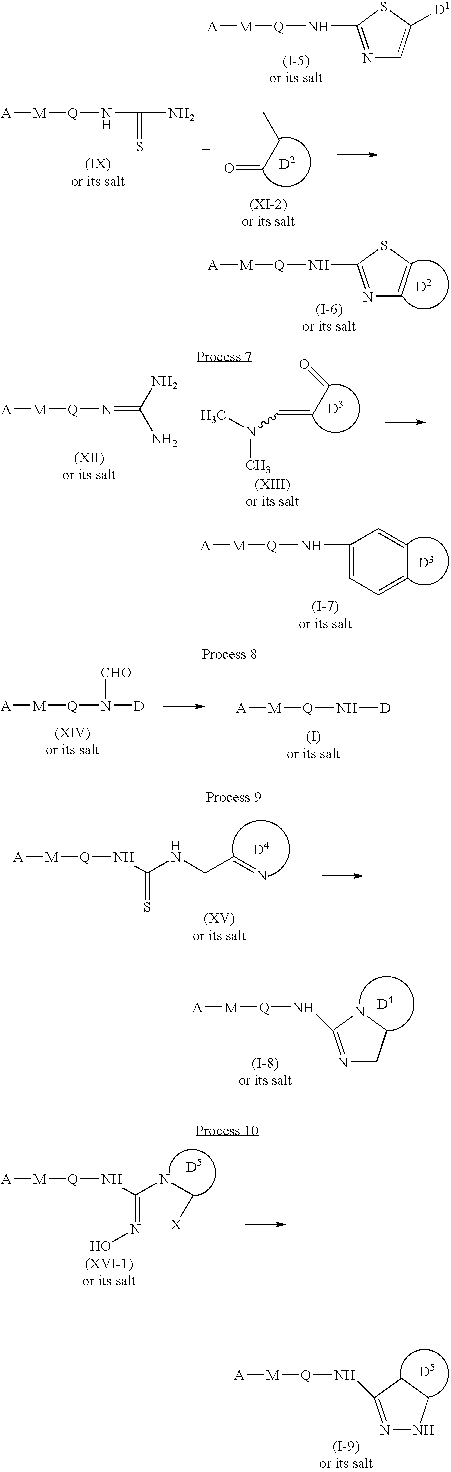

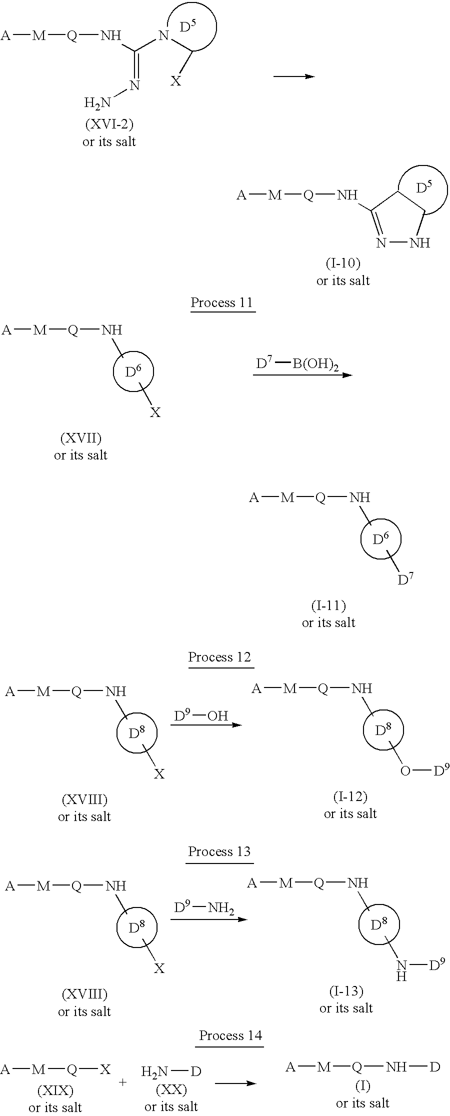

wherein A, M, Q and R are each as defined above, and X is a halogen atom.

Some of the starting compounds are novel and can be prepared by the following processes.

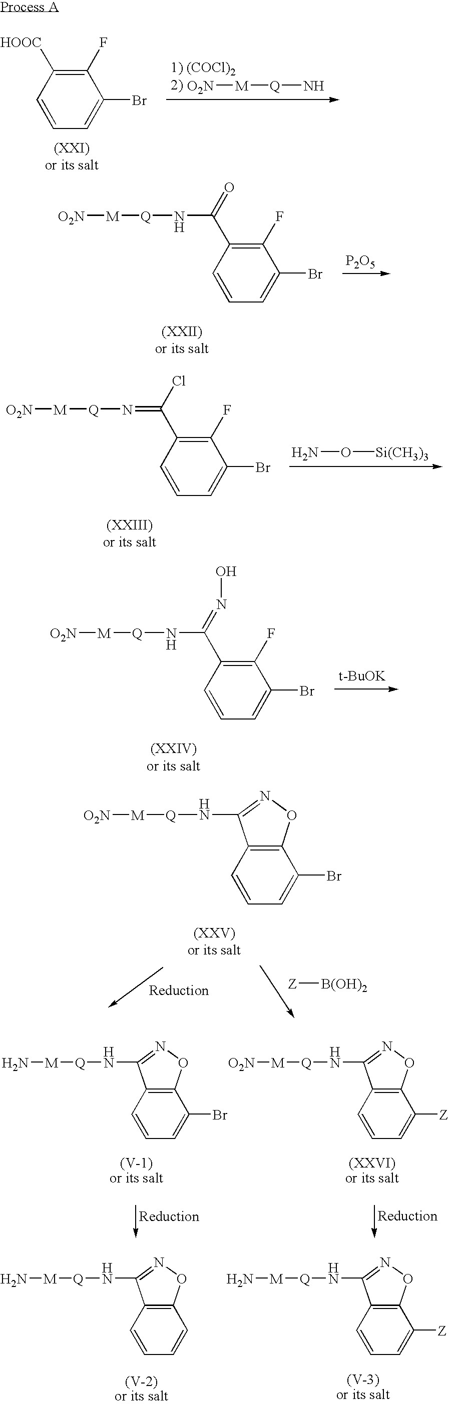

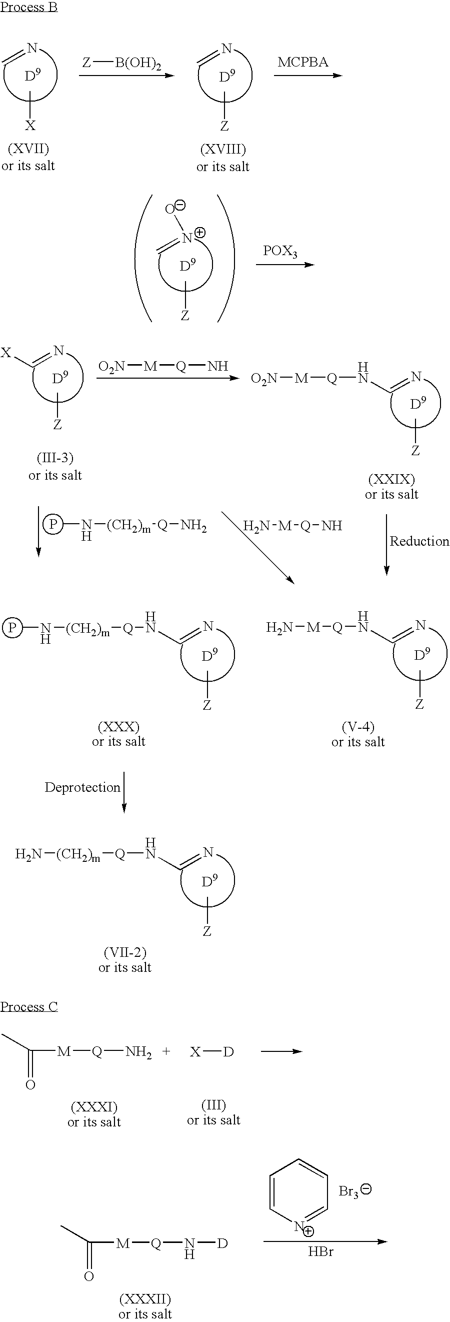

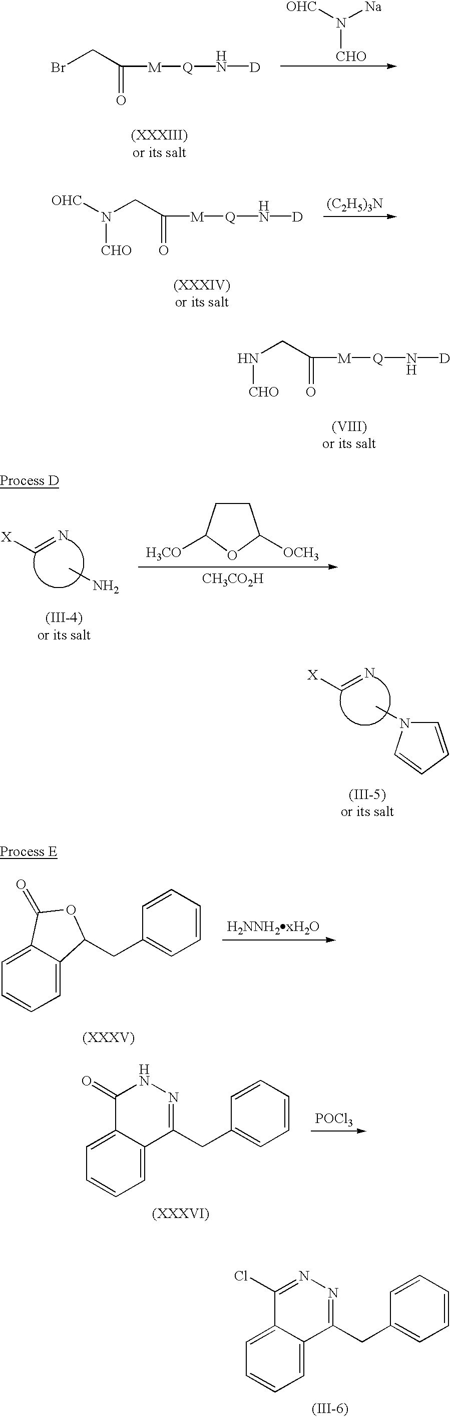

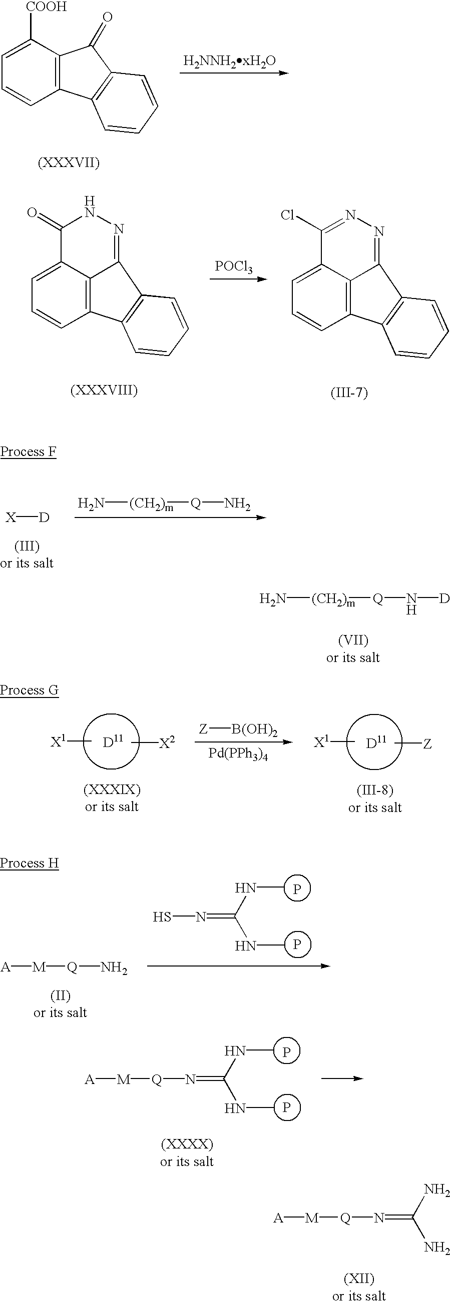

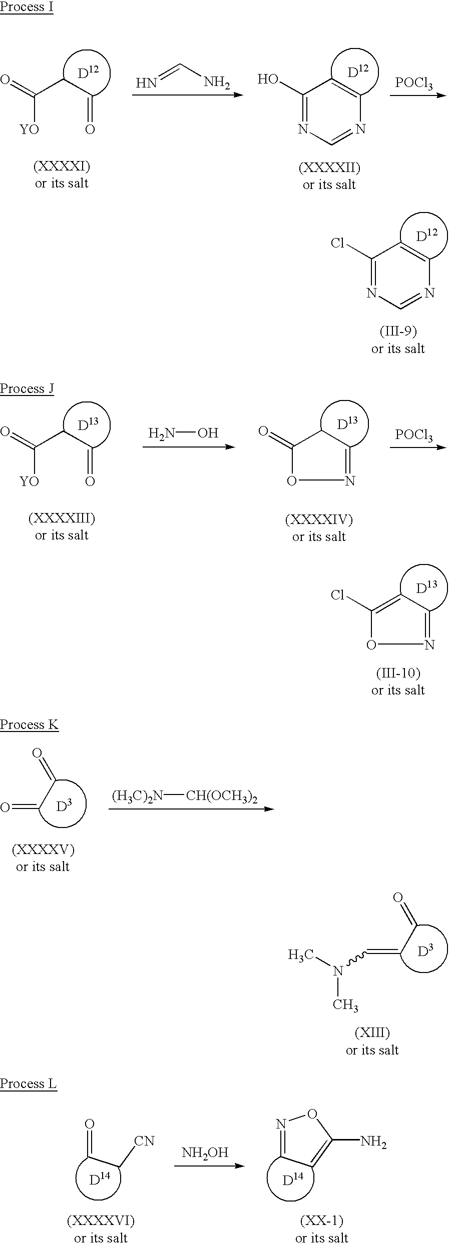

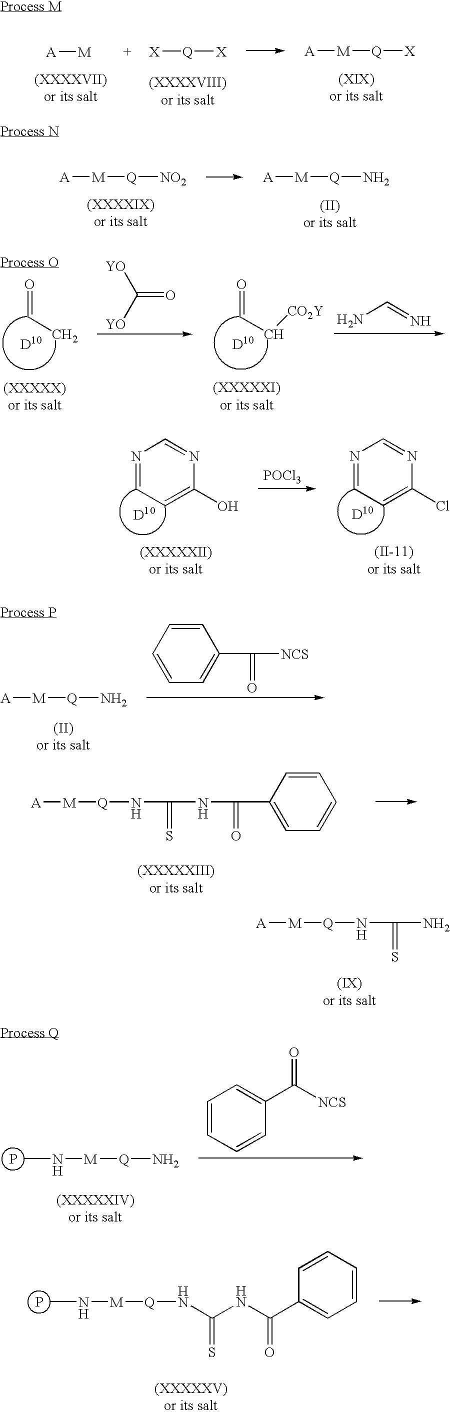

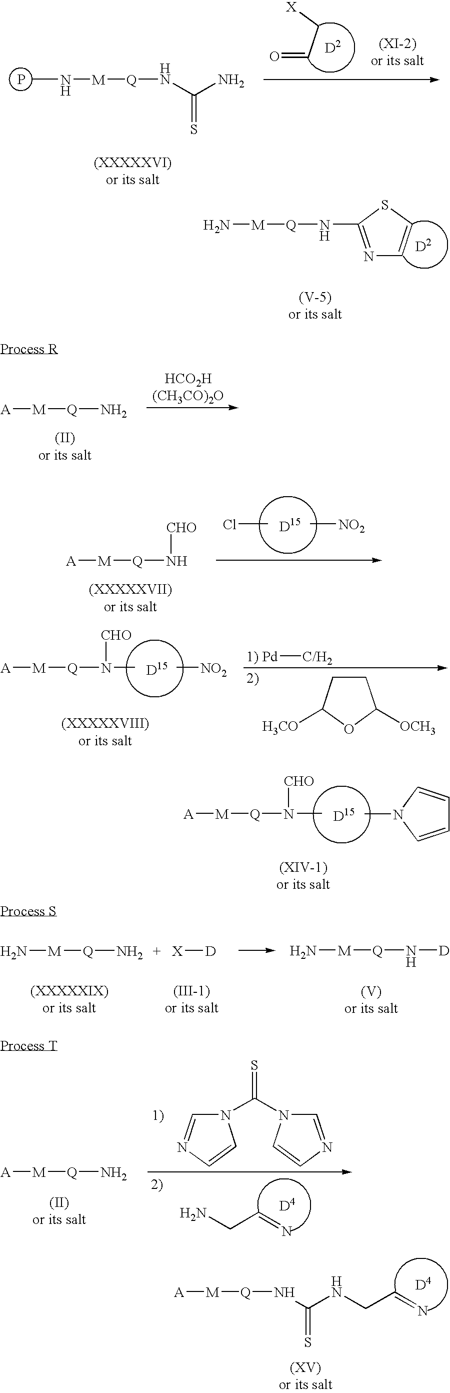

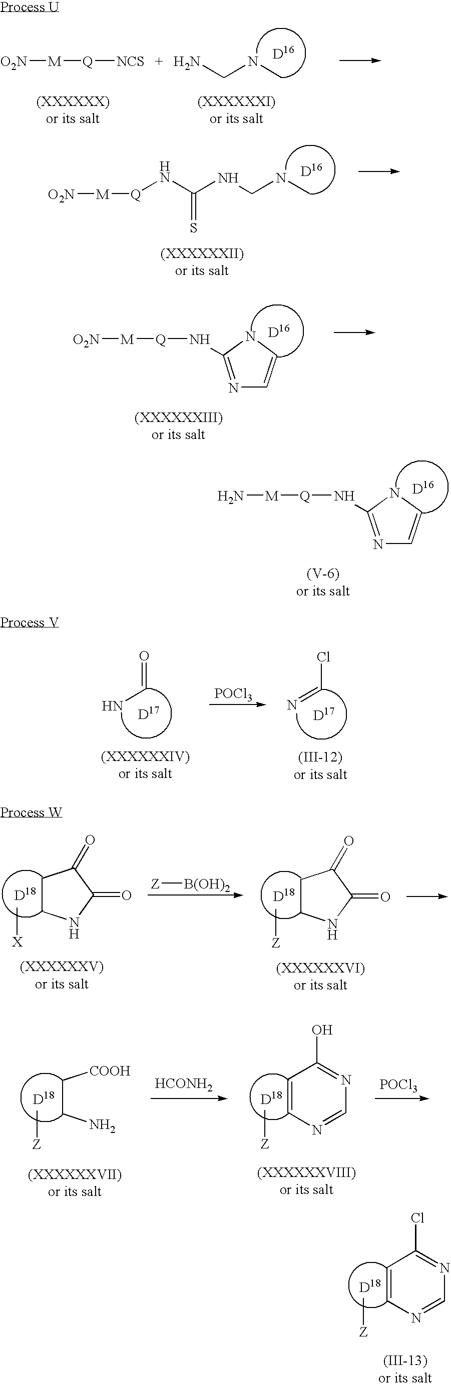

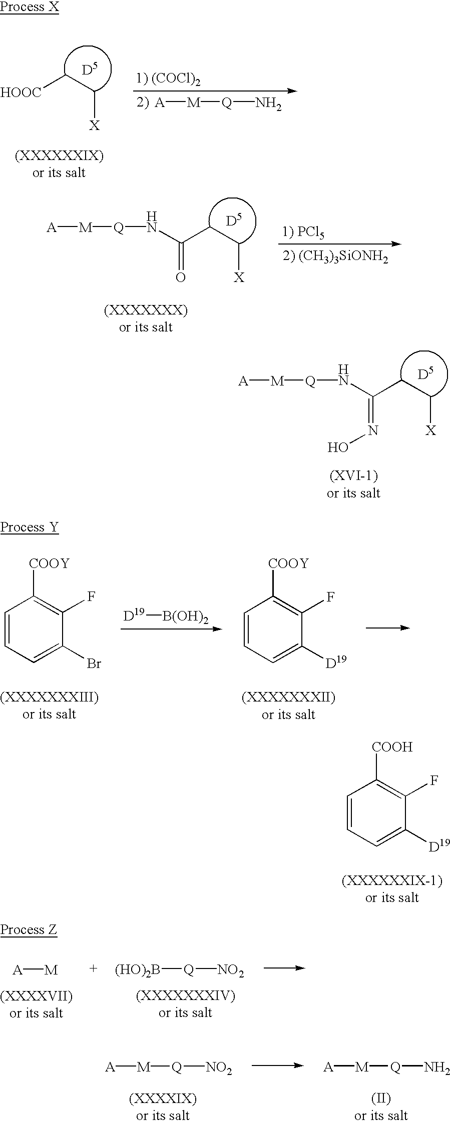

wherein A, M, (b), Q, R and X are each as defined above,

Z is hydrogen or Y as illustrated in the above, Y is a lower alkyl group and \mathring{p}—is a protective group for primary amino group known in the art.

The process for preparing the compounds (I) and their salts is explained in detail in the following.

Process 1

The object compound (I) and its salt can be prepared by reacting an amine compound (II) or its salt with a compound (III-1) or (III-2) or its salt.

Suitable salts of the compound (II) and the compound (III-1) or (III-2) can be referred to those as exemplified for the compound (I).

The reaction is usually carried out without solvent or in a conventional organic solvent which does not adversely influence the reaction such as toluene, dimethoxyethane, dimethylformamide, or a mixture thereof. The reaction is usually carried out under heating, for example, at a temperature of 100 to 250° C.

Process 2

The object compound (I-1) and its salt can be prepared by reacting a thioimidate ester compound (IV) or its salt with an amine compound (V) or its salt.

Suitable salts of the compound (IV) and the compound (V) can be referred to those as exemplified for the compound(I).

The reaction is usually carried out in a conventional solvent such as alcohol [e.g., methanol, ethanol, isopropyl alcohol], toluene, N,N-dimethylformamide or any other organic solvent which does not adversely affect the reaction, or a mixture thereof.

The reaction is preferably carried out under heating, for example, at a temperature of 60 to 150° C. However, the reaction temperature is not limited.

Process 3

The object compound (I-2) and its salt can be prepared by reacting a compound (VI) or its salt with an amine compound (VII) or its salt in a manner similar to the above Process 1.

Process 4

The object compound (I-3) and its salt can be prepared by reacting a compound (VIII) or its salt with methylamine in the presence of an organic acid (e.g., acetic acid).

Suitable salt of the compound (VIII) can be referred to those as exemplified for the compound (I).

The reaction is usually carried out in a conventional solvent such as water, acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out at a temperature under cooling to ambient temperature. However, the reaction temperature is not critical.

Process 5

The object compound (I-4) and its salt can be prepared by reacting a thiourea compound (IX) or its salt with a compound (X) or its salt.

Suitable salt or the compound (IX) and (X) can be referred to those as exemplified for the compound (I).

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out from at ambient temperature to under heating at reflux. However, the reaction temperature is not critical.

Process 6

The object compound (I-5) or (I-6) and its salt can be prepared by reacting a thiourea compound (IX) or its salt with a compound (XI-1) or (XI-2) or its salt.

Suitable salt or the compound (IX), (XI-1) and (XI-2) can be referred to those as exemplified for the compound (I).

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out under heating at a temperature of 40 to 150° C. However, the reaction temperature is not critical.

Process 7

The object compound (I-7) and its salt can be prepared by reacting a guanidine compound (XII) or its salt with a compound (XIII) or its salt in the presence of a base such as organic bases (e.g., trimethylamine, triethylamine, pyridine, picoline, dicyclohexylamine or dibenzylethylenediamine) or alkoxides (e.g., sodium methoxide or potassium methoxide).

Suitable salt or the compound (XII) and (XIII) can be referred to those as exemplified for the compound (I).

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out under heating at a temperature of 40 to 150° C. However, the reaction temperature is not critical.

Process 8

The object compound (I) and its salt can be prepared by treating a formyl compound (XIV) or its salt with a base such as an alkali metal hydroxide [e.g., sodium hydroxide or potassium hydroxide], an alkali metal hydrogen carbonate [e.g., sodium hydrogen carbonate or potassium hydrogen carbonate], an alkali metal carbonate [e.g., sodium carbonate], an alkali earth metal carbonate [e.g., calcium carbonate] and the like.

Suitable salt or the compound (XIV) can be referred to those as exemplified for the compound (I).

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out under heating at a temperature of 40 to 150° C. However, the reaction temperature is not critical.

Process 9

The object compound (I-8) and its salt can be prepared by treating a thiourea compound (XV) or its salt with a condensing agent.

Suitable salt or the compound (XV) can be referred to those as exemplified for the compound (I).

Suitable condensing agents include carbodiimide [e.g., N,N-dicyclohexylcarbodiimide, N-cyclohexyl-N′-(4-diethylaminocyclohexyl)carbodiimide, N-ethyl-N′-(3-dimethylaminopropyl)carbodiimide, or hydrochloride thereof], diphenylphosphinic azide, diphenylphosphinic chloride, diethylphosphoryl cyanide, bis(2-oxo-3-oxazolidinyl)phosphinic chloride, N,N′-carbonyldiimidazole, 2-ethoxy-1-ethoxycarbonyl-1,2-dihydroquinoline, cyanuric chloride and the like.

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out under heating at a temperature of 40 to 150° C. However, the reaction temperature is not critical.

Process 10

The object compound (I-9) or its salt can be prepared by treating a compound (XVI-1) or its salt with a base such as an alkali metal alkoxide [e.g., sodium methoxide, potassium ethoxide or potassium tert-butoxide], an alkali earth metal alkoxide [e.g., calcium ehthoxide or potassium methoxide] and the like. The object compound (I-10) or its salt can be prepared by treating a compound (XVI-2) or its salt with a base.

Suitable salt or the compound (XVI-1) or (XVI-2) can be referred to those as exemplified for the compound (I).

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide, N-methyl-2-pyrrolidone or any other organic solvent which does not adversely affect the reaction, or a mixture thereof.

The reaction is preferably carried out under heating at a temperature of 40 to 150° C. However, the reaction temperature is not critical.

Process 11

The object compound (I-11) or its salt can be prepared by reacting a compound (XVII) or its salt with a boronic acid compound D⁷—B(OH)₂ in the presence of palladium compound such as tetrakis(triphenylphospine)palladium(0) and a base such as an alkali metal carbonate [e.g., sodium carbonate], an alkali earth metal carbonate [e.g., calcium carbonate] and the like.

Suitable salt or the compound (XVII) can be referred to those as exemplified for the compound (I).

The reaction is usually carried out in a conventional solvent such as acetone, alcohol [e.g., methanol, ethanol or isopropyl alcohol], tetrahydrofuran, dioxane, 1,2-dimethoxyethane, toluene, methylene chloride, chloroform, N,N′-dimethylformamide or any other organic solvent which does not adversely affect the reaction, or a mixture thereof.

The reaction is preferably carried out under heating at a temperature of 40 to 150° C. However, the reaction temperature is not critical.

Process 12

The object compound (I-12) or its salt can be prepared by reacting a compound (XVIII) or its salt with an alcohol compound D⁹—OH in the presence of n alkali metal hydride [e.g., sodium hydride or potassium hydride].

Suitable salt or the compound (XVIII) can be referred to those as exemplified for the compound (I).

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out under heating at a temperature of 40 to 150° C. However, the reaction temperature is not critical.

Process 13

The object compound (I-13) or its salt can be prepared by reacting a compound (XVIII) or its salt with an alcohol compound D⁹—NH₂.

Suitable salt or the compound (XVIII) can be referred to those as exemplified for the compound (I).

The reaction is usually carried out in a basic conventional solvent such as pyridine or any other organic solvent which does not adversely affect the reaction, or a mixture thereof.

The reaction is preferably carried out under heating at a temperature of 40 to 150° C. However, the reaction temperature is not critical.

Process 14

The object compound (I) or its salt can be prepared by reacting a compound (XIX) or its salt with a compound (XX) in the presence of an alkali metal alkoxide [e.g., sodium methoxide, potassium ethoxide, sodium tert-butoxide or potassium tert-butoxide], phosphine compound such as biphenyl-2-yl-di-tert-butylphosphine, palladium compound such as tris(dibenzylideneacetone)dippaladium.

Suitable salt or the compounds (XIX) and (XX) can be referred to those as exemplified for the compound (I).

The reaction is usually carried out in a conventional solvent such as acetone, alcohol [e.g., methanol, ethanol or isopropyl alcohol], tetrahydrofuran, dioxane, toluene, methylene chloride, chloroform, N,N′-dimethylformamide or any other organic solvent which does not adversely affect the reaction, or a mixture thereof.

The reaction is preferably carried out under heating at a temperature of 40 to 150° C. However, the reaction temperature is not critical.

Process A

Compounds (V-1), (V-2) and (V-3) can be prepared from a compound (XXI) according to a method described in Reference Examples 24 to 31 or a similar method thereto.

Process B

Compounds (III-3), (V-4) and (VII-2) can be prepared from a compound (XVII) according to a method described in Reference Examples 1 to 23, 41, 94 to 97 or a similar method thereto.

Process C

A compound (VIII) can be prepared from compounds (XXXI) and (III) according to a method described in Reference Examples 33 to 36 or a similar method thereto.

Process D

A compound (III-5) can be prepared from a compound (III-4) according to a method described in Reference Example 32 or a similar method thereto.

Process E

A compound (III-6) and (III-7) can be prepared from a compound (XXXV) and (XXXVII), respectively, according to a method described in Reference Examples 37 to 40 or a similar method thereto.

Process F

A compound (VII) can be prepared from a compound (III) according to a method described in Reference Example 42 or a similar method thereto.

Process G

Compound (III-8) can be prepared from a compound (XXXIX) according to a method described in Reference Examples 58 and 59 or a similar method thereto.

Process H

Compound (XII) can be prepared from a compound (II) according to a method described in Reference Examples 60 and 61 or a similar method thereto.

Process I

Compound (III-9) can be prepared from a compound (XXXXI) according to a method described in Reference Examples 62, 63, 70 and 71 or a similar method thereto.

Process J

Compound (III-10) can be prepared from a compound (XXXXIII) according to a method described in Reference Examples 64 and 65 or a similar method thereto.

Process K

Compound (XIII) can be prepared from a compound (XXXXV) according to a method described in Reference Examples 66 and 67 or a similar method thereto.

Process L

Compound (XX-1) can be prepared from a compound (XXXXVI) according to a method described in Reference Example 68 or a similar method thereto.

Process M

Compound (XIX) can be prepared from compounds (XXXXVII) and (XXXXVIII) according to a method described in Reference Example 69 or a similar method thereto.

Process N

Compound (III) can be prepared from a compound (XXXXIX) according to a method described in Reference Example 102 or a similar method thereto.

Process O

Compound (III-11) can be prepared from a compound (XXXXX) according to a method described in Reference Examples 43 to 48 or a similar method thereto.

Process P

Compound (IX) can be prepared from a compound (II) according to a method described in Reference Examples 49 to 54 or a similar method thereto.

Process Q

Compound (V-5) can be prepared from a compound (XXXXXIV) according to a method described in Reference Examples 55 to 57 or a similar method thereto.

Process R

Compound (XIV-1) can be prepared from a compound (II) according to a method described in Reference Examples 72 to 74 or a similar method thereto.

Process S

Compound (V) can be prepared from a compound (XXXXXIX) according to a method described in Reference Examples 75 to 78 or a similar method thereto.

Process T

Compound (XV) can be prepared from a compound (II) according to a method described in Reference Examples 87 and 88 or a similar method thereto.

Process U

Compound (V-6) can be prepared from a compound (XXXXXX) according to a method described in Reference Examples 89 to 91 or a similar method thereto.

Process V

Compound (III-12) can be prepared from a compound (XXXXXXIV) according to a method described in Reference Examples 93 or a similar method thereto.

Process W

Compound (III-13) can be prepared from a compound (XXXXXXV) according to a method described in Reference Examples 92 and 98 to 101 or a similar method thereto.

Process X

Compound (XVI-1) can be prepared from a compound (XXXXXXIX) according to a method described in Reference Examples 79 to 82 or a similar method thereto.

Process Y

Compound (XXXXXXIX-1) can be prepared from a compound (XXXXXXXI) according to a method described in Reference Examples 83 to 86 or a similar method thereto.

Process Z

Compound (II) can be prepared from a compound (XXXXVII) and a compound (XXXXXXXIV) according to a method described in Reference Examples 106 to 108 or a similar method thereto.

The compound (I) of the present invention can be isolated and purified in a conventional manner, for example, extraction, precipitation, fractional crystallization, recrystallization, chromatography, or the like.

The compound (I) thus obtained can be converted to an optional salt by a conventional method.

The compounds (I) and salts thereof may include solvates [e.g., hydrate, methanolate, enclosure compound].

Among the starting compounds (II) to (VIII), novel compounds can be prepared by a method described in the following Examples or a similar method thereto.

The compounds (I) of the present invention may exhibit pharmacological activities such as 5-HT antagonism, especially, 5-HT_{2C} antogonism, and therefore are useful as 5-HT antagonist for treating or preventing central nervous system (CNS) disorders such as anxiety, depression, obsessive compulsive disorders, migraine, anorexia, Alzheimer's disease, sleep disorders, bulimia, panic attacks, withdrawal from drug abuse (e.g., with cocaine, ethanol, nicotine and benzodiazepines), schizophrenia, and also disorders associated with spinal trauma and/or head injury such as hydrocephalus, and the like.

Therefore, the compounds (I), its prodrug and salt thereof are useful for the treatment or prevention of the central nervous system (CNS) disorders such as anxiety, depression, obsessive compulsive disorders, migraine, anorexia, Alzheimer's disease, sleep disorders, bulimia, panic attacks, withdrawal from drug abuse (e.g., with cocaine, ethanol, nicotine and benzodiazepines), schizophrenia, and also disorders associated with spinal trauma and/or head injury such as hydrocephalus, and the like.

For therapeutic or preventive administration, the compound (I) of the present invention can be in a form of a pharmaceutical preparation, for example, in solid, semisolid or liquid form, which contains a compound (I), as an active ingredient, in admixture with a pharmaceutically acceptable carrier or excipient suitable for external, enteral, intravenous, intramuscular, parenteral or intramucous applications. The compound (I) may be compounded, for example, with the conventional non-toxic, pharmaceutically acceptable carriers for ointment, cream, plaster, tablets, pellets, capsules, suppositories, solution (saline, for example), emulsion, suspension (olive oil, for example), aerosols, pills, powders, syrups, injections, troches, cataplasms, aromatic waters, lotions, buccal tablets, sublingual tablets, nasal drops and any other form suitable for use. The carriers which can be used are water, wax, glucose, lactose, gum acacia, gelatin, mannitol, starch paster, magnesium trisilicate, talc, corn starch, keratin, paraffin, colloidal silica, potato starch, urea and other carriers suitable for use in manufacturing preparations, in solid, semisolid, or liquid form, and in addition auxiliary, stabilizing, thickening and coloring agents and perfumes may be used. The compound (I), its prodrug or a pharmaceutically acceptable salt thereof is included in a pharmaceutical composition in an effective amount sufficient for producing the desired effect upon the process or condition of the diseases, i.e. for the use of treatment and/or prevention of anxiety, depression, obsessive compulsive disorders, migraine, anorexia, Alzheimer's disease, sleep disorders, bulimia, panic attacks, withdrawal from drug abuse, schizophrenia, and also disorders associated with spinal trauma and/or head injury.

If needed, there may be included in the above preparations auxiliary substance, stabilizing agent, wetting agent and/or other commonly used additive such as lactose, citric acid, tartaric acid, stearic acid, magnesium stearate, terra alba, sucrose, corn starch, talc, gelatin, agar, pectin, peanut oil, olive oil, cacao butter, ethylene glycol, and the like.

The dosage of the compound (I) may depend on the age, conditions of the patient, kind of disease, kind of the compound (I) to be applied, etc., but in general, 0.01-500 mg of a compound (I) may be administered to an adult patient per day.

An average single dose of about 0.05 mg, 0.1 mg, 0.25 mg, 0.5 mg, 1 mg, 20 mg, 50 mg, 100 mg of the compound (I) may be used in treating the disease.

The following Examples are given for illustrating the present invention in more detail, but it is to be noted that the scope of the present invention is not limited by these Examples.

### BEST MODE FOR CARRYING OUT THE INVENTION

The following Examples are given only for the purpose of illustrating the present invention in more detail.

### REFERENCE EXAMPLE 1

To a solution of 5-bromoisoquinoline (1.5 g) in a mixture of dimethoxyethane (25 ml) and an aqueous sodium carbonate solution (2 M, 12 ml) were added phenylboronic acid (1.31 g) and tetrakis(triphenylphosphine)palladium (0) (0.17 g) under nitrogen. The mixture was heated to 100° C. for 3 hours. After cooling to ambient temperature, the separated organic layer was evaporated under reduced pressure. The residue was taken up into ethyl acetate. The resulting solution was washed in turn with an aqueous potassium carbonate solution (10%) and brine, dried over sodium sulfate and evaporated to dryness. The residue was purified by a column chromatography on silica gel (100 ml) eluting with 0-20% ethyl acetate in n-hexane to give 5-phenylisoquinoline (1.50 g).

APCI-mass; 206 (m/z, [M+H]⁺); NMR (DMSO-d₆, δ): 7.40-7.80 (8H, m), 8.18 (1H, dd, J=1.9, 7.0 Hz), 8.49 (1H, d, J=6.0 Hz), 9.40 (1H, s).

### REFERENCE EXAMPLE 2

To a solution of 5-phenylisoquinoline (0.39 g) in dichloromethane (5 ml) was added m-chloroperbenzoic acid (0.42 g) at ambient temperature. After stirring for 6 hours at ambient temperature, the reaction mixture was taken up into ethyl acetate. The resulting mixture was washed in turn with an aqueous potassium carbonate solution (10%) and brine, dried over potassium carbonate and evaporated to dryness under reduced pressure. The residue was purified by a column chromatography on silica gel (50 ml) eluting with 0-5% ethyl acetate in n-hexane to give 5-phenylisoquinoline-2-oxide, which was used for the next step without purification.

### REFERENCE EXAMPLE 3

To phosphorous oxychloride (5 ml) was added 5-phenylisoquinoline-2-oxide (crude) by portions, and the mixture was heated to 100° C. for 30 minutes. The mixture was evaporated under reduced pressure to dryness. The residue was taken up into a mixture of ethyl acetate and water, and the pH of the mixture was adjusted to around 7.5 with an aqueous sodium hydroxide solution. The separated organic layer was washed with brine, dried over sodium sulfate and evaporated to dryness. The residue was purified by a column chromatography on silica gel (50 ml) eluting with 0-15% ethyl acetate in n-hexane to give 1-chloro-5-phenylisoquinoline (166 mg).

APCI-mass; 240 (m/z, [M+H]⁺), NMR (DMSO-d₆, δ): 7.48-7.70 (6H, m), 7.81-7.97 (2H, m), 8.30 (1H, d, J=5.9 Hz), 8.36 (1H, d, J=7.2 Hz).

### REFERENCE EXAMPLE 4

A mixture of 1-chloro-5-phenylisoquinoline (0.577 g) and 3-nitroaniline (0.997 g) was heated to 190° C. for 5 minutes. After cooling to ambient temperature, the reaction mixture was taken up into a mixture of ethyl acetate and an aqueous potassium carbonate solution (10%). The separated organic layer was washed with brine, dried over potassium carbonate and evaporated to dryness. The crystalline residue was triturated with diisopropyl ether to give (3-nitrophenyl)-(5-phenylisoquinolin-1-yl)amine (0.74 g).

APCI-mass; 342 (m/z, [M+H]⁺), NMR (DMSO-d₆, δ): 7.13 (1H, d, J=6.1 Hz), 7.47-7.83 (9H, m), 8.07 (1H, d, J=6.0 Hz), 8.32-8.42 (1H, m), 8.55-8.67 (1H, m), 8.96 (1H, dd, J=2.2, 2.2 Hz), 9.73 (1H, s).

### REFERENCE EXAMPLE 5

To a solution of (3-nitrophenyl)-(5-phenylisoquinolin-1-yl)amine (0.72 g) in a mixture of methanol (5 ml) and tetrahydrofuran (15 ml) was added palladium on carbon (10%, 50% wet, 0.14 g) under nitrogen. The resultant mixture was allowed to stir under atmospheric pressure of hydrogen for 5 hours. The catalyst was removed by filtration and the filtrate was evaporated under reduced pressure to give (3-aminophenyl)-(5-phenylisoquinolin-1-yl)amine.

APCI-mass; 312 (m/z, [M+H]⁺), NMR (DMSO-d₆, δ): 4.98 (2H, brs), 6.20-6.35 (1H, m), 6.95 (3H, d, J=5.4 Hz), 7.17 (1H, s), 7.40-7.72 (7H, m), 7.93 (1H, d, J=6.0 Hz), 8.51-8.60 (1H, m), 8.95 (1H, s).

### REFERENCE EXAMPLE 6

A mixture of 1-chloro-5-phenylisoquinoline (0.15 g) and 3-(3-aminobenzyl)carbamic acid benzyl ester (320 mg) was heated to 190° C. for 15 minutes. After cooling to ambient temperature, the reaction mixture was taken up into a mixture of ethyl acetate and an aqueous potassium carbonate solution (10%). The separated organic layer was washed with brine, dried over potassium carbonate and evaporated under reduced pressure to dryness. The residue was triturated with a mixture of methanol and diisopropyl ether to give [3-(5-phenylisoquinolin-1-ylamino)benzyl]carbamic acid benzyl ester (246 mg).

APCI-mass; 460 (m/z, [M+H]⁺), NMR (CDCl₃, δ): 4.43 (2H, d, J=6.0 Hz), 5.15 (2H, s), 6.98 (1H, d, J=7.5 Hz), 7.05-7.70 (17H, m), 7.80-7.94 (1H, m), 7.94-8.10 (2H, m),.

### REFERENCE EXAMPLE 7

To a solution of [3-(5-phenylisoquinolin-1-ylamino)benzyl]-carbamic acid benzyl ester (216 mg) in tetrahydrofuran (5 ml) was added palladium on carbon (10%, 50% wet, 35 mg) under nitrogen. The resultant mixture was allowed to stir under atmospheric pressure of hydrogen for 5 hours. The catalyst was removed by filtration and the filtrate was evaporated under reduced pressure to give (3-aminomethylphenyl)-(5-phenylisoquinolin-1-yl)amine (127 mg).

APCI-mass ; 326 (m/z, [M+H]⁺), NMR (DMSO-d₆, δ): 1.85 (2H, brs), 3.75 (2H, s), 6.99 (1H, d, J=7.6 Hz), 7.19-7.35 (1H, m), 7.38-7.59 (5H, m), 7.59-7.86 (5H, m), 7.92 (1H, s), 8.62 (1H, d, J=8.8 Hz), 9.23 (1H, s).

### REFERENCE EXAMPLE 8

To a solution of 5-bromoisoquinoline (1.5 g) in a mixture of dimethoxyethane (25 ml) and an aqueous sodium carbonate solution (2 M, 12 ml) were added 3-thiopheneboronic acid (1.38 g) and tetrakis(triphenylphosphine)palladium (0) (0.17 g) under nitrogen, and the mixture was heated to 100° C. for 3 hours. After cooling to ambient temperature, the separated organic layer was evaporated under reduced pressure. The residue was taken up into ethyl acetate. The mixture was washed in turn with an aqueous potassium carbonate solution (10%) and brine, dried over sodium sulfate and evaporated to dryness. The residue was purified by a column chromatography on silica gel (100 ml) eluting with 0-20% ethyl acetate in n-hexane to give 5-(thiophen-3-yl)isoquinoline (1.43 g).

APCI-mass; 212 (m/z, [M+H]⁺), NMR (DMSO-d₆, δ): 7.35-7.50 (1H, m), 7.70-7.90 (5H, m), 8.14 (1H, d, J=7.6 Hz), 8.52 (1H, d, J=6.0 Hz), 9.38 (1H, s).

### REFERENCE EXAMPLE 9

To a solution of 5-(thiophen-3-yl)isoquinoline (1.41 g) in dichloromethane (20 ml) was added m-chloroperbenzoic acid (2.14 g) at ambient temperature. After stirring at ambient temperature for 6 hours, the reaction mixture was taken up into ethyl acetate. The mixture was washed in turn with an aqueous sodium hydroxide solution (4N) and brine, dried over potassium carbonate and evaporated under reduced pressure to dryness. The residue was triturated with diisopropyl ether to give 5-(thiophen-3-yl)isoquinoline 2-oxide (1.25 g).

APCI-mass; 228 (m/z, [M+H]⁺), NMR (DMSO-d₆, δ): 7.35-7.45 (1H, m), 7.55-7.97 (6H, m), 8.14 (1H, dd, J=1.9, 7.3 Hz), 9.01 (1H, d, 1.8 Hz).

### REFERENCE EXAMPLE 10

To phosphorous oxychloride (6 ml) was added 5-(thiophen-3-yl)isoquinoline 2-oxide (1.2 g) by portions, and the mixture was heated to 100° C. for 30 minutes. The mixture was evaporated under reduced pressure to dryness. The residue was taken up into a mixture of ethyl acetate and water. The pH of the mixture was adjusted to around 7.5 with an aqueous sodium hydroxide solution. The separated organic layer was washed with brine, dried over sodium sulfate and evaporated to dryness. The residue was triturated with a mixture of ethyl acetate and diisopropyl ether to give 1-chloro-5-(thiophen-3-yl)isoquinoline (0.53 g).

APCI-mass; 246 (m/z, [M+H]⁺), NMR (DMSO-d₆, δ): 7.39 (1H, dd, J=2.2, 4.2 Hz), 7.80-8.00 (5H, m), 8.23-8.40 (2H, m).

### REFERENCE EXAMPLE 11

To a solution of 5-bromoisoquinoline (0.266 g) in dichloromethane (5 ml) was added m-chloroperbenzoic acid (0.27 g) at ambient temperature. After stirring at ambient temperature for 6 hours, the reaction mixture was taken up into ethyl acetate. The mixture was washed in turn with an aqueous potassium carbonate solution (10%) and brine, dried over potassium carbonate and evaporated under reduced pressure. The residue was triturated with diisopropyl ether to give 5-bromoisoquinoline-2-oxide (0.28 g).

APCI-mass; 224, 226 (m/z, [M+H]⁺), NMR (DMSO-d₆, δ): 7.58 (1H, t, J=7.7 Hz), 7.88-8.08 (3H, m), 8.27 (1H, dd, J=1.8, 7.4 Hz), 9.03 (1H, d, J=1.8 Hz).

### REFERENCE EXAMPLE 12

To phosphorous oxychloride (1.4 ml) was added 5-bromoisoquinoline-2-oxide (0.28 g), and the mixture was heated to 100° C. for an hour. The mixture was evaporated under reduced pressure. The residue was taken up into a mixture of ethyl acetate and water and the pH of the mixture was adjusted to around 7.5 with an aqueous sodium hydroxide solution. The separated organic layer was washed with brine, dried over sodium sulfate and evaporated. The residue was triturated with diisopropyl ether to give 1-chloro-5-bromoisoquinoline (0.281 g).

APCI-mass; 242, 244 (m/z, [M+H]⁺), NMR (DMSO-d₆, δ): 7.76 (1H, t, J=7.7 Hz), 8.02 (1H, d, J=5.8 Hz), 8.29 (1H, d, J=7.7 Hz), 8.36 (1H, d, J=7.7 Hz), 8.47 (1H, d, J=5.8 Hz).

### REFERENCE EXAMPLE 13

A mixture of 1-chloro-5-bromoisoquinoline (0.7 g) and 3-nitroaniline (0.997 g) was heated to 190° C. for 3 minutes. After cooling to ambient temperature, the reaction mixture was taken up into a mixture of dichloromethane and an aqueous potassium carbonate solution (10%). The separated organic layer was washed with brine, dried over potassium carbonate and evaporated. The crystalline residue was triturated with diisopropyl ether to give (5-bromoisoquinolin-1-yl)-(3-nitorophenyl)amine (1.27 g).

APCI-mass; 344, 346 (m/z, [M+H]⁺), NMR (DMSO-d₆, δ): 7.45 (1H, d, J=6.0 Hz), 7.53-7.72 (2H, m), 7.80-7.92 (1H, m), 8.13 (1H, d, J=7.1 Hz), 8.23 (1H, d, J=6.0 Hz), 8.35 (1H, d, J=8.2 Hz), 8.62 (1H, d, J=8.5 Hz), 8.91 (1H, t, J=2.1 Hz), 9.78 (1H, s).

### REFERENCE EXAMPLE 14

To a solution of (5-bromoisoquinolin-1-yl)-(3-nitrophenyl)amine (0.3 g) in a mixture of ethanol (6 ml) and water (6 ml) were added ammonium chloride (20 mg), iron powder (170 mg) and 2 drops of 6N hydrochloric acid. The resultant mixture was heated to 110° C. for 5 hours. After cooling to ambient temperature, the precipitate was removed by filtration with Celite. The filtrate was diluted with dichloromethane and washed in turn with an aqueous potassium carbonate solution and brine. The solution was dried over potassium carbonate and evaporated under reduced pressure. The residue was triturated with diisopropyl ether to give (3-aminophenyl)-(5-bromoisoquinolin-1-yl)amine (172 mg).

APCI-mass; 314, 316 (m/z, [M+H]⁺), NMR (DMSO-d₆, δ): 4.99 (2H, brs), 6.27 (1H, d, J=6.9 Hz), 6.85-7.03 (2H, m), 7.00 (1H, s), 7.26 (1H, d, J=6.3 Hz), 7.50 (1H, t, J=8.1 Hz), 7.99-8.13 (2H, m), 8.55 (1H, d, J=8.3 Hz), 9.03 (1H, s).

### REFERENCE EXAMPLE 15

A mixture of 1-chloroisoquinoline (0.577 g) and 3-nitroaniline (0.997 g) was heated to 190° C. for 3 minutes. After cooling to ambient temperature, the reaction mixture was taken up into a mixture of ethyl acetate and an aqueous potassium carbonate solution (10%). The separated organic layer was washed with brine, dried over potassium carbonate and evaporated to dryness. The crystalline residue was triturated with diisopropyl ether to give (3-nitorophenyl)-(isoquinolin-1-yl)amine (0.55 g).

APCI-mass; 266 (m/z, [M+H]⁺), NMR (DMSO-d₆, δ): 7.31 (1H, d, J=5.8 Hz), 7.50-7.93 (5H, m), 8.10 (1H, d, J=5.7 Hz), 8.30-8.40 (1H, m), 8.57 (1H, d, J=8.4 Hz), 8.97 (1H, t, J=2.1 Hz), 9.65 (1H, s).

### REFERENCE EXAMPLE 16

To a solution of (3-nitrophenyl)-(isoquinolin-1-yl)amine (20 g) in a mixture of methanol (10 ml) and tetrahydrofuran (10 ml) was added palladium on carbon (10%, 50%, wet, 0.3 g) under nitrogen. The resultant mixture was allowed to stir under atmospheric pressure of hydrogen for 3 hours. The catalyst was removed by filtration and the filtrate was evaporated under reduced pressure. The residue was triturated with a mixture of ethyl acetate and diisopropyl ether to give (3-aminophenyl)-(isoquinolin-1-yl)amine.

APCI-mass; 236 (m/z, [M+H]⁺) NMR (DMSO-d₆, δ): 4.99 (2H, brs), 6.20-6.22 (1H, m), 6.95 (2H, d, J=5.2 Hz), 7.10-7.20 (2H, m), 7.50-7.82 (3H, m), 7.95 (1H, d, J=5.7 Hz), 8.50 (1H, d, J=8.3 Hz), 8.87 (1H, s)

### REFERENCE EXAMPLE 17

To a solution of 4-bromoisoquinoline (1.5 g) in a mixture of dimethoxyethane (25 ml) and an aqueous sodium carbonate solution (2 M, 11.9 ml) were added phenylboronic acid (1.31 g) and tetrakis(triphenylphosphine)palladium (0) (0.17 g) under nitrogen. The mixture was heated to 100° C. for 3 hours. After cooling to ambient temperature, the separated organic layer was evaporated under reduced pressure. The residue was taken up into ethyl acetate. The mixture was washed in turn with an aqueous potassium carbonate solution (10%) and brine, dried over sodium sulfate and evaporated to dryness., The residue was purified by a column chromatography on silica gel (100 ml) eluting with 0-15% ethyl acetate in n-hexane to give 4-phenylisoquinoline (1.22 g),

APCI-mass; 206 (m/z, [M+H]⁺) NMR (DMSO-d₆, δ): 7.50-7.65 (5H, m), 7.70-7.88 (3H, m), 7.87-8.26 (1H, m), 8.45 (1H, s), 9.36 (1H, s).

### REFERENCE EXAMPLE 18

To a solution of 4-phenylisoquinoline (1.21 g) in dichloromethane (10 ml) were added m-chloroperbenzoic acid (1.3 g) at ambient temperature. After stirring at ambient temperature for 6 hours, the reaction mixture was taken up into ethyl acetate. The mixture was washed in turn with an aqueous potassium carbonate solution (10%) and brine, dried over potassium carbonate and evaporated under reduced pressure to dryness. The residue was triturated with diisopropyl ether to give 4-phenylisoquinoline-2-oxide (1.21 g).

APCI-mass; 222 (m/z, [M+H]⁺) NMR (DMSO-d₆, δ): 7.50-7.75 (8H, m), 7.95-8.01 (1H, m), 8.09 (1H, d, J=1.8 Hz), 9.01 (1H, d, J=1.8 Hz)

### REFERENCE EXAMPLE 19

To phosphorous oxychloride (5 ml) was added 4-phenylisoquinoline-2-oxide (1.10 g) by portions, and the mixture was heated to 100° C. for an hour. The mixture was evaporated under reduced pressure to dryness. The residue was taken up into a mixture of ethyl acetate and water, and the pH of the mixture was adjusted to around 7.5 with an aqueous sodium hydroxide solution. The separated organic layer was washed with brine, dried over sodium sulfate and evaporated. The residue was triturated with diisopropyl ether to give 1-chloro-4-phenylisoquinoline (1.03 g).

APCI-mass; 240 (m/z, [M+H]⁺) NMR (DMSO-d₆, δ): 7.50-7.60 (5H, m), 7.86-7.91 (3H, m), 8.26 (1H, s), 8.37-8.43 (1H, m)

### REFERENCE EXAMPLE 20

To a solution of 5-bromoisoquinoline (1.5 g) in a mixture of dimethoxyethane (25 ml) and an aqueous sodium carbonate solution (2M, 12 ml) were added 4-fluorophenylboronic acid (1.51 g) and tetrakis (triphenylphosphine) palladium (0) (0.17 g) under nitrogen, and the mixture was heated to 100° C. for 3 hours. After cooling to ambient temperature, the separated organic layer was evaporated under reduced pressure. The residue was taken up into ethyl acetate. The mixture was washed in turn with an aqueous potassium carbonate solution (10%) and brine, dried over sodium sulfate and evaporated. The residue was purified by a column chromatography on silica gel (100 ml) eluting with 0-20% ethyl acetate in n-hexane to give 5-(4-fluorophenyl)-isoquinoline (1.59 g).

APCI-mass; 224 (m/z, [M+H]⁺) NMR (DMSO-d₆, δ): 7.34-7.45 (2H, m), 7.50-7.65 (3H, m), 7.70-7.82 (2H, m), 8.18 (1H, dd, J=2.5, 6.8 Hz), 8.50 (1H, d, J=6.0 Hz), 9.40 (1H, s).

### REFERENCE EXAMPLE 21

To a solution of 5-(4-fluorophenyl)isoquinoline (1.58 g) in dichloromethane (40 ml) was added m-chloroperbenzoic acid (2.44 g) at ambient temperature. After stirring at ambient temperature for 6 hours, the reaction mixture was dried over potassium carbonate twice and evaporated under reduced pressure. The residue was triturated with diisopropyl ether to give 5-(4-fluorophenyl)isoquinoline-2-oxide (1.42 g).

APCI-mass; 240 (m/z, [M+H]⁺) NMR (DMSO-d₆, δ): 7.40 (2H, t, J=8.9 Hz), 7.50-7.80 (5H, m), 7.92 (1H, d, J=8.2 Hz), 8.12 (1H, dd, J=1.9, 7.4 Hz), 9.04 (1H, d, J=1.9 Hz)

### REFERENCE EXAMPLE 22

To phosphorous oxychloride (7 ml) was added 5-(4-fluorophenyl)isoquinoline-2-oxide (1.40 g) by portions, and the mixture was heated to 100° C. for an hour. The mixture was evaporated under reduced pressure to dryness. The residue was taken up into a mixture of ethyl acetate and water, and the pH of the mixture was adjusted to around 7.5 with an aqueous sodium hydroxide solution. The separated organic layer was washed with brine, dried over sodium sulfate and evaporated. The residue was triturated with diisopropyl ether to give 1-chloro-5-(4-fluorophenyl)isoquioline (0.86 g).

APCI-mass; 258 (m/z, [M+H]⁺) NMR (DMSO-d₆, δ): 7.41 (2H, t, J=8.9 Hz), 7.50-7.67 (3H, m), 7.82-7.95 (2H, m), 7.29 (1H, d, J=5.9 Hz), 8.36 (1H, d, J=8.0 Hz)

### REFERENCE EXAMPLE 23

To a solution of 1,3-phenylenediamine (1.2 g) in tetrahydrofuran (10 ml) was added dropwise a solution of n-butyl lithium in n-hexane (1.54 M, 5.8 ml) at 0° C. The mixture was stirred at 0° C. for 30 minutes, and added to a solution of 3-chlorobenzo[d]isoxazole (0.30 g) in tetrahydrofuran (2 ml) at 0° C. The reaction mixture was allowed to stir at 0° C. for one hour, and was taken up into a mixture of ethyl acetate and water. The separated organic layer was washed well with water, dried over potassium carbonate. The organic layer was evaporated under reduced pressure to dryness. The residue was purified by a column chromatography on silica gel (60 ml) eluting with 0-1% methanol in dichloromethane. The obtained product was triturated with diisopropyl ether to give N-(benzo[d]isoxazol-3-yl)benzene-1,3-diamine (161 mg).

APCI-mass; 226 (m/z, [M+H]⁺) NMR (DMSO-d₆, δ): 5.12 (2H, brs), 6.19 (1H, d, J=7.7 Hz), 6.81 (1H, d, J=8.7 Hz), 6.95 (1H, d, J=7.9 Hz), 7.01 (1H, d, J=1.7 Hz), 7.30-7.40 (1H, m), 7.53-7.68 (2H, m), 8.15 (1H, d, J=7.8 Hz), 9.21 (1H, s).

### REFERENCE EXAMPLE 24

To a solution of 3-bromo-2-fluorobenzoic acid (2.3 g) in dichloromethane (20 ml) were added in turn oxalyl chloride (1.83 ml) and a catalytic amount of N,N-dimethylformamide (2 drops) at ambient temperature. After stirring at ambient temperature for an hour, the reaction mixture was evaporated in vacuo to give 3-bromo-2-fluorobenzoyl chloride. To a solution of 3-nitroaniline (1.45 g) in dichloromethane (20 ml) were added pyridine (2.54 ml) and the 3-bromo-2-fluorobenzoyl chloride solution in dichloromethane (5 ml) at 0° C. After stirring at ambient temperature for 2 hours, the reaction mixture was evaporated to dryness. The residue was taken up into water (100 ml). The resultant precipitate was collected by filtration and washed in turn with water and diisopropyl ether to give 3-bromo-2-fluoro-N-(3-nitrophenyl)benzamide (3.39 g).

APCI-mass; 339, 341 (m/z, [M+H]⁺) NMR (DMSO-d₆, δ): 7.32 (1H, t, J=4.6 Hz), 7.62-7.79 (2H, m), 7.89-8.10 (3H, m), 8.73 (1H, t, J=2.1 Hz), 11.04 (1H, s).

### REFERENCE EXAMPLE 25

A mixture of 3-bromo-2-fluoro-N-(3-nitrophenyl)benzamide (3.28 g) and phosphorous pentoxide (2.61 g) was heated to 65° C. for 6 hours. The mixture was evaporated under reduced pressure to dryness. The residue was triturated with diisopropyl ether to give 3-bromo-2-fluoro-N-(3-nitrophenyl)benzimidoyl chloride, which was used for further reaction without any purification.

### REFERENCE EXAMPLE 26

To a solution of 3-bromo-2-fluoro-N-(3-nitrophenyl)benzimidoyl chloride (crude) in tetrahydrofuran (60 ml) was added O-trimethylsilylhydroxylamine (5.0 g) at ambient temperature. After stirring at ambient temperature for 3 days, the mixture was added with hydrochloric aid (1N, 10 ml). The resultant mixture was taken up into a mixture of ethyl acetate and water, and the pH of the mixture was adjusted to around 7.5 with an aqueous sodium bicarbonate solution. The separated organic layer was washed with brine, dried over magnesium sulfate and evaporated under reduced pressure. The residue was purified by a column chromatography on silica gel (100 ml) eluting with 0-25% ethyl acetate in n-hexane. The obtained product was triturated with a mixture of toluene and diisopropyl ether to give 3-bromo-2-fluoro-N-hydroxy-N′-(3-nitrophenyl)benzamidine (2.82 g).

APCI-mass; 354, 356 (m/z, [M+H]⁺)

### REFERENCE EXAMPLE 27

To a solution of 3-bromo-2-fluoro-N-hydroxy-N′-(3-nitrophenyl)benzamidine (0.19 g) in N-methylpyrrolidone (8 ml) was added potassium tert-butoxide (68 mg) under nitrogen atmosphere, and the mixture was heated to 100° C. for 2 hours. After cooling to ambient temperature, the reaction mixture was taken up into a mixture of ethyl acetate and water. The separated organic layer was washed with brine, dried over potassium carbonate and evaporated under reduced pressure. The obtained product was triturated with diisopropyl ether to give (7-bromo-benzo[d]isoxazol-3-yl)-(3-nitorophenyl)amine as an 1:1 adduct with N-methylpyrrolidone (49 mg).

APCI-mass; 100, 334, 336 (m/z, [M+H]⁺) NMR (DMSO-d₆, δ): 1.80-2.00 (2H, m), 2.10-2.24 (2H, m), 2.70 (3H, s), 3.25-3.35 (2H, m), 7.38 (1H, t, J=8.0 Hz), 7.69 (1H, t, J=8.0 Hz), 7.80-8.00 (2H, m) 8.05 (1H, dd, J=1.4, 8.0 Hz), 8.16 (1H, d, J=7.2 Hz), 8.65 (1H, t, J=2.2 Hz), 10.26 (1H, s).

### REFERENCE EXAMPLE 28

To a solution of (7-bromo-benzo[d]isoxazol-3-yl)-(3-nitrophenyl)amine (1:1 adduct with N-methylpyrrolidone, 0.3 g) in a mixture of dimethoxyethane (3 ml) and an aqueous sodium carbonate solution (2 M, 1.5 ml) were added phenylboronic acid (0.17 g) and tetrakis(triphenylphosphine)palladium (0) (21 mg) under nitrogen. The mixture was heated to 100° C. for 2 hours. After cooling to ambient temperature, the separated organic layer was evaporated under reduced pressure. The residue was taken up into ethyl acetate. The mixture was washed in turn with an aqueous potassium carbonate solution (10%) and brine, dried over sodium sulfate and evaporated. The residue was triturated with methanol to give (3-nitrophenyl)-(7-phenylbenzo[d]isoxazol-3-yl)amine (170 mg).

APCI-mass; 332 (m/z, [M+H]⁺) NMR (DMSO-d₆, δ): 7.42-7.74 (5H, m), 7.89-7.99 (4H, m), 8.05-8.20 (2H, m), 8.73 (1H, t, J=2.2 Hz), 10.23 (1H, s).

### REFERENCE EXAMPLE 29

To a solution of (3-nitrophenyl)-(7-phenylbenzo[d]isoxazol-3-yl)amine(150 mg) in a mixture of methanol (7ml) and tetrahydrofuran(7 ml) was added palladium on carbon(10%, 50% wet, 35 mg) under nitrogen. The resultant mixture was allowed to stir under atmospheric pressure of hydrogen for an hour. The catalyst was removed by filtration and the filtrate was evaporated under reduced pressure. The residue was purified by a column chromatography on silica gel (50 ml) eluting with 0-30% ethyl acetate in n-hexane to give N-(7-phenylbenzo[d]isoxazol-3-yl)benzene-1,3-diamine (77 mg).

APCI-mass; 302 (m/z, [M+H]⁺) NMR (DMSO-d₆, δ): 5.11 (2H, brs), 6.21 (1H, d, J=7.8 Hz), 6.80-6.90 (1H, m), 7.00-7.05 (1H, m), 7.35-8.00 (9H, m), 9.26 (1H, s).

### REFERENCE EXAMPLE 30

To a solution of (7-bromo-benzo[d]isoxazol-3-yl)-(3-nitrophenyl)amine (0.3 g) in a mixture of ethanol (6 ml) and water (6 ml) were added ammonium chloride (15 mg) and iron powder (135 mg). The resultant mixture was heated to 110° C. for 45 minutes. After cooling to ambient temperature, the precipitate was removed by filtration with Cellite, and the filtrate was diluted with dichloromethane. The solution was washed in turn with an aqueous potassium carbonate solution and brine, dried over magnesium sulfate and evaporated under reduced pressure. The residue was purified by a column chromatography on silica gel (50 ml) eluting with 0-20% methanol in dichloromethane to give N-(7-bromobenzo[d]isoxazol-3-yl)benzene-1,3-diamine (118 mg).

APCI-mass; 304, 306 (m/z, [M+H]⁺) NMR (DMSO-d₆, δ): 5.14 (2H, brs), 6.22 (1H, d, J=7.9 Hz), 6.78-6.86 (1H, m), 6.90-7.10 (2H, m), 7.31 (1H, t, J=7.8 Hz), 7.86 (1H, d, J=7.5 Hz), 8.17 (1H, d, J=7.9 Hz), 9.32 (1H, s)

### REFERENCE EXAMPLE 31

To a solution of (7-bromobenzo[d]isoxazol-3-yl)-(3-nitro-phenyl)amine (0.3 g) in a mixture of methanol (5 ml) and tetrahydrofuran (5 ml) was added palladium on carbon (10%, 50% wet, 60 mg) under nitrogen. The resultant mixture was allowed to stir under atmospheric pressure of hydrogen for 45 minutes. The catalyst was removed by filtration and the filtrate was evaporated under reduced pressure. The residue was purified by a column chromatography on silica gel (50 ml) eluting with 2-40% ethyl acetate in n-hexane to, give N-(benzo[d]isoxazol-3-yl)benzene-1,3-diamine (42 mg).

APCI-mass; 304, 306 (m/z, [M+H]⁺).

### REFERENCE EXAMPLE 32

To a solution of 5-amino-1-chloroisoquinoline (1.0 g) in acetic acid (5 ml) was added 2,5-dimethoxytetrahydrofuran (0.73 ml), and the resultant mixture was heated to 100° C. for an hour. The mixture was evaporated to dryness. The residue was taken up into a mixture of dichloromethane and an aqueous potassium carbonate solution (10%). The separated organic layer was evaporated under reduced pressure. The residue was purified by a column chromatography on silica gel (50 ml) eluting with 0-2% methanol in dichloromethane. The obtained product was triturated with methanol to give 1-chloro-5-(pyrrol-1-yl)isoquinoline (0.55 g).

APCI-mass; 229 (m/z, [M+H]⁺), NMR (DMSO-d₆, δ): 6.38 (2H, t, J=2.1 Hz), 7.17 (2H, t, J=2.1 Hz), 7.54 (1H, dd, J=0.8, 5.9 Hz), 7.91 (2H, dd, J=0.8, 4.8 Hz), 8.2-8.4 (2H, m).

### REFERENCE EXAMPLE 33

1-[3-(Quinolin-2-ylamino)-phenyl]-ethanone as a yellow powder was prepared in a manner similar to Example 35.

m.p.: 181-183° C. IR (KBr, cm⁻¹): 3363, 1674 Mass: 263 (m/z, (M+H)⁺) NMR (DMSO-d₆, δ): 2.63 (3H, s), 7.08 (1H, d, J=8.9 Hz), 7.32 (1H, ddd, J=7, 7, 1.3 Hz), 7.48 (1H, dd, J=7.7, 7.7 Hz), 7.50-7.77 (4H, m), 8.10 (1H, d, J=8.9 Hz), 8.28 (1H, br d, J=7.9 Hz), 8.67 (1H, br s), 9.67 (1H, s).

### REFERENCE EXAMPLE 34

To a solution of 1-[3-(quinolin-2-ylamino)-phenyl]ethanone (1.31 g), pyridinium tribromide (1.60 g) and acetic acid (10 ml) at room temperature was added 2 ml of 30% hydrobromic acid in acetic acid. After stirring for an hour, the reaction mixture was poured into water (200 ml) and the insoluble materials were collected by filtration. The obtained material was dissolved in ethyl acetate. The solution was washed with brine, dried over magnesium sulfate, filtered and evaporated. The residue was crystallized from dichloromethane to give 2-bromo-1-[3-(quinolin-2-ylamino)-phenyl]ethanone (0.47 g) as a light yellow powder.

m.p.: 151-152° C. IR (KBr, cm⁻¹): 3381, 1682 Mass: 341, 343 (m/z, [M+H]⁺, bromide isomers) NMR (CDCl₃, δ): 4.51 (2H, s), 6.93 (1H, d, J=8.9 Hz), 7.36 (1H, ddd, J=8.1, 8.1, 1.1 Hz), 7.48 (1H, dd, J=7.9, 7.9 Hz), 7.60-7.70 (3H, m), 7.83-7.91 (2H, m), 7.99 (1H, d, J=8.9 Hz), 8.48 (1H, dd, J=2.0, 2.0 Hz).

### REFERENCE EXAMPLE 35

To a solution of 2-bromo-1-[3-(quinolin-2-ylamino)-phenyl]-ethanone (469 mg) in N,N-dimethylformamide (7 ml) at room temperature was added diformylimide sodium salt (196 mg). After an hour at room temperature, the reaction mixture was diluted with ethyl acetate and washed in turn with water and brine. The organic phase was dried with magnesium sulfate, filtered and evaporated.

The residue was purified by a column chromatography (silica gel, dichloromethane/methanol) to give N,N-diformyl-2-amino-1-[3-(quinolin-2-ylamino)-phenyl]-ethanone (0.42 g).

Mass: 334 (m/z, (M+H)⁺) NMR (CDCl₃, δ): 5.14 (2H, s), 6.92 (1H, d, J=8.9 Hz), 7.26-8.01 (9H, m), 8.46 (1H, s), 9.06 (2H, s).

### REFERENCE EXAMPLE 36

A solution of N,N-diformyl-2-amino-1-[3-(quinolin-2-ylamino)-phenyl]-ethanone (456 mg), dichloromethane (20 ml), methanol (20 ml), and triethylamine (1 ml) was stirred at room temperature for 3 hours. The reaction mixture was evaporated. The residue was purified by a column chromatography (silica gel, dichloromethane/methanol) to give N-formyl-2-amino-1-[3-(quinolin-2-ylamino)-phenyl]-ethanone (0.37 g) as a yellow powder.

m.p.: 194-196° C. (methanol) IR (KBr, cm⁻¹): 3340, 3315, 1678, 1660 Mass: 306 (m/z, (M+H)⁺) NMR (DMSO-d₆, δ): 4.72 (2H, d, J=5.6 Hz), 7.08 (1H, d, J=8.9 Hz), 7.33 (1H, ddd, J=7, 7, 1 Hz), 7.46-7.78 (5H, m), 8.10 (1H, d, J=8.9 Hz), 8.21 (1H, d, J=1.5 Hz), 8.31 (1H, d, J=7.9 Hz), 8.42 (1H, t, J=5.6 Hz), 8.70 (1H, s), 9.70 (1H, s).

### REFERENCE EXAMPLE 37

A mixture of benzalphthalide (1.0 g), ethanol (20 ml), and hydrazine hydrate (0.65 ml) was heated at reflux for 2 hours. After cooling, the reaction mixture was evaporated to dryness. The residue was recrystallized from ethanol to give 4-benzyl-phthalazin-1-one (1.00 g).

IR (nujol, cm⁻¹): 1655 NMR (DMSO-d₆, δ): 4.30 (2H, s), 7.18-7.35 (5H, m), 7.78-8.00 (3H, m), 8.24-8.29 (1H, m), 12.61 (1H, br s).

### REFERENCE EXAMPLE 38

A mixture of 4-benzyl-phthalazin-1-one (0.40 g), toluene (10 ml), and phosphorus oxychloride (1 ml) was heated at reflux for three hours. The reaction mixture was cooled and evaporated. The residues was dissolved in chloroform. The solution was washed with an aqueous saturated sodium bicarbonate solution, dried with sodium sulfate, filtered and evaporated. The residue was recrystallized from diisopropyl ether to give 1-benzyl-4-chloro-phthalazine (0.39 g) as a pale red powder.

IR (nujol, cm⁻¹): 1450 Mass: 255 (m/z, (M+H)⁺) NMR (DMSO-d₆, δ): 4.72 (2H, s), 7.15-7.45 (5H, m), 7.92-8.45 (4H, m).

### REFERENCE EXAMPLE 39

A mixture of 9-fluorenone-1-carboxylic acid (0.20 g), di(ethylene glycol) (3 ml) and hydrazine hydrate (87 μL) was heated at 130° C. for three hours and then at 180° C. for an hour. The reaction mixture was cooled and poured into water (15 ml), and then added 1 N hydrochloric acid (2 ml) thereto. The resultant precipitated were collected by filtration and washed with water to give of indeno[1,2,3-de]phthalazin-3-one (0.19 g).

IR (KBr, cm⁻¹): 3180, 3047, 1666 NMR (DMSO-d₆, δ): 7.40-7.58 (2H, m), 7.79-8.04 (4H, m), 8.24 (1H, d, J=6.2 Hz), 12.79 (1H, s).

### REFERENCE EXAMPLE 40

3-Chloro-indeno[1,2,3-de]phthalazine was prepared from indeno[1,2,3-de]phthalazin-3-one in a manner similar to Reference Example 38.

IR (KBr, cm⁻¹): 1678 NMR (DMSO-d₆, δ): 7.49-7.68 (2H, m), 7.94-8.20 (4H, m), 8.40 (1H, d, J=7 Hz).

### REFERENCE EXAMPLE 41

N-(Indeno[1,2,3-de]phthalazin-3-yl)-benzene-1,3-diamine was prepared from 3-chloro-indeno[1,2,3-de]phthalazine in a manner similar to Example 35.

IR (KBr, cm⁻¹): 3369, 1618 Mass: 311 (m/z, (M+H)⁺) NMR (DMSO-d₆, δ): 5.09 (2H, s), 6.32 (1H, d, J=8 Hz), 6.95-7.15 (2H, m), 7.39 (1H, s), 7.45-7.60 (2H, m), 7.90-8.10 (3H, m), 8.26 (1H, d, J=7 Hz), 8.46 (1H, d, J=8 Hz), 9.31 (1H, s).

### REFERENCE EXAMPLE 42

N-(Indeno[1,2,3-de]phthalazin-3-yl)-butane-1,4-diamine was prepared from 3-chloro-indeno[1,2,3-de]phthalazine (prepared as in Reference Example 40) in a manner similar to Example 35.

IR (KBr, cm⁻¹): 3369, 1618 Mass: 291 (m/z, (M+H)⁺) NMR (DMSO-d₆, δ): 1.48 (2H, q, J=7 Hz), 1.75 (2H, q, J=7 Hz), 2.61 (2H, t, J=7 Hz), 3.61 (2H, br s), 7.42-7.50 (2H, m), 7.80-8.05 (4H, m), 8.10-8.25 (2H, m), (NH₂ obscured by solvent).

### REFERENCE EXAMPLE 43

A suspension of sodium hydride (1.44 g) in dimethyl carbonate (60 ml) was added to 6,7-dihydro-1-benzothiophene-4(5H)-one (3.04 g), and the mixture was heated under reflux for an hour. After cooling, the reaction mixture was poured into IN-hydrochloric acid (100 ml) and the resulting mixture was extracted with ethyl acetate (100 ml×2). The combined extracts were dried over magnesium sulfate and filtered. After evaporation, the residue was chromatographed on a silica gel eluting with a mixture of ethyl acetate and n-hexane to give methyl 4-oxo-4,5,6,7-tetrahydro-1-benzothiophene-5-carboxylate (4.2 g) as a colorless oil.

Mass: 211 (m/z, (M+H)⁺) NMR(DMSO-d₆, δ): 2.2-2.5 (2H, m), 3.0-3.2 (2H, m), 3.67 (3H, s), 3.75 (1H, dd, J=6.4 Hz, 8.8 Hz), 7.28 (1H, d, J=5.3 Hz), 7.44 (1H, d, J=5.3 Hz).

### REFERENCE EXAMPLE 44

A mixture of methyl 4-oxo-4,5,6,7-tetrahydro-1-benzothiophene-5-carboxylate (1.0 g) and formamidine acetate (4.95 g) was heated with stirring for 40 minutes at 170° C. After cooling, the reaction mixture was poured into water (100 ml) and the resulting mixture was extracted with ethyl acetate (100 ml×2). The combined extracts were dried over magnesium sulfate, decolorized by activated charcoal powder and then filtered. After evaporation, the residue was triturated with ethyl acetate to give 5,6-dihydrothieno[2,3-h]quinazolin-4-ol (356 mg) as pale yellow crystals.

Mass: 205 (m/z, (M+H)⁺) NMR(DMSO-d₆, δ): 2.7-3.1 (4H, m), 7.3-7.5 (2H, m), 8.11 (1H, s), 12.39 (1H, br s).

### REFERENCE EXAMPLE 45

A mixture of 5,6-dihydrothieno[2,3-h]quinazolin-4-ol (204 mg), phosphorus oxychloride (767 mg) and toluene (4 ml) was heated under reflux for 2 hours. After cooling, the reaction mixture was diluted with ethyl acetate (50 ml) and washed with an aqueous saturated solution of sodium hydrogencarbonate (30 ml×2). The combined extracts were dried over magnesium sulfate and filtered. The solvent was evaporated to give 4-chloro-5,6-dihydrothieno[2,3-h]quinazoline (219 mg) as pale yellow crystals.

Mass: 223 (m/z, (M+H)⁺) NMR(DMSO-d₆, δ): 3.16 (4H, s), 7.4-7.6 (2H, m), 8.80 (1H, s).

### REFERENCE EXAMPLE 46

A suspension of sodium hydride (186 mg) in dimethyl carbonate (6.6 ml) was added to 5,6-dihydro-1-benzothiophene-7(4H)-one (394 mg), and the resulting mixture was heated under reflux for 2 hours. After cooling, the reaction mixture was poured into 0.5N-hydrochloric acid (20 ml) and the resulting mixture was extracted with ethyl acetate (30 ml×2). The combined extracts were washed with brine (30 ml), dried over magnesium sulfate and decolorized by activated charcoal powder. After filtration, the solvent was evaporated to give methyl 7-oxo-4,5,6,7-tetrahydro-1-benzothiophene-6-carboxylate (509 mg) as a yellow oil.

Mass: 211 (m/z, (M+H)⁺) NMR(DMSO-d₆, δ): 2.2-2.4 (2H, m), 2.8-3.0 (2H, m), 3.68 (3H, s), 3.80 (1H, dd, J=7.0 Hz, 8.1 Hz), 7.16 (1H, d, J=5.0 Hz), 8.06 (1H, d, J=5.0 Hz).

### REFERENCE EXAMPLE 47

A mixture of methyl 7-oxo-4,5,6,7-tetrahydro-1-benzothiophene-6-carboxylate (503 mg) and formamidine acetate (2.5 g) was heated with stirring for an hour at 180° C. After cooling, the reaction mixture was poured into water (100 ml) and the resulting mixture was extracted with ethyl acetate (5×30 ml). The combined extracts were dried over magnesium sulfate, decolorized by activated charcoal powder and then filtered. After evaporation, the residue was triturated with ethyl acetate to give 5,6-dihydrothieno[3,2-h]quinazolin-4-ol (299 mg) as pale yellow crystals.

Mass: 205 (m/z, (M+H)⁺) NMR(DMSO-d₆, δ): 2.6-3.0 (4H, m), 7.06 (1H, d, J=4.9 Hz), 7.69 (1H, d, J=4.9 Hz), 8.07 (1H, s), 12.38 (1H, br s).

### REFERENCE EXAMPLE 48

A mixture of 5,6-dihydrothieno[3,2-h]quinazolin-4-ol (250 mg), phosphorus oxychloride (938 mg) and toluene (5 ml) was heated under reflux for 7 hours. After cooling, the reaction mixture was diluted with ethyl acetate (50 ml) and washed with an aqueous saturated solution of sodium hydrogencarbonate (30 ml×2). The combined extracts were dried over magnesium sulfate and filtered. The solvent was evaporated to give 4-chloro-5,6-dihydrothieno[3,2-h]quinazoline (180 mg) as crystals.

Mass: 223 (m/z, (M+H)⁺) NMR(DMSO-d₆, δ): 2.9-3.2 (4H, m), 7.14 (1H, d, J=4.9 Hz), 7.87 (1H, d, J=4.9 Hz), 8.72 (1H, s).

### REFERENCE EXAMPLE 49

To a solution of 3-(2,3-dimethyl-3H-imidazol-4-yl)phenylamine (3.73 g) in acetone (100 ml) was added benzoyl isothiocyanate (2.68 ml), and the mixture was stirred for 8 hours at ambient temperature. Evaporation of the solvent gave 1-benzoyl-3-[3-(2,3-dimethyl-3H-imidazol-4-yl)phenyl]thiourea, which was used for further reaction without purification.

### REFERENCE EXAMPLE 50

To a solution of crude 1-benzoyl-3-[3-(2,3-dimethyl-3H-imidazol-4-yl)phenyl]thiourea in methanol (140 ml) was added a 1N aqueous solution of sodium hydroxide (25.8 ml), and the mixture was stirred for 8 hours at ambient temperature. Then, to the mixture was added 1N-hydrochloric acid (25.8 ml). After evaporation, the residue was triturated in turn with water and diisopropyl ether and dried in vacuo at 80° C. to give [3-(2,3-dimethyl-3H-imidazol-4-yl)phenyl]thiourea (2.23 g).

APCI-mass: 247 (m/z, [M+H]⁺) NMR(DMSO-d₆, δ): 2.34(3H, s), 3.55(3H, s), 6.87(1H, s), 7.04-7.95(6H, m), 9.77(1H, s).

### REFERENCE EXAMPLE 51

To a solution of 3-(4,5-dimethylimidazol-1-yl)phenylamine (1.5 g) in acetone (40 ml) was added N-benzoyl isothiocyanate (1.08 ml) at ambient temperature. After stirring for 8 hours, the resultant precipitate was collected by filtration, washed in turn with acetone and diisopropyl ether, and dried in vacuo to give 1-benzoyl-3-[3-(4,5-dimethylimidazol-1-yl)phenyl]thiourea (1.80 g).

APCI-mass: 350.67 (m/z, (M+H)⁺) NMR(DMSO-d₆, δ): 2.11(3H, s), 2.14(3H, s), 7.25-7.39(1H, m), 7.45-7.73(6H, m), 7.92(1H, s), 7.98(2H, d, J=8.6 Hz), 11.68(1H, brs), 12.69(1H, brs).

### REFERENCE EXAMPLE 52

To a solution of 1-benzoyl-3-[3-(4,5-dimethylimidazol-1-yl)phenyl]thiourea (1.60 g) in methanol (30 ml) was added a 1N aqueous solution of sodium hydroxide (5.94 ml) at ambient temperature. After stirring for 8 hours at ambient temperature, 1N-hydrochloric acid (5.94 ml) was added to the mixture . After evaporation, the residue was triturated with diisopropyl ether. The resulting powders were collected by filtration and washed in turn with water and diisopropyl ether to give [3-(4,5-dimethylimidazol-1-yl)phenylthiourea (1.25 g).

APCI-mass: 247.13 (m/z, (M+H)⁺) NMR(DMSO-d₆, δ): 2.00(6H, s), 7.08-7.20(1H, m), 7.30-7.80(5H, m), 7.95(1H, d, J=8.6 Hz), 10.00(1H, s).

### REFERENCE EXAMPLE 53

To a solution of [6-(2-methylpyridin-3-yloxy)pyridin-3-yl]amine (0.5 g) in acetone (10 ml) was added benzoyl isothiocyanate (680 mg) at ambient temperature. After stirring for 4 hours at ambient temperature, the reaction mixture was evaporated under reduced pressure to give crude 1-benzoyl-3-[6-(2-methylpyridin-3-yloxy)pyridin-3-yl]thiourea, which was used for the next step without further purification.

### REFERENCE EXAMPLE 54

To a solution of crude 1-benzoyl-3-[6-(2-methylpyridin-3-yloxy)pyridin-3-yl]thiourea (1.52 g) in methanol (30 ml) was added a 1N aqueous solution of sodium hydroxide (5.42 ml) at ambient temperature. After stirring for 12 hours, to the mixture was added 1N-hydrochloric acid (5.42 ml), and the resulting mixture was evaporated under reduced pressure. The residue was chromatographed on silica gel eluting with a mixture of dichloromethane and methanol (0-10% V/V) to give [6-(2-methylpyridin-3-yloxy)pyridin-3-yl]thiourea (0.479 g).

APCI-mass: 261.07 (m/z, (M+H)⁺) NMR(DMSO-d₆, δ): 2.33(3H, s), 7.10(1H, d, J=8.7 Hz), 7.29-7.41(1H, m), 7.49(1H, d, J=8.7 Hz), 7.88-8.04(2H, m), 8.35(1H, d, J=4.7 Hz), 9.61(1H, s).

### REFERENCE EXAMPLE 55

To a solution of (3-amino-5-chlorophenyl)carbamic acid tert-butyl ester (1 g) in acetone (20 ml) was added benzoyl isothiocyanate (672 mg) at ambient temperature. After stirring for an hour at ambient temperature, the precipitate was collected by filtration and washed with acetone to give [3-(3-benzoylthioureido)-5-chlorophenyl]carbamic acid tert-butyl ester (0.752 g). Concentration of the mother liquid gave a second crop (0.774 g).

### REFERENCE EXAMPLE 56

To a solution of crude [3-(3-benzoylthioureido)-5-chlorophenyl]carbamic acid tert-butyl ester (1.51 g) in methanol (30 ml) was added a 1N aqueous solution of sodium hydroxide (4.84 ml) at ambient temperature. After stirring for 12 hours, to the mixture was added 1N-hydrochloric acid (4.84 ml), and the mixture was evaporated under reduced pressure. The residue was dissolved in diisopropyl ether and the resultant precipitate was removed by filtration. Evaporation of the solvent under reduced pressure gave (3-chloro-5-thioureidophenyl)carbamic acid tert-butyl ester (1.61 g).

APCI-mass: 300.67, 302.53 (m/z, (M−H)⁺)

### REFERENCE EXAMPLE 57

To a solution of 2-indanone (0.50 g) in dichloromethane (0.2 ml) was added sulfuryl chloride (0.378 ml) at ambient temperature. After stirring for 12 hours at ambient temperature, the reaction mixture was diluted with a mixture of ethyl acetate and water, adjusted at around pH7 with an aqueous potassium carbonate solution. The separated organic layer was dried over magnesium sulfate and evaporated in vacuo. To the solution of the residue in ethanol (2 ml) was added (3-chloro-5-thioureidophenyl)carbamic acid tert-butyl ester (343 mg), and the resulting mixture was heated for an hour at 100° C. To the reaction mixture was added a 4N solution of hydrogen chloride in dioxane (1 ml), and heating was continued for 1.5 hours. After cooling to ambient temperature, the resultant precipitate was collected by filtration and washed in turn with ethanol and diisopropyl ether to give 5-chloro-N-(4H-indeno[2,1-d][1,3]thiazol-2-yl)benzene-1,3-diamine hydrochloride (0.183 g).

APCI-mass: 314.27, 316.20 (m/z, free form of (M+H)⁺) NMR(DMSO-d₆, δ): 3.77(2H, s), 6.78(1H, s), 7.14(1H, dt, J=1.3, 7.4 Hz), 7.29(1H, t, J=7.4 Hz), 7.36-7.55(3H, m), 7.62(1H, s), 10.96(1H, s).

### REFERENCE EXAMPLE 58

To a solution of 4,6-dichloropyrimidine (0.29 g) in a mixture of dimethoxyethane(6.5 ml) and a 2M aqueous sodium carbonate solution (3.3 ml) were added phenylboronic acid (0.36 g) and tetrakis(triphenylphosphine)palladium(0) (0.11 g) under nitrogen atmosphere, and the mixture was heated for 3 hours at 100° C. After cooling to ambient temperature, the separated organic layer was evaporated under reduced pressure. The residue was taken up into ethyl acetate, washed in turn with a 10% aqueous potassium carbonate solution and brine, and dried over sodium sulfate. After evaporation, the residue was chromatographed on silica gel eluting with 0%-6% ethyl acetate in n-hexane to give 4-chloro-6-phenylpyrimidine (0.13 g).

APCI-mass: 191 (m/z, [M+H]⁺) NMR(DMSO-d₆, δ): 7.48-7.70(3H, m), 8.23-8.31(2H, m), 8.33(1H, s), 9.10(1H, s).

### REFERENCE EXAMPLE 59

To a solution of 4,6-dichloropyrimidine (0.34 g) in a mixture of dimethoxyethane (7.5 ml) and a 2M aqueous sodium carbonate solution (3.8 ml) were added thiophene-2-boronic acid (0.44 g) and tetrakis(triphenylphosphine)palladium(0) (0.13 g) under nitrogen atmosphere, and the mixture was heated for an hour at 90° C. After cooling to ambient temperature, the separated organic layer was evaporated under reduced pressure. The residue was taken up into ethyl acetate, washed in turn with a 10% aqueous potassium carbonate solution and brine, and dried over sodium sulfate. After evaporation, the residue was chromatographed on silica gel eluting with 0%-6% ethyl acetate in n-hexane to give 4-chloro-6-(thiophen-2-yl)pyrimidine (0.24 g).

NMR(DMSO-d₆, δ): 7.28(1H, dd, J=3.9, 5.0 Hz), 7.92(1H, dd, J=1.0, 5.0 Hz), 8.20(1H, dd, J=1.0, 3.9 Hz), 8.27(1H, d, J=1.1 Hz), 8.94(1H, d, J=1.1 Hz).

### REFERENCE EXAMPLE 60

To a mixture of 3-(2,3-dimethyl-3H-imidazol-4-yl)phenylamine (2.57 g) and bis(tert-butoxycarbonyl)thiourea (4.59 g) in dichloromethane (50 ml) were added triethylamine (4.2 ml) and 2-chloro-1-methylpyridinium iodide (4.21 g). The resultant mixture was stirred for 12 hours at ambient temperature, and taken up into a mixture of ethyl acetate and water. The separated organic layer was washed in turn with water and an aqueous sodium hydrogencarbonate solution, and dried over magnesium sulfate. Evaporation of the solvent gave N, N′-bis(tert-butoxycarbonyl)-N″-[3-(2,3-dimethyl-3H-imidazol-4-yl)phenyl]guanidine (5.18 g).

NMR(DMSO-d₆, δ): 1.10-1.80(18H, m), 2.35(3H, s), 3.58(3H, s), 6.90(1H, s), 7.13-7.24(1H, m), 7.35-7.45(2H, m), 7.80(1H, s), 10.03(1H, s), 11.39(1H, s).

### REFERENCE EXAMPLE 61

To a solution of N,N′-bis(tert-butoxycarbonyl)-N″-[3-(2,3-dimethyl-3H-imidazol-4-yl)phenyl]guanidine (5.01 g) was added a 4N solution of hydrogen chloride in dioxane (100 ml), and the mixture was stirred for 8 hours at ambient temperature. After evaporation of the solvents, the residue was added with an excess amount of hydrogen chloride gas to give N-[3-(2,3-dimethyl-3H-imidazol-4-yl)phenyl]guanidine dihydrochloride.

APCI-mass: 230 (m/z, [M+H]⁺, as free form)

### REFERENCE EXAMPLE 62

A mixture of 7-methoxy-1-oxo-1,2,3,4-tetrahydronaphthalene-2-carboxylic acid methyl ester (9.69 g) and formamidine acetate (17.2 g) was heated for 30 minutes at 180° C. After cooling to ambient temperature, to the mixture were added water (20 ml) and ethyl acetate (10 ml). The resultant precipitate was collected by filtration, washed with small portions of ethyl acetate and water and dried under reduced pressure to give 9-methoxy-5,6-dihydrobenzo[h]quinazolin-4-ol (2.75 g).

APCI-mass: 229.20 (m/z, (M+H)⁺) NMR(DMSO-d₆, δ): 2.53-2.70(2H, m), 2.70-2.86(2H, m), 3.78 (3H, s), 6.94(1H, dd, J=2.8, 8.3 Hz), 7.20(1H, d, J=8.3 Hz), 7.60(1H, d, J=2.8 Hz), 8.17(1H, s).

### REFERENCE EXAMPLE 63

To a suspension of 9-methoxy-5,6-dihydrobenzo[h]quinazolin-4-ol (2.44 g) in toluene (10 ml) was added phosphorous oxychloride (10 ml), and the mixture was heated for 4 hours at 110° C. After evaporation of the solvent under reduced pressure, the residue was taken up into a mixture of ethyl acetate and water, and pH of the mixture was adjusted to 7.5 with an aqueous potassium carbonate solution. The separated organic layer was washed with brine and dried over magnesium sulfate. After evaporation, the residue was triturated with diisopropyl ether to give 4-chloro-9-methoxy-5,6-dihydrobenzo[h]quinazoline (1.92 g).

APCI-mass: 247.27 (m/z, (M+H)⁺) NMR(DMSO-d₆, δ): 2.83-3.10(4H, m), 3.82(3H, s), 7.09(1H, dd, J=2.8,8.4 Hz), 7.30(1H, d, J=8.4 Hz), 7.75(1H, d, J=2.8 Hz), 8.92(1H, s).

### REFERENCE EXAMPLE 64

To a suspension of ethyl 7-methyl-5-oxo-2,3,4,5-tetrahydro-1-benzoxepine-4-carboxylate (1.02 g) in ethanol (10 ml) was added hydroxylamine hydrochloride (0.86 g), and the mixture was refluxed for 18 hours. The mixture was diluted with ethyl acetate and washed with water and brine. The separated organic layer was dried over magnesium sulfate and evaporated. The residue was triturated with diisopropyl ether, collected by filtration and dried under reduced pressure to give 9-methyl-4,5-dihydro[1]benzoxepino[5,4-c]isoxazol-3-ol (636 mg, 71.2%).

APCI-mass: 218 (m/z, (M+H)⁺) NMR(DMSO-d₆, δ): 2.30 (3H, s), 2.68 (2H, t, J=5.1 Hz), 4.23 (2H, t, J=5.1 Hz), 7.02 (1H, d, J=8.3 Hz), 7.28 (1H, d, J=8.3 Hz), 7.34 (1H, s), 11.96 (1H, broad s).

### REFERENCE EXAMPLE 65

A suspension of 9-methyl-4,5-dihydro[1]benzoxepino[5,4-c]isoxazol-3-ol (186 mg) in phosphorus oxychloride (2 ml) was refluxed for an hour. The mixture was poured onto a mixture of crushed ice and ethyl acetate, and the resulting mixture was stirred for an hour. The separated organic layer was washed with an aqueous saturated solution of sodium hydrogencarbonate and brine, dried over magnesium sulfate and evaporated to give 3-chloro-9-methyl-4,5-dihydro[1]benzoxepino[5,4-c]isoxazole (180 mg, 89.1%).

APCI-mass: 236 (m/z, (M+H)⁺) NMR(DMSO-d₆, δ): 2.31(3H, s), 2.93 (2H, t, J=5.3 Hz), 4.26 (2H, t, J=5.3 Hz), 7.00 (1H, d, J=8.3 Hz), 7.24 (1H, dd, J=8.3 Hz, 2.2 Hz), 7.89 (1H, d, J=2.2 Hz).

### REFERENCE EXAMPLE 66

A mixture of cycloheptanone (951 mg) and N,N-dimethylformamide dimethylacetal was stirred for 6 hours at 130° C. The mixture was evaporated under reduced pressure to give 2-((dimethylamino)methylene)cycloheptanone (360 mg).

APCI-MASS: 168 (m/z, (M+H)⁺) NMR(DMSO-d₆, δ): 1.4-1.8 (6H, m), 2.3-2.7 (4H, m), 2.98 (6H, s), 7.19 (3H, s).

### REFERENCE EXAMPLE 67

To a solution of 2-indanone (264 mg) in tetrahydrofuran (2 ml) was added N,N-dimethylformamide dimethylacetal (0.29 ml). The mixture was stirred for an hour at ambient temperature and evaporated to give 1-[(dimethylamino)methylene]-1,3-dihydro-2H-inden-2-one (374 mg, 100%).

APCI-Mass: 188.2 (m/z, (M+H)⁺) NMR(DMSO-d₆, δ): 3.0-3.4 (8H, m), 6.8-7.4 (4H, m), 7.54 (1H, s).

### REFERENCE EXAMPLE 68

A suspension of 5-oxo-2,3,4,5-tetrahydro-benzo[b]oxepine-4-carbonitrile (0.75 g), hydroxylamine hydrochloride (835 mg) and sodium acetate (1.64 g) in a mixture of ethanol (15 ml) and water (5 ml) was stirred for 24 hours at 60° C. and concentrated under reduced pressure. The residue was suspended in ethyl acetate and the resulting mixture was washed with water and brine. The separated organic layer was dried over magnesium sulfate and evaporated. The residue was purified by a silica gel column chromatography eluting with 20-40% ethyl acetate in n-hexane to give 4,5-dihydro[1]benzoxepino[5,4-c]isoxazol3-amine (428 mg, 52.8%).

APCI-Mass: 203 (m/z, (M+H)⁺) NMR(DMSO-d₆, δ): 2.69 (2H, t, J=5.2 Hz), 4.22 (2H, t, J=5.2 Hz), 6.65 (2H, broad s), 7.0-7.2 (2H, m), 7.31 (1H, dt, J=1.8 Hz, 7.6 Hz), 8.00 (1H, dd, J=1.7 Hz, 7.7 Hz).

### REFERENCE EXAMPLE 69

To a suspension of 1,2-dimethylimidazole (2.0 g), 1,3-dibromobenzene (14.72 g) and potassium carbonate (6.0 g) in N,N-dimethylformamide (80 ml) was added palladium acetate (234 mg), and the mixture was stirred under nitrogen atmosphere for 6 hours at 140° C. The mixture was concentrated under reduced pressure. To the residue was added ethyl acetate and water. The organic layer was separated and washed with water and brine. The organic layer was dried over magnesium sulfate and evaporated. The residue was purified by a silica gel column chromatography eluting with 1-2% methanol in dichloromethane to give 1-bromo-3-(1,2-dimethylimidazol-5-yl)benzene (261 mg, 5%).

APCI-Mass: 251 (m/z, (M+H)⁺) NMR(DMSO-d₆, δ): 2.34 (3H, s), 3.53 (3H, s), 6.94 (1H, s), 7.3-7.7 (4H, m).

### REFERENCE EXAMPLE 70

A mixture of ethyl 5-oxo-2,3,4,5-tetrahydro-1-benzoxepine-4-carboxylate (469 mg) and formamidine acetate (1.0 g) was heated for 50 minutes at 175° C. (all dissolved). After cooling, to the mixture were added ethyl acetate (100 ml), water (100 ml) and 3N-hydrochloric acid (5 ml). The separated organic layer was washed with water (twice) and brine, dried over magnesium sulfate. The mixture was filtered and evaporated. The residue was recrystallized from methanol to provide 5,6-dihydro[1]benzoxepino[5,4-d]pyrimidin-4-ol (162 mg) as white crystals.

mp 241-243° C. Mass: 215 (m/z, (M+H)⁺) NMR(DMSO-d₆, δ): 2.78 (2H, t, J=5.7 Hz), 4.44 (2H, t, J=5.7 Hz), 7.08 (1H, dd, J=8.0, 1.2 Hz), 7.20 (1H, ddd, J=7.9, 7.9, 1.3 Hz), 7.42 (1H, ddd, J=7.4, 7.4, 1.8 Hz), 8.01 (1H, dd, J=7.9, 1.8 Hz), 8.20 (1H, s).

### REFERENCE EXAMPLE 71

A mixture of 5,6-dihydro[1]benzoxepino[5,4-d]pyrimidin-4-ol (150 mg) and phosphorus oxychloride (1 ml) was heated under reflux for two hours. After cooling, the mixtures was carefully poured into a mixture of ice and water, and the resulting mixture was neutralized with an aqueous potassium carbonate solution until basic. The resultant precipitate was collected, washed with water and air-dried overnight to give 4-chloro-5,6-dihydro[1]benzoxepino[5,4-d]pyrimidine as white crystals.

mp 114-115° C. Mass: 233 (m/z, (M+H)⁺) NMR(DMSO-d₆, δ): 3.08 (2H, t, J=5.9 Hz), 4.57 (2H, t, J=5.9 Hz), 7.19 (1H, dd, J=8.0, 1.2 Hz), 7.32 (1H, ddd, J=7.6, 7.6, 1.2 Hz), 7.56 (1H, ddd, J=7.7, 7.7, 1.8 Hz), 7.99 (1H, dd, J=7.9, 1.8 Hz), 9.02 (1H, s).

### REFERENCE EXAMPLE 72

To a mixture of 3-(imidazol-1-yl)aniline (0.20 g) and formic acid (2 ml) at room temperature was added acetic anhydride (0.13 ml). After stirring for two hours at room temperature, the solution was evaporated. The residue was dissolved in ethyl acetate and washed with an aqueous saturated solution of sodium bicarbonate (three times). The organic layer was dried over sodium sulfate, filtered and evaporated. The residue was recrystallized from diisopropyl ether to N-formyl-3-(imidazol-1-yl)aniline (0.23 g).

IR (nujol, cm⁻¹): 3100, 1685 NMR(CDCl₃, δ): 7.10-7.49 (5H, m), 7.88-7.95 (2H, m), 8.29 (1H, br s), 8.45. (1H, s).

### REFERENCE EXAMPLE 73

To a solution of N-formyl-3-(imidazol-1-yl)aniline (100 mg) in dimethylformamide (5 ml) at 5° C. was added sodium hydride (25 mg). After stirring for 10 minutes, 2-chloro-5-nitropyridine (0.11 g) was added to the reaction mixture, and the mixture was stirred for 24 hours at room temperature. After adding water and ethyl acetate to the reaction mixture, the organic layer was separated and washed with brine. The organic layer was dried over sodium sulfate, filtered and evaporated. The residue was recrystallized from methanol to give N-[3-(imidazol-1-yl)phenyl]-N-(5-nitropyridin-2-yl)-formamide (60 mg).

IR (KBr, cm⁻¹): 1651 Mass: 282 (m/z, (M—CHO+H)⁺) NMR(DMSO-d₆, δ): 6.97 (1H, d, J=9 Hz), 7.13 (1H, s), 7.33 (1H, d, J=8 Hz), 7.50 (1H, t, J=8 Hz), 7.62 (1H, d, J=8 Hz), 7.71 (1H, s), 8.08 (1H, s), 8.22 (1H, s), 8.34 (1H, dd, J=9, 3 Hz), 9.11 (1H, d, J=2 Hz), 10.32 (1H, s).

### REFERENCE EXAMPLE 74

To N-[3-(imidazol-1-yl)phenyl]-N-(5-nitropyridin-2-yl)-formamide (50 mg) in methanol (5 ml) was added 10% palladium on carbon (10 mg). The mixture was stirred under an atmosphere of hydrogen gas for five hours, filtered through Celite and evaporated. To the reaction mixture, added were acetic acid (5 ml) and then 2,5-dimethoxytetrahydrofuran (31 μl). The mixture was heated under reflux for an hour. After evaporation, the residue was dissolved in ethyl acetate. The solution was washed with an aqueous saturated solution of sodium bicarbonate, dried over sodium sulfate, filtered and evaporated. The residue was purified by a silica gel column chromatography eluting with a mixture of chloroform and methanol to give N-[3-(imidazol-1-yl)phenyl]-N-[5-(pyrrol-1-yl)pyridin-2-yl]-formamide (50 mg) as an oil.

Mass: 302 (m/z, (M—CHO+H)⁺)

### REFERENCE EXAMPLE 75

To a solution of 5-chloro-1,3-benzenediamine (7.48 g) in tetrahydrofuran (50 ml) was added slowly a 1.5M solution of n-butyl lithium in n-hexane (27.3 ml) at 0° C. The resultant mixture was stirred for 30 minutes at 0° C. To the mixture was added a solution of 3-chloro-6-fluorobenzo[d]isoxazole (1.8 g) in tetrahydrofuran (5 ml). After stirring for 15 minutes at 0° C. and for an hour at ambient temperature, the reaction mixture was poured into a mixture of water and ethyl acetate. The separated organic layer was washed well with 1N-hydrochloric acid and dried over potassium carbonate. After evaporation under reduced pressure, the residue was crystallized from methanol to give 5-chloro-N-(6-fluorobenzo[d]isoxazol-3-yl)benzene-1,3-diamine (1.54 g).

APCI-Mass: 278 (m/z, (M+H)⁺) NMR(DMSO-d₆, δ): 5.50(2H, s), 6.24(1H, t, J=3.7 Hz), 6.88(2H, t, J=1.9 Hz), 7.27(1H, dt, J=2.1, 9.0 Hz), 7.57(1H, dd, J=2.1, 9.0 Hz), 8.09-8.22(1H, m), 9.47(1H, s).

### REFERENCE EXAMPLE 76

To a solution of 5-chloro-1,3-benzenediamine (1.5 g) in tetrahydrofuran (30 ml) was added slowly a 1.5M solution of n-butyl lithium in n-hexane (5.61 ml) at 0° C. The resultant mixture was stirred for 30 minutes at 0° C. To the mixture was added a solution of 2,6-dichlorobenzothiazole (429 mg) in tetrahydrofuran (5 ml). After stirring for 15 minutes at 0° C. and for an hour at ambient temperature, the reaction mixture was poured into a mixture of water and ethyl acetate. The separated organic layer was washed well with 0.1N-hydrochloric acid (total 400 ml). After evaporation under reduced pressure, the residue was crystallized from methanol to give 5-chloro-N-(6-chlorobenzothiazol-2-yl)-benzene-1,3-diamine (171 mg).

APCI-Mass: 312.20, 310.27 (m/z, (M+H)⁺) NMR(DMSO-d₆, δ): 5.50(2H, s), 6.28(1H, t, J=1.9 Hz), 6.81(1H, t, J=1.9 Hz), 7.07(1H, t, J=1.9 Hz), 7.33(1H, dd, J=2.2, 8.6 Hz), 7.56(1H, d, J=8.6 Hz), 7.94(1H, d, J=2.2 Hz), 10.41(1H, s).

### REFERENCE EXAMPLE 77

To a solution of 5-chloro-1,3-benzenediamine (1.43 g) in tetrahydrofuran (10 ml) under nitrogen atmosphere at 0° C. was added a 1.54 M solution of n-butyl lithium in n-hexane (5.8 ml) dropwise. After a precipitate was appeared, the mixture was stirred for 30 minutes. Then to the reaction mixture was added 3-chloro-1,2-benzo[d]isoxazole (0.77 g) all at once. The reaction mixture was stirred for an hour at 0° C. and then for an hour at room temperature (all were dissolved to give a clear, black solution).

After adding water (10 ml) dropwise, then ethyl acetate (100 ml) and water (100 ml) to the reaction mixture, the organic phase was separated. The organic phase was washed with dilute hydrochloric acid (three times), an aqueous saturated solution of sodium bicarbonate (twice) and brine. The organic phase was dried over magnesium sulfate, filtered and evaporated.

The residue was purified by a silica gel column chromatography eluting with a mixture of dichloromethane and methanol, followed by recrystallization from dichloromethane to give N¹-(1,2-benzo[d]isoxazol-3-yl)-5-chloro-1,3-benzenediamine (0.63 g) as green crystals.

mp 192-194° C. Mass: 260 (m/z, (M+H)⁺) NMR(DMSO-d₆, δ): 5.49 (2H, s), 6.23 (1H, dd, J=1.8, 1.8 Hz), 6.92 (2H, s), 7.32-7.41 (1H, m), 7.58-7.70 (2H, m), 8.13 (1H. d, J=7.9 Hz), 9.42 (1H, s).

### REFERENCE EXAMPLE 78

N¹-(1,2-benzo[d]isoxazol-3-yl)-5-(trifluoromethyl)-1,3-benzenediamine as white crystals was obtained in a similar manner to Reference Example 77.

mp 197-198° C. Mass: 294 (m/z, (M+H)⁺) NMR(DMSO-d₆, δ): 5.68 (2H, s), 6.49 (1H, s), 7.15 (1H, s), 7.24 (1H, s), 7.33-7.43 (1H, m), 7.56-7.69 (2H, m), 8.14 (1H, d, J=7.9 Hz), 9.58 (1H, s).

### REFERENCE EXAMPLE 79

To a suspension of 3-bromo-2-fluorobenzoic acid (1.16 g) in dichloromethane (10 ml) were added oxalyl chloride (1.34 g) and N,N-dimethylformamide (1 drop) under stirring at ambient temperature. After stirring for 2 hours, the reaction mixture was evaporated in vacuo, and the residue was taken up into dichloromethane (5 ml) to give a solution of a crude acid chloride. To a solution of 3-(1,2-dimethyl-1H-imidazol-5-yl)aniline (900 mg) and triethylamine (971 mg) in dichloromethane (10 ml) was added the solution of the acid chloride dropwise under stirring at ambient temperature. After stirring for 14 hours, the reaction mixture was evaporated. The residue was diluted with water (100 ml) and extracted with ethyl acetate (50 ml×2). The combined extracts were washed with an aqueous saturated solution of ammonium chloride (50 ml×2), an aqueous saturated solution of sodium hydrogencarbonate (50 ml×2) and brine (50 ml). The organic layer was dried over magnesium sulfate and filtered. After evaporation, the residue was chromatographed on silica gel diluting with a mixture of dichloromethane and methanol to give 3-bromo-N-[3-(1,2-dimethyl-1H-imidazol-5-yl)phenyl]-2-fluorobenzamide (1.66 g) as crystals.

Mass: 388,390 (1:1 ratio, Br isotopes, m/z, (M+H)⁺) NMR(DMSO-d₆, δ): 2.35 (3H, s), 3.55 (3H, s), 6.87 (1H, s), 7.19 (1H, d, J=7.8 Hz), 7.31 (1H, t, J=7.8 Hz), 7.44 (1H, t, J=7.8 Hz), 7.6-7.8 (3H, m), 7.8-8.0 (1H, m).

### REFERENCE EXAMPLE 80

The following compounds described in (1) and (2) were obtained in a manner similar to Reference Example 79.

(1) N-[3-(1,2-Dimethyl-1H-imidazol-5-yl)phenyl]-2-fluoro-3-(3-thienyl)benzamide

Mass: 392 (m/z, (M+H)⁺) NMR(DMSO-d₆, δ): 2.36 (3H, s), 3.55 (3H, s), 6.87 (1H, s), 7.18 (1H, d, J=7.8 Hz), 7.3-8.0 (9H, m), 10.60 (1H, br s).

(2) N-[3-(1,2-Dimethyl-1H-imidazol-5-yl)phenyl]-2-fluoro-3-(2-thienyl)benzamide

Mass: 392 (m/z, (M+H)⁺) NMR(DMSO-d₆, δ): 2.36 (3H, s), 3.55 (3H, s), 6.88 (1H, s), 7.1-7.3 (2H, m), 7.3-7.5 (2H, m), 7.5-7.9 (5H, m), 7.9-8.1 (1H, m), 10.65 (1H, br s).

### REFERENCE EXAMPLE 81

To a suspension of 3-bromo-N-[3-(1,2-dimethyl-1H-imidazol-5-yl)phenyl]-2-fluorobenzamide (1.94 g) in dichloromethane (20 ml) was added phosphorus pentachloride (1.25 g) under stirring at ambient temperature. The mixture was heated under reflux for 2 hours. After cooling, the reaction mixture was evaporated in vacuo, and the residue was washed with n-hexane (5×60 ml). The resultant powder was taken up into tetrahydrofuran (30 ml) to give a solution of a crude iminochloride compound. To the solution was added O-(trimethylsilyl)hydroxylamine (1.28 g) dropwise at 0° C. After stirring for 88 hours at ambient temperature, the reaction mixture was evaporated in vacuo. The resultant residue was dissolved in ethyl acetate (400 ml) and washed with an aqueous saturated solution of sodium hydrogencarbonate (300 ml), water (2×300 ml) and brine (300 ml). The organic layer was dried over magnesium sulfate and filtered. After evaporation, the residue was chromatographed on a silica gel eluting with a mixture of dichloromethane and methanol. The residue was triturated with ethyl acetate to give 3-bromo-N-[3-(1,2-dimethyl-1H-imidazol-5-yl)phenyl]-2-fluoro-N′-hydroxybenzenecarboximidamide (592 mg) as crystals.

Mass: 403, 405 (1:1 ratio, Br isotopes, m/z, (M+H)⁺) NMR(DMSO-d₆, δ): 2.28 (3H, s), 3.25 (3H, s), 6.55 (1H, s), 6.61 (1H, br s), 6.75 (1H, d, J=8.0 Hz), 6.87 (1H, d, J=7.7 Hz), 7.1-7.3 (2H, m), 7.4-7.6 (1H, m), 7.7-7.9 (1H, m), 8.73 (1H, br s), 10.71 (1H, s).

### REFERENCE EXAMPLE 82

The following compounds described in (1) to (3) were obtained in a manner similar to Reference Example 81.

(1) N-[3-(1,2-Dimethyl-1H-imidazol-5-yl)phenyl]-2-fluoro-N′-hydroxy-3-(2-thienyl)benzenecarboximidamide

Mass: 407 (m/z, (M+H)⁺) NMR(DMSO-d₆, δ): 2.25 (3H, s), 3.22 (3H, s), 6.51 (1H, s), 6.65 (1H, br s), 6.7-6.9 (2H, m), 7.1-7.2 (2H, m), 7.31 (1H, t, J=7.7 Hz), 7.4-7.6 (2H, m), 7.6-7.7 (1H, m), 7.7-7.9 (1H, m), 8.70 (1H, br s), 10.63 (1H, s).

(2) N-[3-(1,2-Dimethyl-1H-imidazol-5-yl)phenyl]-2-fluoro-N′-hydroxy-3-(3-thienyl)benzenecarboximidamide

Mass: 407 (m/z, (M+H)⁺) NMR(DMSO-d₆, δ): 2.25 (3H, s), 3.20 (3H, s), 6.51 (1H, s), 6.63 (1H, br s), 6.7-6.9 (2H, m), 7.15 (1H, t, J=7.8 Hz), 7.2-7.9 (6H, m), 8.68 (1H, br s), 10.59 (1H, s).

(3) 3-Bromo-N-[3-(1,2-dimethyl-1H-imidazol-5-yl)phenyl]-2-fluorobenzenecarbohydrazonamide

Mass: 402, 404 (1:1 ratio, Br isotopes, m/z, (M+H)⁺) NMR(DMSO-d₆, δ): 2.29 (3H, s), 3.30 (3H, s), 6.21 (2H, br s), 6.4-6.5 (1H, m), 6.5-6.7 (2H, m), 6.78 (1H, d, J=7.7 Hz), 7.0-7.3 (2H, m), 7.4-7.7 (2H, m).

### REFERENCE EXAMPLE 83

To a mixture of methyl 3-bromo-2-fluorobenzoate (117 mg), 2-thiopheneboronic acid (83 mg) and 1,2-dimethoxyethane (2 ml) were added a 2M aqueous solution of sodium carbonate (0.83 ml) and tetrakis(triphenylphosphine)palladium(0) (29 mg) at ambient temperature. The mixture was heated for 3 hours at 90° C. After cooling, the reaction mixture was diluted with ethyl acetate (30 ml), and washed with water (20 ml×3) and brine (20 ml). The organic layer was dried over magnesium sulfate and filtered. After evaporation, the residue was chromatographed on a silica gel eluting with a mixture of ethyl acetate and n-hexane to give methyl 2-fluoro-3-(2-thienyl)benzoate (99 mg).

Mass: 237 (m/z, (M+H)⁺) NMR(DMSO-d₆, δ): 3.89 (3H, s), 7.1-7.3 (1H, m), 7.39 (1H, t, J=7.8 Hz), 7.6-7.9 (3H, m), 8.0-8.2 (1H, m).

### REFERENCE EXAMPLE 84

Methyl 2-fluoro-3-(3-thienyl)benzoate was obtained in a manner similar to Reference Example 83.

Mass: 237 (m/z, (M+H)⁺) NMR(DMSO-d₆, δ): 3.88 (3H, s), 7.38 (1H, t, J=7.7 Hz), 7.4-7.6 (1H, m), 7.6-7.9 (2H, m), 7.9-8.1 (2H, m).

### REFERENCE EXAMPLE 85

To a solution of methyl 2-fluoro-3-(2-thienyl)benzoate-(71 mg) in methanol (2 ml) was added a 1N aqueous solution of sodium hydroxide (0.9 ml) at 0° C. under stirring. After stirring for an hour at ambient temperature, the reaction mixture was acidified with 1N-hydrochloric acid, diluted with ethyl acetate (30 ml), and then washed with water (30 ml×2) and brine (20 ml). The organic layer was dried over magnesium sulfate, filtered, and evaporated to give 2-fluoro-3-(2-thienyl)benzoic acid (66 mg) as colorless crystals.

Mass: 221 (m/z, (M−H)⁺) NMR(DMSO-d₆, δ): 7.1-7.3 (1H, m), 7.36 (1H, t, J=7.7 Hz), 7.6-7.9 (3H, m), 7.9-8.1 (1H, m), 13.42 (1H, br s).

### REFERENCE EXAMPLE 86

2-Fluoro-3-(3-thienyl)benzoic acid was obtained in a manner similar to Reference Example 85.

Mass: 221 (m/z, (M−H)⁺) NMR(DMSO-d₆, δ): 7.34 (1H, t, J=7.7 Hz), 7.4-7.6 (1H, m), 7.6-8.0 (4H, m), 13.32 (1H, br s).

### REFERENCE EXAMPLE 87

To a mixture of 1,1′-thiocarbonyldiimidazole (535 mg) and acetonitrile (7 ml) was added a solution of 3-(1,2-dimethyl-1H-imidazol-5-yl)aniline (375 mg) in acetonitrile (7 ml) dropwise over period of 15 minutes under stirring at 0° C. After stirring for 2 hours at ambient temperature, 2-(aminomethyl)pyridine (433 mg) was added to the mixture, and the reaction mixture was heated for 4 hours at 50-70° C. After cooling, the reaction mixture was evaporated. The residue was chromatographed on a silica gel eluting with a mixture of dichloromethane and methanol to give N-[3-(1,2-dimethyl-1H-imidazol-5-yl)phenyl]-N′-(2-pyridylmethyl)thiourea (523 mg).

Mass: 338 (m/z, (M+H)⁺) NMR(DMSO-d₆, δ): 2.34 (3H, s), 3.55 (3H, s), 4.84 (2H, d, J=5.1 Hz), 6.88 (1H, s), 7.1-7.5 (5H, m), 7.64 (1H, br s), 7.7-7.9 (1H, m), 8.39 (1H, t, J=5.1 Hz), 8.53 (1H, d, J=4.7 Hz), 9.98 (1H, br s).

### REFERENCE EXAMPLE 88

N-{[3-Chloro-5-(trifluoromethyl)-2-pyridyl]methyl}-N′-[3-(1,2-dimethyl-1H-imidazol-5-yl)phenyl]thiourea was obtained in a manner similar to Reference Example 87.

Mass: 440 (m/z, (M+H)⁺) NMR(DMSO-d₆, δ): 2.35 (3H, s), 3.56 (3H, s), 5.03 (2H, d, J=4.4 Hz), 6.88 (1H, s), 7.1-7.3 (1H, m), 7.3-7.5 (2H, m), 7.68 (1H, br s), 8.41 (1H, t, J=4.4 Hz), 8.50 (1H, d, J=1.5 Hz), 8.92 (1H, br s), 10.13 (1H, br s).

### REFERENCE EXAMPLE 89

3-[(3-Nitrophenyl)amino]imidazo[1,5-a]pyridine was obtained in a manner similar to Example 88.

Mass: 255 (m/z, (M+H)⁺) NMR(DMSO-d₆, δ): 6.5-6.8 (2H, m), 7.27 (1H, s), 7.4-7.6 (2H, m), 7.6-7.8 (2H, m), 8.0-8.2 (1H, m), 8.3-8.4 (1H, m), 9.40 (1H, br s).

### REFERENCE EXAMPLE 90

To a solution of 2-(aminomethyl)pyridine (216 mg) in dichloromethane (30 ml) was added 3-nitrophenyl isothiocyanate (360 mg) portionwise over period of 10 minutes under stirring at ambient temperature. After stirring for an hour, the resulting precipitates were collected by filtration and washed with dichloromethane to give N-(3-nitrophenyl)-N′-(2-pyridylmethyl)thiourea (476 mg) as crystals.

Mass: 289 (m/z, (M+H)⁺) NMR(DMSO-d₆, δ): 4.85 (2H,br s), 7.2-7.5 (2H, m), 7.60 (1H, t, J=8.1 Hz), 7.7-8.0 (3H, m), 8.56 (1H, d, J=4.6 Hz), 8.65 (1H, br s), 8.72 (1H, br s), 10.31 (1H, br s).

### REFERENCE EXAMPLE 91

To a mixture of 3-[(3-nitrophenyl)amino]imidazo[1,5-a]pyridine (366 mg), ammonium chloride (37 mg), ethyl alcohol (5 ml), tetrahydrofuran (2.5 ml) and water (2.5 ml) were added Celite (400 mg) and iron powder (235 mg) under stirring at 70° C. The stirring was continued under reflux for an hour. After cooling, the reaction mixture was diluted with ethyl acetate (20 ml), filtered through Celite and evaporated. The resultant residue was chromatographed on a silica gel eluting with a mixture of dichloromethane and methanol to give N¹-(imidazo[1,5-a]pyridin-3-yl)-1,3-benzenediamine (129 mg) as crystals.

Mass: 225 (m/z, (M+H)⁺) NMR(DMSO-d₆, δ): 4.91 (2H, br s), 5.9-6.1 (1H, m), 6.1-6.4 (2H, m), 6.4-6.7 (2H, m), 6.82 (1H, t, J=7.9 Hz), 7.18 (1H, s), 7.3-7.5 (1H, m), 7.87 (1H, d, J=6.9 Hz), 8.30 (1H, br s).

### REFERENCE EXAMPLE 92

A solution of 2-amino-3-(2-thieny)benzoic acid (780 mg) in formamide (7.0 ml) was stirred under nitrogen atmosphere for 5 hours at 150° C. The mixture was poured into ice-water (1:1, 60 ml). The precipitated solid was collected by filtration, washed with water and dried to give 8-(2-thienyl)-4-quinazolinol (678 mg).

APCI-Mass: 227 (m/z, (M−H)⁺) NMR(DMSO-d₆, δ): 7.16(1H, dd, J=5.1, 3.7 Hz), 7.55(1H, t, J=7.8 Hz), 7.65(1H, dd, J=5.1, 1.1 Hz), 7.83(1H, dd, J=3.7, 1.1 Hz), 8.07(1H, d, J=7.8 Hz), 8.22(1H, s), 8.25(1H, d, J=7.8 Hz).

### REFERENCE EXAMPLE 93

To a suspension of 4-(4-fluorobenzyl)-1(2H)-phthalazinone (2.0 g) in toluene (40 ml) was added phosphorous oxychloride (4.6 ml) dropwise under nitrogen atmosphere at room temperature. The mixture was refluxed for 3.0 hours and evaporated under reduced pressure. The residue was diluted with dichloromethane and washed with water, an aqueous saturated solution of sodium hydrogencarbonate and brine. Then organic phase was dried over sodium sulfate and evaporated under reduced pressure. The crude solid was triturated with diisopropyl ether to give 1-chloro-4-(4-fluorobenzyl)phthalazine (1.89 g, 88.1%).

APCI-mass: 273 (m/z, (M+H)⁺) NMR(DMSO-d₆, δ): 4.71(2H, s), 7.10 (2H, dd, J=9.0, 6.5 Hz), 7.36 (1H, d, J=9.0 Hz), 7.40 (1H, d, J=9.0 Hz), 8.07-8.17 (2H, m), 8.29-8.42 (2H, m).

### REFERENCE EXAMPLE 94

A mixture of 4-benzylisoquinolin-2-ol (310 mg) and phosphorous oxychloride (0.775 ml) was stirred under nitrogen atmosphere for an hour at 100° C., then poured into ice-water. The mixture was diluted with ethyl acetate and washed with an aqueous saturated solution of potassium carbonate and brine. The organic layer was then dried over magnesium sulfate and evaporated under reduced pressure to give 4-benzyl-1-chloroisoquinoline (334 mg, 100%).

APCI-mass: 254 (m/z, (M+H)⁺) NMR(DMSO-d₆, δ): 4.52(2H, s), 7.14-7.28(5H, m), 7.76-7.91(2H, m), 8.13(1H, d, J=8.5 Hz), 8.28(1H, s), 8.30(1H, d, J=8.5 Hz).

### REFERENCE EXAMPLE 95

To a solution of 4-benzylisoquinoline (300 mg) in dichloromethane (3.5 ml) was added 3-chloroperoxybenzoic acid, and the mixture was stirred for 3 hours . The reaction mixture was diluted with dichloromethane and washed with water. To the solution was added potassium carbonate (4.0 g) and the resulting mixture was stirred for an hour. The mixture was then filtered off and the filtrate was evaporated under reduced pressure to give 4-benzylisoquinolin-2-ol (312 mg, 96.9%).

APCI-mass: 236 (m/z, (M+H)⁺) NMR(DMSO-d₆, δ): 4.37(2H, s), 7.19-7.32(5H, m), 7.54-7.68(2H, m), 7.89(1H, d, J=7.2 Hz), 8.02(1H, d, J=7.2 Hz), 8.09(1H, s), 8.88(1H, s).

### REFERENCE EXAMPLE 96

To a solution of isoquinoline (1.82 ml) in tetrahydrofuran (30 ml) was added a 1.0M solution of sodium triethylborohydride in tetrahydrofuran (15.5 ml) dropwise under nitrogen atmosphere at room temperature. After the mixture was stirred for 30 minutes, 2-thiophene carboxaldehyde (1.59 ml) was added to the reaction mixture in one portion via syringe. The mixture was stirred for 2 hours at room temperature and cooled to 0° C. A 0.5N aqueous solution of sodium hydroxide (30 ml) and then a 30 wt % aqueous solution of hydrogen peroxide (15 ml) were added to the reaction mixture, and the ice bath was removed. After stirring for 3 hours, the mixture was poured into water and extracted with ethyl acetate (120 ml×3). The combined extracts were washed with brine, dried over sodium sulfate and evaporated under reduced pressure to give 4-(2-thienylmethyl)isoquinoline (3.03 g, 76.7%).

APCI-mass: 226 (m/z, (M+H)⁺) NMR(DMSO-d₆, δ): 4.60(2H, s), 6.89-6.79(2H, m), 7.28-7.31(1H, m), 7.68(1H, t, J=7.0 Hz), 7.79(1H, t, J=7.0 Hz), 8.12(1H, d, J=7.0 Hz), 8.15(H, d, J=7.0 Hz), 8.79(1H, s), 9.24(1H, s).

### REFERENCE EXAMPLE 97

A mixture of 4-(2-thienylmethyl)isoquinolin-2-ol (500 mg) and phosphorous oxychloride (1.25 ml) was stirred under nitrogen atmosphere for an hour at 100° C., then poured into ice-water. The mixture was diluted with ethyl acetate and washed with an aqueous saturated solution of potassium carbonate and brine. The organic layer was then dried over magnesium sulfate and evaporated under reduced pressure. The residue was purified by a silica gel column chromatography eluting with 25% ethyl acetate in n-hexane to give 1-chloro-4-(2-thienylmethyl)isoquinoline (287 mg, 53.3%).

APCI-mass: 260 (m/z, (M+H)⁺) NMR(DMSO-d₆, δ): 4.62(2H, s), 6.90-6.94(2H, m), 7.32(1H, dd, J=5.0, 1.5 Hz), 7.73-7.95(2H, m), 8.20(1H, dd, J=7.5, 1.0 Hz), 8.30(1H, s), 8.32(1H, dd, J=7.5, 1.0 Hz).

### REFERENCE EXAMPLE 98

To a mixture of 7-iodo-1H-indole-2,3-dione (1.62 g), 3-thiopheneboronic acid (911 mg) and tetrakis(triphenylphosphine)palladium (343 mg) in 1,2-dimethoxyethane (17.5 ml) was added a solution of sodium hydrogencarbonate (997 mg) in water (17.5 ml). The mixture was refluxed for 5.0 hours, and the organic solvent was removed under reduced pressure. The residue, partially soluble in water, was extracted with ethyl acetate (150 ml×2). The combined extracts were washed with brine, dried over magnesium sulfate and evaporated under reduced pressure. The residue was purified by a silica gel column chromatography eluting with 30% ethyl acetate in n-hexane to give 7-(3-thienyl)-1H-indole-2,3-dione (779 mg, 57.2%).

APCI-mass: 228 (m/z, (M−H)⁺) NMR(DMSO-d₆, δ): 7.14(1H, t, J=7.5 Hz), 7.35(1H, dd, J=4.8, 1.4 Hz), 7.50(1H, dd, J=7.5, 1.4 Hz), 7.67-7.74(3H, m), 10.87(1H, s).

### REFERENCE EXAMPLE 99

To a stirred suspension of 7-(3-thienyl)-1H-indole-2,3-dione in a 5.0% aqueous solution of sodium hydroxide (11 ml) was added a 30% aqueous solution of hydrogen peroxide (11 ml) dropwise. The mixture was stirred for 20 minutes at 50° C. and cooled to room temperature. The filtrate was acidified to pH 3 with 1N-hydrochloric acid (5 ml), and the precipitated solid was collected by filtration, washed with water, and dried to give 2-amino-3-(3-thienyl)benzoic acid (371 mg, 77.6%).

APCI-mass: 218 (m/z, (M−H)⁺) NMR(DMSO-d₆, δ): 6.61(1H, t, J=7.4 Hz), 7.24-7.29(2H, m), 7.60-7.78(3H, m).

### REFERENCE EXAMPLE 100

A solution of 2-amino-3-(3-thieny)benzoic acid (148 mg) in formamide (1.5 ml) was stirred under nitrogen atmosphere for 6 hours at 150° C. The mixture was poured into ice-water (1:1, 20 ml). The precipitated solid was collected by filtration, washed with water and dried to give 8-(3-thienyl)-4-quinazolinol (126 mg, 81.8%).

APCI-mass: 229 (m/z, (M+H)⁺) NMR(DMSO-d₆, δ): 7.51-7.65(3H, m), 7.99-8.12(3H, m), 8.14(1H, s).

### REFERENCE EXAMPLE 101

To a mixture of 8-(3-thienyl)-4-quinazolinol (330 mg) and phosphorous oxychloride (2.7 ml) was added a small amount of N,N-dimethylformamide. The mixture was refluxed under nitrogen atmosphere and evaporated under reduced pressure. To the residue was added water, and the precipitated solid was collected by filtration. The crude solid was purified by a silica gel column chromatography eluting with 20% ethyl acetate in n-hexane to give 4-chloro-8-(3-thienyl)quinazoline (220 mg, 61.7%).

APCI-mass: 247 (m/z, (M+H)⁺) NMR(DMSO-d₆, δ): 7.70-7.76(2H, m), 7.93(1H, t, J=7.4 Hz), 8.21-8.37(3H, m), 9.16(1H, s).

### REFERENCE EXAMPLE 102

To a mixture of 3-(1,2-dimethyl-1H-imidazol-5-yl)-5-nitrophenyl methyl ether (208 mg), activated carbon(312 mg), and tetrahydrofuran(3.1 ml) were added Iron(III) chloride hexahydrate (21 mg) and hydrazine monohydrate (0.31 ml). The mixture was heated at 80 C. for 1 hour. After cooling, the reaction mixture was evaporated. The resultant was diluted with ethyl acetate(40 ml) and washed with water(30 ml×2) and brine (20 ml). The organic layer was dried over magnesium sulfate and filtered. The solvent was evaporated to give 3-(1,2-dimethyl-1H-imidazol-5-yl)-5-methoxyaniline (186 mg) as crystals.

Mass: 218 (m/z, (M+H)⁺) NMR(DMSO-d₆, δ): 2.32(3H,s), 3.49(3H, s), 3.68(3H,s), 5.20(2H, br s), 6.0-6.3(3H, m), 6.74(1H, s).

### REFERENCE EXAMPLE 103

This was prepared in a manner similar to Reference Example 102 to give 3-(1,2-dimethyl-1H-imidazol-5-yl)-5-fluoroaniline (250 mg).

Mass: 206 (m/z, (M+H)⁺) NMR(DMSO-d₆, δ): 2.32 (3H, s), 3.51 (3H, s), 5.53 (2H, br s), 6.2-6.5 (3H, m), 6.80 (1H, s).

### REFERENCE EXAMPLE 104

To a suspension of sodium hydride (1.70 g, 60% in oil) in dimethyl carbonate (36 ml) was added 7-fluoro-3,4-dihydro-1(2H)-naphtalenone (2.32 g) at ambient temperature under stirring. The mixture was heated at reflux for 3 hours. The reaction mixture was quenched with water under cooling, poured into 1N-hydrochloric acid (150 ml) and extracted with ethyl acetate (100 ml×2). The combined extracts were washed with brine (50 ml), dried over magnesium sulfate, decolorized by activated carbon, and then filtered through Celite. The filtrate was evaporated to give methyl 7-fluoro-1-oxo-1,2,3,4-tetrahydro-2-naphthalenecarboxylate (2.97 g) as yellow crystals.

Mass: 245 (m/z, (M+Na)⁺) NMR(DMSO-d₆, δ): (keto form: enol form=6:4) keto form: 2.1-2.4(2H, m), 2.9-3.1(2H, m), 3.69(3H, s), 3.88 (1H, dd, J=5.5, 10.2 Hz), 7.2-7.6 (3H, m). enol form: 2.4-2.6 (2H, m), 2.7-2.9 (2H, m), 3.80 (3H, s), 7.2-7.6 (3H, m), 12.30(1H, s).

### REFERENCE EXAMPLE 105

A mixture of methyl 7-fluoro-1-oxo-1,2,3,4-tetrahydro-2-naphtalenecarboxylate (2.90 g) and formamidine acetate (5.43 g) was heated with stirring for an hour at 180° C. After cooling, the reaction mixture was partitioned between 1N-aqueous solution of sodium hydroxide (200 ml) and dichloromethane (100 ml). The organic layer was extracted with 1N-aqueous solution of sodium hydroxide (100 ml) again, and the combined aqueous layers were washed with dichloromethane (100 ml×2) and neutralized with conc. hydrochloric acid. Resultant precipitates were collected by filtration, washed with water (50 ml×3) and dried under reduced pressure for 5 hours at 50° C. to give 9-fluoro-5,6-dihydrobenzo[h]quinazolin-4-ol (1.80 g) as a pale brown solid.

Mass: 217 (m/z, (M+H)⁺) NMR(DMSO-d₆, δ): 2.6-3.0(4H, m), 7.1-7.4 (2H, m), 7.74 (1H, dd, J=2.8, 10.0 Hz), 8.19 (1H, s), 12.54 (1H, br s).

### REFERENCE EXAMPLE 106

To a mixture of 1H-imidazol-4-ylmethanol hydrochloride (4.55 g), imidazole (11.5 g) in N,N-dimethylformamide (46 ml) was added tert-butyldimethylsilyl chloride (15.3 g) at 0° C. After stirring for 14 hours at ambient temperature, the reaction mixture was poured into water (heat evolution) and extracted with ethyl acetate (200 ml×2). The combined organic extracts were washed with an aqueous saturated solution of sodium hydrogencarbonate (200 ml), water (200 ml×2) and brine (200 ml). The organic layer was dried over magnesium sulfate and filtered. After evaporation of the solvent, the residue was chromatographed on silica gel eluting with a mixture of dichloromethane-methanol (1%, 2% and then 4%) to give tert-butyl(dimethyl)silyl 1H-imidazol-4-ylmethyl ether (6.68 g) as colorless crystals.

Mass: 213 (m/z, (M+H)⁺) NMR(DMSO-d₆, δ): 0.03(6H,s), 0.85(9H, s), 4.53(2H,s), 6.87(1H, br s), 7.51(1H, br s), 11.88(1H, br s).

### REFERENCE EXAMPLE 107

In a 500 ml flask equipped with a magnetic stirrer bar were charged tert-butyl(dimethyl)silyl 1H-imidazol-4-ylmethyl ether (1.20 g), 3-nitrophenylboronic acid (1.13 g), anhydrous cupric acetate (1.54 g), pyridine (0.67 g), molecular sieves 3A (5.0 g) and dichloromethane (48 ml). The mixture was stirred under air for 12 hours at ambient temperature. After concentration of the reaction mixture under reduced pressure, the residue was chromatographed on silica gel eluting with a mixture of dichloromethane and methanol (1%) to give 4-({[tert-butyl(dimethyl)silyl]oxy}methyl)-1-(3-nitrophenyl)-1H-imidazole (185 mg) as crystals.

Mass: 334 (m/z, (M+H)⁺) NMR(DMSO-d₆, δ): 0.10(6H,s), 0.90(9H, s), 4.61(2H,s), 7.7-7.9(2H, m), 8.1-8.3(2H, m), 8.41(1H, br s), 8.48 (1H, t, J=2.1 Hz).

### REFERENCE EXAMPLE 108

To a solution of 4-({[tert-butyl(dimethyl)silyl]oxy}methyl)-1-(3-nitrophenyl)-1H-imidazole (227 mg) in methanol (5 ml) was added 10% palladium on carbon (50% wet, 40 mg). The resultant mixture was hydrogenated under atmospheric pressure of hydrogen gas for 12 hours. The catalyst was removed by filtration and the filtrate was concentrated under reduced pressure to give 3-[4-({[tert-butyl(dimethyl)silyl]oxy}methyl)-1H-imidazol-1-yl]aniline (204 mg) as a pale yellow oil.

Mass: 304 (m/z, (M+H)⁺) NMR(DMSO-d₆, δ): 0.08(6H,s), 0.89(9H, s), 4.58 (2H, s), 5.38 (2H, br s), 6.4-6.8 (3H, m), 7.0-7.2 (1H, m), 7.37 (1H, br s), 7.99 (1H, d, J=1.4 Hz).

### EXAMPLE 1

To a suspension of (3-aminophenyl)-(5-phenylisoquinolin-1-yl)amine (0.1 g) in ethanol (5 ml) was added methyl thiobenzimidate hydroiodide (90 mg), and the mixture was heated to 90° C. for 2 hours. After cooling to ambient temperature, the reaction mixture was taken up into a mixture of dichloromethane and an aqueous potassium carbonate solution (10%). The separated organic layer was washed with brine, dried over potassium carbonate, and evaporated under reduced pressure. The residue was purified by a column chromatography on silica gel eluting with 0-20% methanol in dichloromethane to give N-[3-(5-phenylisoquinoline-1-ylamino)phenyl]benzamidine (52 mg).

APCI-mass; 415 (m/z, [M+H]⁺) NMR (DMSO-d₆, δ): 6.65 (1H, d, J=7.3 Hz), 7.00 (1H, d, J=6.1 Hz), 7.21-7.42 (1H, m), 7.42-7.76 (14H, m), 7.85-8.05 (3H, m), 8.59 (1H, d, J=7.0 Hz), 9.26 (1H, s).

### EXAMPLE 2

4-Fluoro-N-[3-(5-phenylisoquinolin-1-ylamino)phenyl]benzamidine (61 mg) was obtained from (3-aminophenyl)-(5-phenylisoquinolin-1-yl)amine (0.1 g) and 4-fluoro-thiobenzimidic acid methyl ester hydroiodide (105 mg) in a manner similar to Example 1.

APCI-mass; 433.2 (m/z, [M+H]⁺), NMR (DMSO-d₆, δ): 6.60 (1H, d, J=7.3 Hz), 7.00 (1H, d, J=6.1 Hz), 7.21-7.42 (3H, m), 7.42-7.80 (11H, m), 7.85-8.15 (3H, m), 8.59 (1H, d, J=7.0 Hz), 9.23 (1H, s).

### EXAMPLE 3

[6-(2-Methylpyridin-3-yloxy)-pyridin-3-yl]-(5-phenyl-isoquinolin-1-yl)amine (109 mg) was obtained from 1-chloro-5-phenylisoquinoline (78 mg) and [6-(2-methylpyridin-3-yloxy)pyridin-3-yl]amine (131 mg) in a manner similar to Reference Example 4 FR235762.

APCI-mass; 405 (m/z, [M+H]⁺) NMR (DMSO-d₆, δ): 2.35 (3H, s), 7.15 (1H, d, J=8.9 Hz), 7.30 (1H, dd, J=4.7, 8.0 Hz), 7.38-7.60 (6H, m), 7.60-7.84 (3H, m), 7.90 (1H, s), 8.28-8.40 (2H, m), 8.52-8.67 (2H, m), 9.41 (1H, s).

### EXAMPLE 4

A mixture of 1-chloro-5-phenylisoquinoline (0.162 g) and 3-(imidazol-1-yl)aniline (215 mg) was heated to 190° C. for 5 minutes. After cooling to ambient temperature, the reaction mixture was taken up into a mixture of ethyl acetate and an aqueous potassium carbonate solution (10%). The separated organic layer was washed with brine, dried over potassium carbonate and evaporated under reduced pressure. The residue was purified by a column chromatography on silica gel (60 ml) eluting with 0-4% methanol in dichloromethane to give (3-imidazol-1ylphenyl)-(5-phenylisoquinolin-1-yl)amine (100 mg).

APCI-mass; 363 (m/z, [M+H]⁺) NMR (DMSO-d₆, δ): 7.07 (1H, d, J=6.0 Hz), 7.13 (1H, s), 7.24 (1H, d, J=7.1 Hz), 7.40-7.85 (9H, m), 7.90 (1H, d, J=7.5 Hz), 8.03 (1H, d, J=6.0 Hz) 8.20-8.28 (2H, m), 8.59 (1H, d, J=8.1 Hz), 9.47 (1H, s).

### EXAMPLE 5

To a solution of (3-aminomethylphenyl)-(5-phenylisoquinolin-1-yl)amine (50 mg) in dimethoxyethane (1 ml) were added 2-chloro-1H-benzoimidazole (46 mg) and N,N-diisopropylethylamine (0.13 ml), and the mixture was heated to 130° C. for 60 hours. After cooling to ambient temperature, the reaction mixture was taken up into a mixture of an aqueous potassium carbonate solution (10%) and ethyl acetate. The separated organic layer was washed with brine, dried over potassium carbonate and evaporated to dryness. The residue was purified by a column chromatography on silica gel (60 ml) eluting with 0-6% methanol in dichloromethane. The obtained product was triturated with diisopropyl ether to give {3-[(1H-benzoimidazol-2-ylamino)methyl]phenyl}-(5-phenyl-isoquinolin-1-yl) amine (32 mg).

APCI-mass; 442 (m/z, [M+H]⁺), NMR (DMSO-d₆, δ): 4.55 (2H, d, J=5.9 Hz), 6.87-6.94 (2H, m), 6.96-7.90 (17H, m), 8.62 (1H, d, J=8.8 Hz), 9.28 (1H, s).

### EXAMPLE 6

To a solution of (3-aminomethylphenyl)-(5-phenylisoquinolin-1-yl)amine (50 mg) in dimethoxyethane (1 ml) were added 2-chloro-1-methyl-1H-benzoimidazole (87 mg) and N,N-diisopropylethylamine (0.13 ml), and the mixture was heated to 130° C. for 48 hours. After cooling o ambient temperature, the reaction mixture was taken up into a mixture of an aqueous potassium carbonate solution (10%) and ethyl acetate. The separated organic layer was washed with brine, dried over potassium carbonate and evaporated. The residue was purified by a column chromatography on silica gel (60 ml) eluting with 0-6% methanol in dichloromethane. The obtained product was triturated with diisopropyl ether to give {3-[(1-methyl-1H-benzoimidazol-2-ylamino)methyl]phenyl}-(5-phenylisoquinolin-1-yl)amine (28 mg).

APCI-mass; 456(m/z, [M+H]⁺), NMR(DMSO-d₆, δ); 3.56 (3H, s), 4.65 (2H, d, J=5.9 Hz), 6.85-7.95 (18H, m), 8.59 (1H, d, J=8.9 Hz), 9.27 (1H, s).

### EXAMPLE 7

A mixture of 1-chloro-5-(thiophen-3-yl)isoquinoline (0.1 g) and 3-(imidazol-1-yl)aniline (194 mg) was heated to 190° C. for 30 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0-4% methanol in dichloromethane to give [3-(imidazol-1-yl)phenyl]-[5-(thiophen-3-yl)isoquinolin-1-yl]amine (3.3 mg).

APCI-mass; 369 (m/z, [M+H]⁺) NMR (DMSO-d₆, δ): 7.05-8.10 (12H, m), 8.10-8.30 (2H, m), 8.55 (1H, d, J=8.1 Hz), 9.45 (1H, s).

### EXAMPLE 8

A mixture of 1-chloro-5-(thiophen-3-yl)isoquinoline (65 mg) and 3-(pyrimidin-5-yl)phenylamine (90 mg) was heated to 190° C. for 10 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0-2% methanol in dichloromethane to give [3-(pyrimidin-5-yl)phenyl]-[5-(thiophen-3-yl)isoquinolin-1-yl]amine (42 mg).

APCI-mass; 381 (m/z, [M+H]⁺) NMR (DMSO-d₆, δ): 7.23 (1H, d, J=6.2 Hz), 7.30-7.82 (7H, m), 7.95-8.10 (2H, m), 8.27 (1H, s), 8.57 (1H, d, J=7.7 Hz), 9.14 (2H, s), 9.21 (1H, s), 9.43 (1H, s).

### EXAMPLE 9

A mixture of 1-chloro-5-(thiophen-3-yl)isoquinoline (73 mg) and 3-([1,2,4]triazol-1-yl)phenylamine (95 mg) was heated to 190° C. for 10 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0%-2% methanol in dichloromethane to give [5-(thiophen-3-yl)isoquinolin-1yl]-[3-([1,2,4]triazol-1-yl)phenyl]amine (52 mg).

APCI-mass; 370 (m/z, [M+H]⁺) NMR (DMSO-d₆, δ): 7.25 (1H, d, J=6.0 Hz), 7.37 (1H, d, J=4.7 Hz), 7.42-7.60 (2H, m), 7.60-7.84 (4H, m), 7.90-8.00 (1H, m), 8.06 (1H, d, J=6.0 Hz), 8.26 (1H, s), 8.50 (1H, s), 8.58 (1H, d, J=7.8 Hz), 9.27 (1H, s), 9.52 (1H, s).

### EXAMPLE 10

A mixture of 1-chloro-5-(thiophen-3-yl)isoquinoline (66 mg) and 3-(2,3-dimethyl-3H-imidazol-4-yl)-phenylamine (100 mg) was heated to 190° C. for 10 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0%-2% methanol in dichloromethane to give ([3-(2,3-dimethyl-3H-imidazol-4-yl)phenyl]-[5-(thiophen-3-yl)isoquinolin-1-yl]amine (32 mg).

APCI-mass; 397 (m/z, [M+H]⁺) NMR (DMSO-d₆, δ): 2.37 (3H, s), 3.60 (3H, s), 6.90 (1H, s), 7.04 (1H, d, J=7.8 Hz), 7.20 (1H, d, J=5.9 Hz), 7.30-7.50 (2H, m), 7.57-7.80 (4H, m), 7.87 (1H, d, J=8.8 Hz), 7.92-8.08 (2H, m), 8.55 (1H, d, J=8.2 Hz), 9.34 (1H, s).

### EXAMPLE 11

A mixture of 1-chloro-5-(thiophen-3-yl)isoquinoline (80 mg) and [6-(2-methylpyridin-3-yloxy)pyridin-3-yl]amine (131 mg) was heated to 190° C. for 10 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a gel permeation chromatography (JAIGEI-1H/2H) eluting with 0.5% triethylamine in chloroform to give [6-(2-methylpyridin-3-yloxy)pyridin-3-yl]-(5-thiophen-3-ylisoquinolin-1-yl)amine (81 mg).

APCI-mass; 411 (m/z, [M+H]⁺) NMR (DMSO-d₆, δ): 2.35 (3H, s), 7.13 (1H, d, J=8.8 Hz), 7.19 (1H, d, J=6.0 Hz), 7.24-7.42 (2H, m), 7.51 (1H, d, J=8.1 Hz), 7.60-7.82 (4H, m), 7.95 (1H, d, J=6.0 Hz), 8.30-8.40 (2H, m), 8.43-8.60 (2H, m), 9.36 (1H, s).

### EXAMPLE 12

A mixture of 1-chloro-5-(thiophen-3-yl)isoquinoline (90 mg) and 4-methyl-3-(pyrimidin-5-yl)phenylamine (136 mg) was heated to 190° C. for 10 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a gel permeation chromatography (JAIGEI-1H/2H) eluting with 0.5% triethylamine in chloroform to give [4-methyl-3-(pyrimidin-5-yl)phenyl]-[5-(thiophen-3-yl)isoquinolin-1-yl]amine (81 mg).

APCI-mass; 395 (m/z, [M+H]⁺) NMR (DMSO-d₆, δ): 2.25 (3H, s), 7.18 (1H, d, J=6.0 Hz), 7.26-7.40 (2H, m), 7.60-7.80 (4H, m), 7.83 (1H, d, J=2.2 Hz), 7.90-8.08 (2H, m), 8.54 (1H, d, J=7.3 Hz), 8.91 (2H, s), 9.23 (1H, s), 9.29 (1H, s).

### EXAMPLE 13

A mixture of 1-chloro-5-bromoisoquinoline (65 mg) and 3-(imidazol-1-yl)aniline (86 mg) was heated to 190° C. for 5 minutes. After cooling to ambient temperature, the reaction mixture was taken up into a mixture of ethyl acetate and an aqueous potassium carbonate solution (10%). The separated organic layer was washed with brine, dried over potassium carbonate and evaporated under reduced pressure. The residue was purified by a column chromatography on silica gel (50 ml) eluting with 0-3% methanol in dichloromethane to give (5-bromoisoquinolin-1-yl)-[3-(imidazol-1-yl)phenyl]amine (54 mg).

APCI-mass; 365, 367 (m/z, [M+H]⁺) NMR (DMSO-d₆, δ): 7.13 (1H, s), 7.23-7.67(4H, m), 7.68 (1H, s), 7.85 (1H, d, J=9.1 Hz), 8.08-8.28 (4H, m), 8.60 (1H, d, J=8.3 Hz), 9.52 (1H, s).

### EXAMPLE 14

A mixture of 1-chloro-5-bromoisoquinoline (52 mg) and 3-(2,3-dimethyl-3H-imidazol-4-yl)aniline (80 mg) was heated to 190° C. for an hour. After cooling to ambient temperature, the reaction mixture was taken up into a mixture of ethyl acetate and an aqueous potassium carbonate solution (10%). The separated organic layer was washed with brine, dried over potassium carbonate and evaporated under reduced pressure. The residue was purified by a column chromatography on silica gel (50 ml) eluting with 0-3% methanol in dichloromethane to give (5-bromoisoquinolin-1-yl)-[3-(2,3-dimethyl-3H-imidazol-4-yl)phenyl]amine (23 mg).

APCI-mass; 393, 395 (m/z, [M+H]⁺) NMR (DMSO-d₆, δ): 2.37 (3H, s), 3.59 (3H, s), 6.87 (1H, s), 7.07 (1H, d, J=7.6 Hz), 7.30-7.47 (2H, m), 7.56 (1H, t, J=7.8 Hz), 7.83 (1H, d, J=7.6 Hz), 7.94 (1H, s), 8.03-8.23 (2H, m), 8.59 (1H, d, J=8.5 Hz), 9.42 (1H, s).

### EXAMPLE 15

To a solution of (5-bromoisoquinolin-1-yl)-[3-(2,3-dimethyl-3H-imidazol-4-yl)phenyl]amine (0.1 g) in a mixture of dimethoxyethane (0.8 ml) and an aqueous sodium carbonate solution (2 M, 0.4 ml) were added 4-fluorophenylboronic acid (46 mg) and tetrakis(triphenylphosphine)-palladium (0) (14 mg) under nitrogen, and the mixture was heated to 100° C. for 3 hours. After cooling to ambient temperature, the separated organic layer was evaporated under reduced pressure. The residue was taken up into ethyl acetate. The mixture was washed in turn with an aqueous potassium carbonate solution (10%) and brine, dried over sodium sulfate and evaporated. The residue was purified by a gel permeation chromatography (JAIGEI-1H/2H) eluting with 0.5% triethylamine in chloroform to give [3-(2,3-dimethyl-3H-imidazol-4-yl)phenyl]-[5-(4-fluorophenyl)isoquinolin-1-yl]amine (1.22 g).

APCI-mass; 409 (m/z, [M+H]⁺) NMR (DMSO-d₆, δ): 2.37 (3H, s), 3.60 (3H, s), 6.87 (1H, s), 6.99 (1H, d, J=6.0 Hz), 7.04 (1H, d, J=7.8 Hz), 7.29-7.46 (3H, m), 7.46-7.60 (2H, m), 7.60-7.80 (2H, m), 7.87 (1H, d, J=8.1 Hz), 7.92-8.04 (2H, m), 8.58 (1H, d, J=7.1 Hz), 9.35 (1H, s).

### EXAMPLE 16

A mixture of 1-chloro-5-bromoisoquinoline (71 mg) and 3-(pyrimidin-5-yl)-phenylamine (100 mg) was heated to 190° C. for 10 minutes. After cooling to ambient temperature, the reaction mixture was taken up into a mixture of ethyl acetate and an aqueous potassium carbonate solution (10%). The separated organic layer was washed with brine, dried over potassium carbonate and evaporated under reduced pressure. The residue was purified by a column chromatography on silica gel (60 ml) eluting with 0-3% methanol in dichloromethane to give (5-bromoisoquinolin-1-yl)-[3-(pyrimidin-5-yl)phenyl]amine (42 mg).

APCI-mass; 377, 379 (m/z, [M+H]⁺) NMR (DMSO-d₆, δ): 7.36 (1H, d, J=6.1 Hz), 7.40-7.70 (3H, m), 7.94-8.31 (4H, m), 8.61 (1H, d, J=8.4 Hz), 9.14 (2H, s), 9.22 (1H, s), 9.49 (1H, s).

### EXAMPLE 17

To a suspension of (3-aminophenyl)-(5-bromoisoquinolin-1-yl)amine (80 mg) in ethanol (2 ml) was added methyl thiobenzimidate hydroiodide (86 mg), and the mixture was heated to 90° C. for 2 hours. After cooling to ambient temperature, the reaction mixture was taken up into a mixture of dichloromethane and an aqueous potassium carbonate solution (10%). The separated organic layer was washed with brine, dried over potassium carbonate, and evaporated under reduced pressure. The residue was purified by a column chromatography on silica gel eluting with 0-20% methanol in dichloromethane gave N-[3-(5-bromoisoquinolin-1-ylamino)phenyl]benzamidine (52 mg).

APCI-mass; 415, 417 (m/z, [M+H]⁺) NMR (DMSO-d₆, δ): 6.50-6.80 (3H, m), 7.20-7.40 (2H, m), 7.40-7.70 (6H, m), 7.88-8.22 (4H, m), 8.63 (1H, d, J=8.3 Hz), 9.29 (1H, s).

### EXAMPLE 18

A mixture of 1-chloro-5-bromoisoquinoline (68 mg) and [6-(2-methylpyridin-3-yloxy)pyridin-3-yl]amine (112 mg) was heated to 190° C. for 7minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0-5% methanol in dichloromethane to give (5-bromo-isoquinolin-1-yl)-[6-(2-methylpyridine-3-yloxy)pyridin-3-yl]amine (30 mg).

APCI-mass; 407, 409 (m/z, [M+H]⁺) NMR (DMSO-d₆, δ): 2.34 (3H, s), 7.14 (1H, d, J=8.9 Hz), 7.28-7.40 (2H, m), 7.45-7.65 (2H, m), 6.02-8.17 (2H, m), 8.25-8.37 (2H, m), 8.48 (1H, d, J=2.5 Hz), 8.54 (1H, d, J=8.3 Hz), 9.45 (1H, s).

### EXAMPLE 19

A mixture of 1-chloroisoquinoline (41 mg) and [6-(2-methylpyridin-3-yloxy)pyridin-3-yl]amine (100 mg) was heated to 190° C. for 10 minutes. After cooling to ambient temperature, the reaction mixture was taken up into a mixture of ethyl acetate and an aqueous potassium carbonate solution (10%). The separated organic layer was washed with brine, dried over potassium carbonate and evaporated. The residue was purified by a column chromatography on silica gel (60 ml) eluting with 0-3% methanol in dichloromethane to give (isoquinolin-1-yl)-[6-(2-methylpyridin-3-yloxy)pyridin-3-yl]amine (32 mg).

APCI-mass; 329 (m/z, [M+H]⁺) NMR (DMSO-d₆, δ): 2.36 (3H, s), 7.13 (1H, d, J=8.8 Hz), 7.19 (1H, d, J=5.8 Hz), 7.30(1H, dd, J=4.6, 8.1 Hz), 7.50 (1H, d, J=8.0 Hz), 7.55-7.86 (3H, m), 7.96 (1H, d, J=5.7 Hz), 8.30-8.42 (2H, m), 8.42-8.56 (2H, m), 9.30 (1H, s)

### EXAMPLE 20

To a suspension of (3-aminophenyl)-(isoquinolin-1-yl)amine (0.1 g) in ethanol (3 ml) was added methyl thiobenzimidate hydroiodide (120 mg), and the mixture was heated to 90° C. for 2 hours. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chlomatography on silica gel eluting with 0-20% methanol in dichloromethane to give N-[3-(isoquinolin-1-ylamino)phenyl]benzamidine (68 mg).

APCI-mass; 339 (m/z, [M+H]⁺) NMR (DMSO-d₆, δ): 6.27 (2H, brs), 6.50 (1H, d, J=7.9 Hz), 7.16 (1H, d, J=5.7 Hz), 7.25 (1H, t, J=7.9 Hz), 7.40-7.85 (8H, m), 7.95-8.08 (3H, m), 8.54 (1H, d, J=8.1 Hz), 9.08 (1H, s)

### EXAMPLE 21

A mixture of 1-chloro-4-phenylisoquinoline (0.20 g) and 3-imidazol-1-ylphenylamine (0.132 g) was heated to 200° C. for 5 minutes. After cooling to ambient temperature, the reaction mixture was taken up into a mixture of dichloromethane and an aqueous potassium carbonate solution (10%). The separated organic layer was washed with brine, dried over potassium carbonate and evaporated. The residue was purified by a column chromatography on silica gel (60 ml) eluting with 0-20% methanol in dichloromethane. The obtained product was triturated with diisopropyl ether to give [3-(imidazol-1-yl)phenyl]-(4-phenylisoquinolin-1-yl)amine (34 mg).

APCI-mass; 363 (m/z, [M+H]⁺) NMR (DMSO-d₆, δ): 7.13 (1H, s), 7.24 (1H, d, J=9.2 Hz), 7.39-7.61 (6H, m), 7.63-7.81 (4H, m), 7.91 (1H, d, J=9.2 Hz), 8.00 (1H, s), 8.20-8.25 (2H, m), 8.55-8.67 (1H, m), 9.49 (1H, s)

### EXAMPLE 22

A mixture of 1-chloro-5-(4-fluorophenyl)isoquinoline (150 mg) and 3-(imidazol-1-yl)aniline (185 mg) was heated to 190° C. for 10 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0-2% methanol in dichloromethane to give [3-(imidazol-1-yl)phenyl]-[5-(4-fluorophenyl)isoquinolin-1-yl]amine (23 mg).

APCI-mass; 381 (m/z, [M+H]⁺) NMR (DMSO-d₆, δ): 7.03 (1H, d,J=6.0 Hz), 7.13 (1H,s), 7.24-7.35 (1H, m), 7.36-7.60 (5H, m), 7.65-7.78 (3H, m), 7.82-7.95 (1H, m), 8.03 (1H, d, J=6.0 Hz), 8.13-8.26 (2H, m), 8.59 (1H, d, J=6.8 Hz), 9.47 (1H, s).

### EXAMPLE 23

To a suspension of N-(benzo[d]isoxazol-3-yl)benzene-1,3-diamine (42 mg) in ethanol (2 ml) was added methyl thiobenzimidate hydroiodide (38.5 mg), and the mixture was heated to 90° C. for 2 hours. After cooling to ambient temperature, the reaction mixture was taken up in to a mixture of ethyl acetate and an aqueous potassium carbonate solution (10%) The separated organic layer was washed with brine, dried over potassium carbonate, and evaporated under reduced pressure. The residue was purified by a column chromatography on silica gel eluting with 0-3% methanol in dichloromethane. The obtained product was triturated with diisopropyl ether to give N-[3-(benzo[d]isoxazol-3-ylamino)phenyl]benzamidine (31 mg).

APCI-mass; 329 (m/z, [M+H]⁺) NMR (DMSO-d₆, δ): 6.30 (2H, brs), 6.48 (1H, d, J=7.0 Hz), 7.20-7.55 (7H, m), 7.55-7.72 (2H, m), 7.90-8.08 (2H, m), 8.16 (1H, d, J=7.7 Hz), 9.46 (1H, s).

### EXAMPLE 24

To a solution of N-[3-(benzo[d]isoxazol-3-ylamino)phenyl]benzamidine (1.3 g) in ethyl acetate (2 ml) was added a solution of hydrogen chloride in ethyl acetate (4 N, 3.3 ml) at ambient temperature. The resultant crystalline was collected by filtration, washed with ethyl acetate and dried in vacuo to give N-[3-(benzo[d]isoxazol-3-yl)phenyl]benzamidine hydrochloride (1.45 g).

APCI-mass; 329 (m/z, [free form of M+H]⁺) mp>250° C. NMR (DMSO-d₆, δ): 7.07 (1H, d, J=7.5 Hz), 7.32-7.50 (1H, m), 7.50-7.90 (5H, m), 7.90-8.05 (3H, m), 8.38 (1H, d, J=7.8 Hz), 10.28 (1H, s).

### EXAMPLE 25

To a solution of N-[3-(benzo[d]isoxazol-3-ylamino)phenyl]benzamidine (0.936 g) in methanol (4.6 ml) was added methanesulfonic acid (0.185 ml) at ambient temperature. To the mixture was added diisopropyl ether (20 ml). The resultant precipitate was collected by filtration, washed with diisopropyl ether and dried in vacuo to give N-[3-(benzo[d]isoxazol-3-ylamino)phenyl]benzamidine methanesulfonate (0.78 g).

APCI-mass; 329 (m/z, [free form of M+H]⁺), mp: 113-115° C. NMR (DMSO-d₆, δ): 7.07 (1H, d, J=7.5 Hz), 7.32-7.50 (1H, m), 7.50-7.90 (5H, m), 7.90-8.05 (3H, m), 8.38 (1H, d, J=7.8 Hz), 10.28 (1H, s).

### EXAMPLE 26

To a suspension of N-(benzo[d]isoxazol-3-yl)benzene-1,3-diamine (0.1 g) in ethanol (3 ml) was added thiophene-2-carboximidothioic acid methyl ester hydroiodide (0.13 g), and the mixture was heated to 90° C. for 2 hours. After cooling to ambient temperature, the reaction mixture was taken up into a mixture of ethyl acetate and an aqueous potassium carbonate solution (10%). The separated organic layer was washed with brine, dried over potassium carbonate and evaporated under reduced pressure. The residue was purified by a column chromatography on silica gel eluting with 0-3% methanol in dichloromethane. The obtained product was triturated with a mixture of dichloromethane and diisopropyl ether to give N-[3-(benzo[d]isoxazol-3-ylamino)phenyl]thiophene-2-carboxamidine (90 mg).

APCI-mass; 335 (m/z, [M+H]⁺) NMR (DMSO-d₆, δ): 6.35-6.60 (3H, m), 7.05-7.20 (1H, m), 7.20-7.50 (4H, m), 7.58-7.70 (3H, m), 7.70-7.85 (1H, m), 8.16 (1H, d, J=8.0 Hz), 9.46 (1H, s).

### EXAMPLE 27

To a suspension of N-(7-phenylbenzo[d]isoxazol-3-yl)benzene-1,3-diamine (74 mg) in ethanol (2 ml) was added methyl thiobenzimidate hydroiodide (82 mg), and the mixture was heated to 90° C. for 1.5 hours. After cooling to ambient temperature, the reaction mixture was taken up into a mixture of ethyl acetate and an aqueous potassium carbonate solution (10%). The separated organic layer was washed with brine, dried over potassium carbonate and evaporated under reduced pressure. The residue was purified by a column chromatography on silica gel (50 ml) eluting with 0-25% methanol in dichloromethane. The obtained product was triturated with diisopropyl ether to give N-[3-(7-phenylbenzo[d]isoxazol-3-ylamino)phenyl]benzamidine (45 mg).

APCI-mass; 405 (m/z, [M+H]⁺) NMR (DMSO-d₆, δ): 6.62 (1H, d, J=7.3 Hz), 6.95 (2H, brs), 7.30-7.60 (10H, n), 7.83-8.05 (5H, m), 8.18 (1H, d, J=7.9 Hz), 9.61 (1H, s).

### EXAMPLE 28

To a suspension of N-(7-bromobenzo[d]isoxazol-3-yl)benzene-1,3-diamine (50 mg) in ethanol (2 ml) was added methyl thiobenzimidate hydroiodide (46 mg), and the mixture was heated to 90° C. for an hour. After cooling to ambient temperature, the reaction mixture was take n up into a mixture of ethyl acetate and an aqueous potassium carbonate solution (10%). The separated organic layer was washed with brine, dried over potassium carbonate and evaporated under reduced pressure. The residue was purified by a column chromatography on silica gel (50 ml) eluting with 0-20% methanol in dichloromethane. The obtained product was triturated with diisopropyl ether to give N-[3-[(7-bromo-benzo[d]isoxazol-3-yl)amino]phenyl]benzamidine (45 mg).

APCI-mass; 407, 409 (m/z, [M+H]⁺) NMR (DMSO-d₆, δ): 6.63 (1H, d, J=7.0 Hz), 6.95 (2H, brs), 7.30-7.70 (7H, m), 7.85-8.12 (3H, m), 8.20 (1H, d, J=7.2 Hz), 9.68 (1H, s)

### EXAMPLE 29

To a solution of [6-(2-methylpyridin-3-yloxy)pyridin-3-yl]amine (0.22 g) in tetrahydrofurane (5 ml) was added dropwise a solution of n-butyl lithium in n-hexane (1.54 M, 0.62 ml) at 0° C. The mixture was allowed to stir at 0° C. for 30 minutes, and to the mixture was added a solution of 3-chlorobenzo[d]isoxazole (0.1 g) in tetrahydrofuran (3 ml) at 0° C. The reaction mixture was allowed to stir at ambient temperature for 15 hours, and was taken up into a mixture of ethyl acetate and an aqueous ammonium chloride solution. The separated organic layer was washed well with water, dried over potassium carbonate and evaporated under reduced pressure. The residue was purified by a column chromatography on silica gel (60 ml) eluting with 0-3% methanol in dichloromethane. The obtained product was triturated with diisopropyl ether to give (benzo[d]isoxazol-3-yl)-[6-(2-methylpyridin-3-yloxy)pyridin-3-yl]amine (49 mg).

APCI-mass; 319 (m/z, [M+H]⁺) NMR (DMSO-d₆, δ): 2.34 (3H, s), 7.18 (1H, d, J=8.8 Hz), 7.23-7.45 (2H, m), 7.49 (1H, d, J=8.0 Hz), 7.55-7.63 (2H, m), 8.10 (1H, d, J=7.9 Hz), 8.20-8.39 (2H, m), 8.42 (1H, d, J=2.8 Hz), 9.75 (1H, s).

### EXAMPLE 30

A mixture of 1-chloro-5-(pyrrol-1-yl)isoquinoline (90 mg) and 3-([1,2,4]triazol-1-yl)phenylamine (126 mg) was heated to 190° C. for 5 minutes. After cooling to ambient temperature, the reaction mixture was taken up into a mixture of ethyl acetate and an aqueous potassium carbonate solution (10%). The separated organic layer was washed with brine, dried over potassium carbonate and evaporated. The obtained residue was triturated with methanol to give [5-(pyrrol-1-yl)isoquinolin-1-yl]-[3-([1,2,4]triazol-1-yl)phenyl]amine (33 mg).

APCI-mass; 353 (m/z, [M+H]⁺) NMR (DMSO-d₆, δ): 6.25-6.4 (2H, m), 6.90 (1H, d, J=6.0 Hz), 7.05-7.20 (2H, m), 7.40-7.60 (2H, m), 7.65-7.80 (2H, m), 7.83-8.01 (1H, m), 8.08 (1H, d, J=6.0 Hz), 8.25 (1H, s), 8.45-8.55 (1H, m), 8.55-8.73 (1H, m), 9.27 (1H, s), 9.61 (1H, s).

### EXAMPLE 31

A mixture of 1-chloro-5-(pyrrol-1-yl)isoquinoline (90 mg) and 3-(2,3-dimethyl-3H-imidazol-4-yl)-phenylamine (147 mg) was heated to 190° C. for an hour. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0-8% methanol in dichloromethane to give [3-(2,3-dimethyl-3H-imidazoyl-4-yl)phenyl]-[5-(pyrrol-1-yl)isoquinolin-1-yl]amine (21 mg).

APCI-mass; 380 (m/z, [M+H]⁺) NMR (DMSO-d₆, δ): 2.37 (3H, s), 3.60 (3H, s), 6.30 (2H, t, J=2.0 Hz), 6.82-7.00 (2H, m), 7.00-7.20 (3H, m), 7.41 (1H, t, J=8.0 Hz), 7.65-7.80 (2H, m), 7.80-8.10 (3H, m), 8.55-8.65 (1H, m), 9.42 (1H, s).

### EXAMPLE 32

A mixture of 1-chloro-5-(pyrrol-1-yl)isoquinoline (90 mg) and [6-(2-methylpyridin-3-yloxy)pyridin-3-yl]amine (158 mg) was heated to 190° C. for 8 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0-3% methanol in dichloromethane to give [6-(2-methylpyridin-3-yloxy)pyridin-3-yl]-[5-(pyrrol-1-yl)isoquinolin-1-yl]amine (75 mg).

APCI-mass; 394 (m/z, [M+H]⁺) NMR (DMSO-d₆, δ): 2.35 (3H, s), 6.34 (2H, t, J=2.0 Hz), 6.85 (1H, d, J=6.0 Hz), 7.06 (2H, t, J=2.0 Hz), 7.14 (1H, d, J=8.8 Hz), 7.30 (1H, dd, J=4.7, 8.1 Hz), 7.51 (1H, d, J=8.1 Hz), 7.60-7.80 (2H, m), 7.96 (1H, d, J=6.0 Hz), 8.25-8.40 (2H, m), 8.45-8.63 (2H, m), 9.45 (1H, s).

### EXAMPLE 33

A mixture of 1-chloro-5-(pyrrol-1-yl)isoquinoline (90 mg) and 4-methyl-3-(pyrimidin-5-yl)phenylamine (145 mg) was heated to 190° C. for 8 minutes. After cooling to ambient temperature, the reaction mixture was taken up into a mixture of dichloromethane and an aqueous potassium carbonate solution (10%). The separated organic layer was washed with brine, dried over potassium carbonate and evaporated under reduced pressure. The residue was purified by a column chromatography on silica gel (60 ml) eluting with 0-2% methanol in dichloromethane to give [4-methyl-3-(pyrimidin-5-yl)phenyl]-[5-(pyrrol-1-yl)isoquinolin-1-yl]amine (60 mg).

APCI-mass; 378 (m/z, [M+H]⁺) NMR (DMSO-d₆, δ): 2.25 (3H, s), 6.33 (2H, t, J=2.0 Hz), 6.82 (1H, d, J=6.0 Hz),7.08 (2H, t, J=2.0 Hz), 7.33 (1H, d, J=8.4 Hz), 7.65-7.78 (2H, m), 7.82 (1H, d, J=2.0 Hz), 7.90-8.05 (2H, m), 8.52-8.70 (1H, m), 8.91 (2H, s), 9.23 (1H, s), 9.39 (1H, s).

### EXAMPLE 34

A solution of N-formyl-2-amino-1-[3-(quinolin-2-ylamino)-phenyl]-ethanone (0.32 g), xylene (10 ml), acetic acid (2 ml) and 40% methylamine in water (2 ml) was heated at reflux for 2 hours. The reaction mixture was evaporated and the residue was dissolved in ethyl acetate. The solution was washed with weakly basic brine (three times), dried (magnesium sulfate), filtered and evaporated. The residue was purified by a column chromatography (silica gel, dichloromethane/methanol) to give [3-(1-methyl-imidazol-5-yl)-phenyl]-(quinolin-2-yl)-amine as a brown powder (0.18 g).

m.p.: 60-65° C. IR (KBr, cm⁻¹): 3292, 1592 Mass: 301 (m/z, (M+H)⁺) NMR (DMSO-d₆, δ): 3.82 (3H, s), 7.05-7.10 (3H, m), 7.30 (1H, ddd, J=7, 7, 1.5 Hz), 7.41 (1H, dd, J=7.8, 7.8 Hz), 7.55-7.85 (5H, m), 8.08 (1H, d, J=8.9 Hz), 8.61 (1H, dd, J=1, 1 Hz), 9.55 (1H, s).

### EXAMPLE 35

A mixture of 3-(1-methylimidazol-5-yl)aniline (693 mg) and 1-chloroisoquinoline (164 mg) was heated at 150° C. for an hour. After cooling to room temperature, the reaction mixture was dissolved in dichloromethane. The solution was washed with a diluted aqueous sodium hydroxide solution, and then brine. The organic phase was dried with magnesium sulfate, filtered and evaporated. The residue was purified by a column chromatography (silica gel, dichloromethane/methanol). The obtained product was recrystallized from diethyl ether to give [3-(1-methyl-imidazol-5-yl)-phenyl]-(isoquinolin-1-yl)-amine as white crystals (85 mg).

m.p.: 208-209° C. (diethyl ether) IR (KBr, cm⁻¹): 3282, 1593, 800 Mass: 301 (m/z, (M+H)⁺) NMR (DMSO-d₆, δ): 3.74 (3H, s), 7.05-7.13 (2H, m), 7.21 (1H, d, J=5.7 Hz), 7.40 (1H, dd, J=7.9, 7.9 Hz), 7.63-7.82 (5H, m), 8.00-8.10 (2H, m), 8.55 (1H, d, J=8.3 Hz), 9.25 (1H, s).

### EXAMPLE 36

(4-Benzyl-phthalazin-1-yl)-(3-imidazol-1-yl-phenyl)-amine was prepared from 1-benzyl-4-chloro-phthalazine in a manner similar to Example 35.

m.p.: 214-217° C. (diisopropyl ether) IR (KBr, cm⁻¹): 1612, 1568 Mass: 378 (m/z, (M+H)⁺) NMR (DMSO-d₆, δ): 4.57 (2H, s), 7.10-7.40 (7H, m), 7.50 (1H, t, J=8 Hz), 7.68 (1H, s), 7.80-8.05 (3H, m), 8.10-8.20 (2H, m), 8.27 (1H, t, J=1 Hz), 8.60 (1H, d, J=8 Hz), 9.35 (1H, s).

### EXAMPLE 37

N,N′-Di(isoquinolin-1-yl)-butane-1,4-diamine was prepared in a manner similar to Example 35.

m.p.: 189-192° C. (diisopropyl ether) IR (KBr, cm⁻¹): 3398, 1520 Mass: 343 (m/z, (M+H)⁺) NMR (DMSO-d₆, δ): 1.75 (4H, s), 3.54 (4H, d, J=5 Hz), 6.85 (2H, d, J=6 Hz), 7.40-7.75 (8H, m), 7.84 (2H, d, J=6 Hz), 8.22 (2H, d, J=8 Hz).

### EXAMPLE 38

N,N′-Di(isoquinolin-1-yl)-transcyclohexane-1,4-diamine was prepared in a manner similar to Example 35.

m.p.: 278-280° C. (diisopropyl ether) IR (KBr, cm⁻¹): 3419, 1518 Mass: 369 (m/z, (M+H)⁺) NMR (DMSO-d₆, δ): 1.40-1.80 (4H, m), 2.00-2.40 (4H, m), 4.17 (2H, br s), 6.87 (2H, d, J=8 Hz), 7.10 (2H, br d, J=8 Hz), 7.49 (2H, t, J=8 Hz), 7.55-7.75 (4H, m), 7.87 (2H, d, J=6 Hz), 8.33 (2H, d, J=8 Hz).

### EXAMPLE 39

(Indeno[1,2,3-de]phthalazin-3-yl)-[3-(imidazol-1-yl)-phenyl]-amine was prepared from 3-chloro-indeno[1,2,3-de]phthalazine in a manner similar to Example 35.

m.p.: 223-226° C. (diisopropyl ether) IR (KBr, cm⁻¹): 1608 Mass: 362 (m/z, (M+H)⁺) NMR (DMSO-d₆, δ): 7.16 (1H, s), 7.34 (1H, dd, J=8, 1 Hz), 7.40-7.65 (3H, m), 7.71 (1H, d, J=1 Hz), 7.95-8.10 (4H, m), 8.22 (1H, s), 8.30 (1H, d, J=7 Hz), 8.43 (1H, t, J=2 Hz), 8.48 (1H, d, J=8 Hz), 9.78 (1H, s).

### EXAMPLE 40

(Indeno[1,2,3-de]phthalazin-3-yl)-[3-(isoquinolin-1-ylaminomethyl)-phenyl]-amine was prepared from 3-chloro-indeno[1,2,3-de]phthalazine in a manner similar to Example 35.

m.p.: 149-152° C. (diisopropyl ether-ethyl acetate) IR (KBr, cm⁻¹): 1527 Mass: 452 (m/z, (M+H)⁺) NMR (DMSO-d₆, δ): 4.81 (2H, d, J=6 Hz),6.90 (1H, d, J=6 Hz), 7.10 (1H, d, J=8 Hz), 7.32 (1H, t, J=8 Hz), 7.45-7.75 (5H, m), 7.80-8.10 (7H, m), 8.25 (1H, d, J=7 Hz), 8.35 (1H, d, J=8 Hz), 8.44 (1H, d, J=8 Hz), 9.56 (1H, s).

### EXAMPLE 41

N-[3-(Indeno[1,2,3-de]phthalazin-3-ylamino)-phenyl]-benzamidine hydroiodide was prepared from N-(indeno[1,2,3-de]phthalazin-3-yl)-benzene-1,3-diamine in a manner similar to Example 1.

m.p.: 183-186° C. (diisopropyl ether-methanol) IR (KBr, cm⁻¹): 1655 Mass: 414 (m/z, (M⁺−HI+1)) NMR (DMSO-d₆, δ): 7.15 (1H, br d, J=7 Hz), 7.50-7.85 (8H, m), 7.90-8.10 (7H, m), 8.31 (1H, d, J=7 Hz), 8.40-8.60 (2H, m), 9.85 (1H, s).

### EXAMPLE 42

(Indeno[1,2,3-de]phthalazine-3-yl)-[3-(2,3-dimethyl-3H-imidazol-4-yl)phenyl]amine was prepared from 3-chloro-indeno[1,2,3-de]phthalazine in a manner similar to Example 35.

Mass: 390 (m/z, (M+H)⁺), NMR (DMSO-d₆, δ): 2.38 (3H, s), 3.62 (3H, s), 6.91 (1H, s), 7.13 (1H, d, J=7.7 Hz), 7.4-7.6 (3H, m), 7.9-8.1 (4H, m), 8.21 (1H, s), 8.28 (1H, d, J=7.0 Hz), 8.48 (1H, d, J=8.2 Hz), 9.67 (1H, s).

### EXAMPLE 43

(Indeno[1,2,3-d,e]phthalazin-3-yl)-[3-(1-methyl-imidazol-5-yl)-phenyl]-amine as yellow crystals was from 3-chloro-indeno[1,2,3-de]phthalazine prepared in a manner similar to Example 35.

m.p.: 155-157° C. (ethyl acetate) IR (KBr, cm⁻¹): 1539, 1450, 1400 Mass: 376 (m/z, (M+H)⁺) NMR (DMSO-d₆, δ): 3.77 (3H, s), 7.09 (1H, d, J=1.0 Hz), 7.21 (1H, d, J=7.7 Hz), 7.45-7.53 (3H, m), 7.74 (1H, d, J=1.0 Hz), 7.96-8.07 (4H, m), 8.25-8.31 (2H, m), 8.48 (1H, d, J=8.2 Hz), 9.67 (1H, br s).

### EXAMPLE 44

[3-(Imidazol-1-yl)-phenyl]-(isoquinolin-1-yl)-amine as brown crystals was prepared in a manner similar to Example 35.

m.p.: 164-167° C. (methanol) IR (KBr, cm⁻¹): 3313, 1546 Mass: 287 (m/z, (M+H)⁺) NMR (DMSO-d₆, δ): 7.13 (1H, s), 7.20-7.27 (2H, m), 7.46 (1H, dd, J=8.0, 8.0 Hz), 7.67-7.78 (3H, m), 7.82-7.94 (2H, m), 8.05 (1H, d, J=5.7 Hz), 8.17-8.22 (2H, m), 8.55 (1H, d, J=8.3 Hz), 9.37 (1H, br s).

### EXAMPLE 45

[3-(Imidazol-1-yl)-phenyl]-(phthalazin-1-yl)-amine as a brown powder was prepared in a manner similar to Example 35.

m.p.: 135-137° C. (methanol) IR (KBr, cm⁻¹): 1610 Mass: 288 (m/z, (M+H)⁺) NMR (DMSO-d₆, δ): 7.14 (1H, s), 7.29 (1H, br d, J=9.2 Hz), 7.51 (1H, dd, J=8.0, 8.0 Hz), 7.68 (1H, br s), 7.95-8.05 (4H, m), 8.20 (1H, s), 8.24 (1H, dd, J=2.0, 2.0 Hz), 8.60 (1H, d, J=7.2 Hz), 9.20 (1H, s), 9.39 (1H, s).

### EXAMPLE 46

N-(Indeno[1,2,3-de]phthalazin-3-yl)-N′-(isoquinolin-1-yl)-butane-1,4-diamine was prepared from N-(indeno[1,2,3-de]phthalazin-3-yl)-butane-1,4-diamine in a manner similar to Example 35.

m.p.: 95-105° C. (diisopropyl ether) IR (KBr, cm⁻¹): 1541 Mass: 418 (m/z, (M+H)⁺) NMR (DMSO-d₆, δ): 1.60-2.00 (4H, m), 3.50-3.80 (4H, m), 6.84 (1H, d, J=6 Hz), 7.40-8.40 (15H, m).

### EXAMPLE 47

To a solution of 3-chloro-5-(1,2-dimethyl-1H-imidazol-5-yl)aniline (3.0 g) in tetrahydrofuran (60 ml) was added a 1.5M solution of n-butyl lithium in n-hexane (9 ml) dropwise with stirring at −5° C. followed by stirring for additional 30 minutes at the same temperature. To the reaction mixture was added 4-chloro-5,6-dihydrobenzo[h]quinazoline (2.93 g) and the stirring was continued for 40 hours at ambient temperature. The reaction mixture was evaporated and the residue was dissolved in 0.3N-hydrochloric acid (500 ml). The mixture was washed with dichloromethane (200 ml×3), neutralized with a 1N aqueous solution of sodium hydroxide and extracted with dichloromethane (200 ml×3). The combined organic extracts were dried over magnesium sulfate and filtered. After evaporation of the solvent, the residue was chromatographed on a silica gel eluting with a mixture of dichloromethane and methanol. The obtained product was then triturated with a mixture of ethyl acetate and diisopropyl ether to give N-[3-chloro-5-(1,2-dimethyl-1H-imidazol-5-yl)phenyl]-5,6-dihydrobenzo[h]quinazolin-4-amine (1.5 g) as colorless crystals.

Mass: 402 (m/z, (M+H)⁺) NMR(DMSO-d₆, δ): 2.37 (3H, s), 2.8-3.1 (4H, m), 3.60 (3H, s), 6.97 (1H, s), 7.14 (1H, dd, J=1.6 Hz, 1.6 Hz), 7.3-7.5 (3H, m), 7.81 (1H, dd, J=1.6 Hz, 1.6 Hz), 7.96 (1H, dd, J=1.9 Hz, 1.9 Hz), 8.1-8.3 (1H, m), 8.65 (1H, s), 8.86 (1H, br s).

### EXAMPLE 48

N-[3-(1,2-Dimethyl-1H-imidazol-5-yl)-5-fluorophenyl]-5,6-dihydrobenzo[h]quinazolin-4-amine was obtained in a manner similar to Example 1.

Mass: 386 (m/z, (M+H)⁺) NMR(DMSO-d₆, δ): 2.37 (3H, s), 2.8-3.1 (4H, m), 3.61 (3H, s), 6.9-7.0 (1H, m), 6.95 (1H, s), 7.3-7.5 (3H, m), 7.67 (1H, br s), 7.7-7.9 (1H, m), 8.1-8.3 (1H, m), 8.65 (1H, s), 8.88 (1H, br s).

### EXAMPLE 49

A mixture of 4-chloro-5,6-dihydrobenzo[h]quinazoline (30 g), 3-(1,2-dimethyl-1H-imidazol-5-yl)aniline (25.9 g) and 1,3-dimethyl-2-imidazolidinone (90 ml) was heated for an hour at 200° C. After cooling, a 1N aqueous solution of sodium hydroxide (140 ml) and water (500 ml) were added to the reaction mixture and the resultant mixture was extracted with ethyl acetate (3×300 ml). The combined extracts were washed with an aqueous saturated solution of ammonium chloride (2×400 ml), an aqueous saturated solution of sodium hydrogencarbonate (300 ml) and brine (200 ml). The organic layer was dried over magnesium sulfate, decolorized by activated charcoal powder and then filtered. After evaporation of the solvent, the residue was triturated with a mixture of ethyl acetate and diisopropyl ether, and chromatographed on silica gel eluting with a mixture of dichloromethane and methanol. The obtained product was then triturated with ethyl acetate twice to give N-[3-(1,2-dimethyl-1H-imidazol-5-yl)phenyl]-5,6-dihydrobenzo[h]quinazolin-4-amine (15.5 g) as colorless crystals.

Mass: 368 (m/z, (M+H)⁺) NMR(DMSO-d₆, δ): 2.36 (3H, s), 2.8-3.1 (4H, m), 3.58 (3H, s), 6.88 (1H, s), 7.10 (1H, d, J=7.7 Hz), 7.3-7.5 (4H, m), 7.72 (1H, d, J=8.0 Hz), 7.8-7.9 (1H, m), 8.1-8.3 (1H, m), 8.58 (1H, s), 8.75 (1H, br s).

### EXAMPLE 50

The following compounds described in (1) to (7) were obtained in a manner similar to Example 49.

(1) N-[3-(4,5-Dimethyl-1H-imidazol-1-yl)phenyl]-5,6-dihydrobenzo[h]quinazolin-4-amine

Mass: 368 (m/z, (M+H)⁺) NMR(DMSO-d₆, δ): 2.12 (3H, s), 2.14 (3H, s), 2.8-3.1 (4H, m), 7.05 (1H, d, J=8.9 Hz), 7.3-7.5 (3H, m), 7.47 (1H, t, J=8.0 Hz), 7.65 (1H, s), 7.81 (1H, d, J=8.2 Hz), 7.89 (1H, br s), 8.1-8.3 (1H, m), 8.61 (1H, s), 8.85 (1H, br s).

(2) 3-Chloro-N⁵-(5,6-dihydrobenzo[h]quinazolin-4-yl)-N²-(2-pyridylmethyl)-2,5-pyridinediamine

Mass: 415 (m/z, (M+H)⁺) NMR(DMSO-d₆, δ): 2.7-3.1 (4H, m), 4.68 (2H, d, J=5.7 Hz), 7.05 (1H, t, J=5.7 Hz), 7.2-7.5 (5H, m), 7.6-7.8 (1H, m), 7.98 (1H, d, J=2.3 Hz), 8.1-8.2 (2H, m), 8.48 (1H, s), 8.52 (1H, d, J=4.8 Hz), 8.60 (1H, br s).

(3) N-{6-[(2-Methyl-3-pyridyl)oxy]-3-pyridyl}-5,6-dihydrobenzo[h]quinazolin-4-amine

Mass: 382 (m/z, (M+H)⁺) NMR(DMSO-d₆, δ): 2.33 (3H, s), 2.8-3.1 (4H, m), 7.13 (1H, d, J=8.8 Hz), 7.2-7.5 (4H, m), 7.52 (1H, dd, J=1.4 Hz, 8.1 Hz), 8.1-8.2 (2H, m), 8.2-8.4 (2H, m), 8.52 (1H, s), 8.81 (1H, br s).

(4) N-[3-(4-Methyl-1H-imidazol-1-yl)phenyl]-5,6-dihydrobenzo[h]quinazolin-4-amine

Mass: 354 (m/z, (M+H)⁺) NMR(DMSO-d₆, δ): 2.18 (3H, s), 2.8-3.1 (4H, m), 7.2-7.5 (6H, m), 7.72 (1H, d, J=9.2 Hz), 7.99 (1H, t, J=2.0 Hz), 8.06 (1H, s), 8.1-8.3 (1H, m), 8.62 (1H, s), 8.82 (1H, br s).

(5) N-[3-(1H-1,2,4-Triazol-1-yl)phenyl]-5,6-dihydrobenzo[h]quinazolin-4-amine

Mass: 341 (m/z, (M+H)⁺) NMR(DMSO-d₆, δ): 2.9-3.1 (4H, m), 7.3-7.6 (5H, m), 7.7-7.9 (1H, m), 8.1-8.3 (1H, m), 8.26 (1H, s), 8.3-8.4 (1H, m), 8.63 (1H, s), 8.93 (1H, br s), 9.27 (1H, s).

(6) N-[3-(5-Pyrimidinyl)phenyl]-5,6-dihydrobenzo[h]quinazolin-4-amine

Mass: 352 (m/z, (M+H)⁺) NMR(DMSO-d₆, δ): 2.9-3.1 (4H, m), 7.3-7.6 (5H, m), 7.8-8.0 (1H, m), 8.1-8.3 (2H, m), 8.61 (1H, s), 8.82 (1H, br s), 9.13 (2H, s), 9.21 (1H, s).

(7) N-[4-Methyl-3-(5-pyrimidinyl)phenyl]-5,6-dihydrobenzo[h]quinazolin-4-amine

Mass: 366 (m/z, (M+H)⁺) NMR(DMSO-d₆, δ): 2.25 (3H, s), 2.8-3.1 (4H, m), 7.2-7.5 (4H, m), 7.67 (1H, d, J=2.2 Hz), 7.79 (1H, dd, J=2.3 Hz, 8.3 Hz), 8.1-8.3 (1H, m), 8.55 (1H, s), 8.72 (1H, br s), 8.90 (2H, s), 9.23 (1H, s).

### EXAMPLE 51

A mixture of 3-(1,2-dimethyl-1H-imidazol-5-yl)aniline (94 mg), 4-chloro-5,6-dihydrothieno[2,3-h]quinazoline (112 mg) and 1,3-dimethyl-2-imidazolidinione (1 ml) was heated for 3 hours at 190° C. After cooling, the reaction mixture was dissolved in 0.5N-hydrochloric acid (20 ml) and washed with dichloromethane (20 ml×3). The mixture was neutralized with a 1N aqueous solution of sodium hydroxide and extracted with dichloromethane (20 ml×2). The combined organic extracts were dried over magnesium sulfate, decolorized by activated charcoal powder and then filtered. After evaporation of the solvent, the residue was chromatographed on silica gel eluting with a mixture of dichloromethane and methanol. The obtained product was triturated with ethyl acetate to give N-[3-(1,2-dimethyl-1H-imidazol-5-yl)phenyl]-5,6-dihydrothieno[2,3-h]quinazolin-4-amine (30 mg) as colorless crystals.

Mass: 374 (m/z, (M+H)⁺) NMR(DMSO-d₆, δ): 2.36(3H, s), 3.0-3.2(4H, m), 3.58(3H, s), 6.87(1H, s), 7.08(1H, d, J=7.8 Hz), 7.39(1H, t, J=7.8 Hz), 7.43(1H, d, J=5.2 Hz), 7.51(1H, d, J=5.2 Hz), 7.6-7.8(1H, m), 7.78(1H, t, J=1.7 Hz), 8.49(1H, s), 8.66(1H, br s).

### EXAMPLE 52

N-[3-(1,2-Dimethyl-1H-imidazol-5-yl)phenyl]-5,6-dihydrothieno[3,2-h]quinazolin-4-amine was obtained in a manner similar to Example 5.

Mass: 374 (m/z, (M+H)⁺) NMR(DMSO-d₆, δ): 2.36 (3H, s), 2.9-3.1 (4H, m), 3.58 (3H, s), 6.87 (1H, s), 7.0-7.2 (2H, m), 7.39 (1H, t, J=7.9 Hz), 7.6-7.9 (3H, m), 8.42 (1H, s), 8.68 (1H, br s).

### EXAMPLE 53

A solution of 3-(4,5-dimethyl-1H-imidazol-1-yl)aniline (II) (63 mg) in tetrahydrofuran (2 ml) was added a 1.5M solution in n-hexane of n-butyl lithium (0.34 ml) dropwise under stirring at 0° C. After stirring for additional 30 min at the same temperature, 4-chloro-5,6-dihydrothieno [2,3-h]quinazoline (I) (50 mg) was added to the reaction mixture and the stirring was continued for 4 hours at ambient temperature. The reaction mixture was evaporated and the residue was dissolved in 0.3N-hydrochloric acid (30 ml). The mixture was washed with dichloromethane (20 ml×3), neutralized with a 1N aqueous solution of sodium hydroxide, and extracted with dichloromethane (20 ml×3). The combined organic extracts were dried over magnesium sulfate, decolorized by activated charcoal powder, and filtered. After evaporation of the solvent, the residue was chromatographed on silica gel eluting with 1, 2 and 4% of methanol in dichloromethane. The obtained product was triturated with ethyl acetate to give N-[3-(4,5-dimethyl-1H-imidazol-1-yl)phenyl]-5,6-dihydrothieno[2,3-h]quinazolin-4-amine (32 mg) as crystals.

Mass: 374 (m/z, (M+H)⁺) NMR(DMSO-d₆, δ): 2.12 (3H, s), 2.13 (3H, s), 2.9-3.3 (4H, m), 7.0-7.1 (1H, m), 7.4-7.6 (3H, m), 7.64 (1H, s), 7.7-7.9 (2H, m), 8.53 (1H, s), 8.77 (1H, br s).

### EXAMPLE 54

To a suspension of 2-bromo-1-(5-chloro-2-methoxyphenyl)-ethanone (0.12 g) in ethanol (5 ml) was added 3-(imidazol-1-yl)phenyl-thiourea (100 mg), and the mixture was heated for an hour at 90° C. After cooling to ambient temperature, the reaction mixture was taken up into a mixture of ethyl acetate and a 10% aqueous potassium carbonate solution. The separated organic layer was washed with brine, dried over potassium carbonate and evaporated under reduced pressure. The residue was triturated with diisopropyl ether to give [4-(5-chloro-2-methoxyphenyl)-thiazol-2-yl]-[3-(imidazol-1-yl)phenyl]amine (128 mg).

APCI-mass: 383 (m/z, [M+H]⁺) NMR(DMSO-d₆, δ): 3.94(3H, s), 7.10-7.30(3H, m), 7.30-7.50(3H, m), 7.55(1H, s), 7.70(1H, s), 8.15(1H, d, J=2.7 Hz), 8.22(1H, s), 8.36(1H, s)

### EXAMPLE 55

To a solution of [4-(5-chloro-2-methoxyphenyl)-thiazol-2-yl]-[3-(imidazol-1-yl)phenyl]amine (80 mg) in dichloromethane (1 ml) was added a 1M solution of boron tribromide in dichloromethane (2 ml) at ambient temperature. After stirring for 3 hours at ambient temperature, the mixture was evaporated under reduced pressure. The residue was taken up into a mixture of ethyl acetate and water, and pH of the mixture was adjusted to around 6 with an aqueous sodium hydrogencarbonate solution. The separated organic layer was washed with brine and evaporated to give [4-(5-chloro-2-methoxyphenyl)-thiazol-2-yl]-[3-(imidazol-1-yl)phenyl]amine hydrobromide (18 mg).

APCI-mass: 369 (m/z, free form of [M+H]⁺) NMR(DMSO-d₆, δ): 6.95(1H, d, J=8.6 Hz), 7.15-7.35(3H, m), 7.35-7.55(2H, m), 7.61(1H, s), 7.90(1H, s), 8.02(1H, d, J=2.7 Hz), 8.22(1H, s), 8.40(1H, s), 10.64(1H, s), 10.86(1H, s).

### EXAMPLE 56

To a suspension of 2-bromo-1-(2-chlorophenyl)ethanone (85.6 mg) in ethanol (5 ml) was added 3-(imidazol-1-yl)phenylthiourea (80 mg), and the mixture was heated for an hour at 90° C. After cooling to ambient temperature, the reaction mixture was taken up into a mixture of ethyl acetate and a 10% aqueous potassium carbonate solution. The separated organic layer was washed with brine, dried over potassium carbonate and evaporated under reduced pressure. The residue was triturated with methanol to give [4-(2-chloro-phenyl)thiazol-2-yl]-[3-(imidazol-1-yl)phenyl]amine (81.7 mg).

APCI-mass: 353 (m/z, [M+H]⁺) NMR(DMSO-d₆, δ): 7.13(1H, s), 7.20(1H, d, J=7.7 Hz), 7.30-7.62(6H, m), 7.65(1H, s), 7.92(1H, dd, J=2.2, 7.2 Hz), 8.10-8.22(2H, m), 10.57(1H, s)

### EXAMPLE 57

To a suspension of 2-bromo-1-(4-chlorophenyl)ethanone (85.6 mg) in ethanol (5 ml) was added 3-(imidazol-1-yl)phenylthiourea (80 mg), and the mixture was heated for an hour at 90° C.

After cooling to ambient temperature, the reaction mixture was taken up into a mixture of ethyl acetate and a 10% aqueous potassium carbonate solution. The separated organic layer was washed with brine, dried over potassium carbonate and evaporated under reduced pressure. The residue was triturated with methanol to give [4-(4-chlorophenyl)thiazol-2-yl]-[3-(imidazol-1-yl)phenyl]amine (79.6 mg).

APCI-mass: 353 (m/z, [M+H]⁺) NMR(DMSO-d₆, δ): 7.16(1H, s), 7.22(1H, d, J=7.7 Hz), 7.40-7.58(4H, m), 7.60-7.75(2H, m), 7.90-8.02(3H, m), 8.20(1H, s), 10.58(1H, s).

### EXAMPLE 58

To a suspension of 2-bromo-1-(3-chlorophenyl)ethanone (85.6 mg) in ethanol (5 ml) was added 3-(imidazol-1-yl)phenylthiourea (80 mg), and the mixture was heated for an hour at 90° C. After cooling to ambient temperature, the reaction mixture was taken up into a mixture of ethyl acetate and a 10% aqueous potassium carbonate solution. The separated organic layer was washed with brine, dried over potassium carbonate and evaporated under reduced pressure. The residue was triturated with methanol to give [4-(3-chlorophenyl)thiazol-2-yl]-[3-(imidazol-1-yl)phenyl]amine (76.3 mg).

APCI-mass: 353 (m/z, [M+H]⁺) NMR(DMSO-d₆, δ): 7.15(1H, s), 7.23(1H, d, J=7.7 Hz), 7.30-7.62(5H, m), 7.69(1H, s), 7.89(1H, d, J=7.5 Hz), 7.99(1H, s), 8.18(1H, s), 8.21(1H, s), 10.61(1H, s).

### EXAMPLE 59

To a suspension of 2-bromo-1-(5-chlorothiophen-2-yl)ethanone (87 mg) in ethanol (5 ml) was added 3-(imidazol-1-yl)phenylthiourea (80 mg), and the mixture was heated for an hour at 90° C. After cooling to ambient temperature, the reaction mixture was taken up into a mixture of ethyl acetate and a 10% aqueous potassium carbonate solution. The separated organic layer was washed with brine, dried over potassium carbonate and evaporated under reduced pressure. The residue was triturated with methanol to give [4-(5-chlorothiophen-2-yl)thiazol-2-yl]-[3-(imidazol-1-yl)phenyl]amine (86.0 mg).

APCI-mass: 359 (m/z, [M+H]⁺) NMR(DMSO-d₆, δ): 7.06-7.18(2H, m), 7.18-7.29(1H, m), 7.32(1H, s), 7.40(1H, d, J=3.9 Hz), 7.43-7.50(2H, m), 7.69(1H, s), 8.15(1H, s), 8.20(1H, s), 10.65(1H, s).

### EXAMPLE 60

To a suspension of bromo-phenylacetaldehyde (95 mg) in ethanol (2 ml) was added 3-(imidazol-1-yl)phenylthiourea (80 mg), and the mixture was heated for 1.5 hours at 90° C. After cooling to ambient temperature the reaction mixture was taken up into a mixture of ethyl acetate and a 10% aqueous potassium carbonate solution. The separated organic layer was washed with brine, dried over potassium carbonate and evaporated under reduced pressure. The residue was chromatographed on silica gel eluting with a mixture of dichloromethane and methanol (0-8% v/v) to give [3-(imidazol-1-yl)phenyl]-(5-phenylthiazol-2-yl)amine (34 mg).

APCI-mass: 319 (m/z, [M+H]⁺) NMR(DMSO-d₆, δ): 7.05-7.61(9H, m), 7.65(1H, s), 7.75(1H, s), 7.99(1H, s), 8.17(1H, s), 10.60(1H, s).

### EXAMPLE 61

To a suspension of 2-bromo-1-phenylethanone (36 mg) in ethanol (1 ml) was added 3-(imidazol-1-yl)phenylthiourea (40 mg), and the mixture was heated for an hour at 90° C. After cooling to ambient temperature, the reaction mixture was taken up into a mixture of ethyl acetate and a 10% aqueous potassium carbonate solution. The separated organic layer was washed with brine, dried over potassium carbonate and evaporated under reduced pressure. The residue was triturated with methanol to give (4-phenylthiazol-2-yl)-[3-(imidazol-1-yl)phenyl]amine (37 mg).

APCI-mass: 319 (m/z, [M+H]⁺).

### EXAMPLE 62

A solution of [3-(2,3-dimethyl-3H-imidazol-4-yl)phenyl]thiourea (0.2 g) and bromo-phenylacetaldehyde (0.24 g) in ethanol (3 ml) was heated under reflux for 30 minutes. After cooling to ambient temperature, the reaction mixture was taken up into a mixture of ethyl acetate and a 10% aqueous potassium carbonate solution. The separated organic layer was washed with brine, dried over potassium carbonate and evaporated under reduced pressure. The residue was chromatographed on silica gel eluting with a mixture of dichloromethane and methanol (0-8% v/v) to give [3-(2,3-dimethyl-3H-imidazol-4-yl)phenyl]-(5-phenylthiazol-2-yl)amine (91.7 mg).

APCI-mass: 347 (m/z, [M+H]⁺) NMR(DMSO-d₆, δ): 2.30(3H, s), 3.56(3H, s), 6.80-7.80(11H, m), 10.44(1H, s).

### EXAMPLE 63

To a solution of [3-(2,3-dimethyl-3H-imidazol-4-yl)phenyl]-thiourea (80 mg) in ethanol (2 ml) was added 1-chloro-3,4-dihydro-1H-naphthalen-2-one (176 mg), and the mixture was heated for 2 hours at 80° C. After evaporation of the solvent, the residue was taken up into a mixture of ethyl acetate and an aqueous potassium carbonate solution. The separated organic layer was washed with brine, dried over potassium carbonate and evaporated under reduced pressure. The residue was chromatographed on silica gel eluting with a mixture of dichloromethane and methanol (0-4% V/V). The obtained product was crystallized from methanol to give (4,5-dihydronaphtho[2,1-d]thiazol-2-yl)-[3-(2,3-dimethyl-3H-imidazol-4-yl)phenyl]amine (49.4 mg).

APCI-mass: 373.33 (m/z, (M+H)⁺) NMR(DMSO-d₆, δ): 2.36(3H, s), 2.75-2.90(2H, m), 2.90-3.09(2H, m), 3.58(3H, s), 6.88(1H, s), 6.97-7.29(5H, m), 7.39(1H, t, J=7.9 Hz), 7.58(1H, d, J=7.9 Hz), 7.81(1H, s), 10.47(1H, s).

### EXAMPLE 64

To a solution of [3-(4,5-dimethylimidazol-1-yl)phenylthiourea (0.15 g) in ethanol (2 ml) was added bromo-phenylacetaldehyde (121 mg), and the mixture was heated under reflux for 2 hours. After evaporation of the solvent, the residue was taken up into a mixture of dichloromethane and an aqueous potassium carbonate solution. The separated organic layer was washed with brine, dried over potassium carbonate and evaporated under reduced pressure. The residue was chromatographed on silica gel eluting with a mixture of dichloromethane and methanol (0-3% V/V) to give [3-(4,5-dimethylimidazol-1-yl)phenyl]- (5-phenylthiazol-2-yl) amine (63.8 mg).

APCI-mass: 347.47 (m/z, (M+H)⁺) NMR(DMSO-d₆, δ): 2.11(6H, s), 6.97(1H, d, J=7.9 Hz), 7.23-7.34(1H, m), 7.34-7.49(3H, m), 7.49-7.63(3H, m), 7.65(1H, s), 7.72 (1H, s), 7.80-7.90(1H, m), 10.60(1H, s).

### EXAMPLE 65

To a solution of [3-(4,5-dimethylimidazol-1-yl)phenylthiourea (0.3 g) in ethanol (5 ml) was added bromo-(2-methoxy)phenylacetaldehyde (1.28 g) and the mixture was heated for 2 hours at 80° C. After evaporation of the solvent, the residue was taken up into a mixture of dichloromethane and an aqueous potassium carbonate solution. The separated organic layer was washed with brine, dried over potassium carbonate and evaporated under reduced pressure. The residue was chromatographed on silica gel eluting with a mixture of dichloromethane and methanol (0-3.5% V/V) to give [3-(4,5-dimethylimidazol-1-yl)phenyl]-[5-(2-methoxyphenyl)thiazol-2-yl]amine (57.7 mg).

APCI-mass: 377.40 (m/z, (M+H)⁺) NMR(DMSO-d₆, δ): 2.12(3H, s), 2.14(3H, s), 3.90(3H, s), 6.90-7.20(3H, m), 7.20-7.35(1H, m), 7.35-7.70(3H, m), 7.75-7.95(3H, m), 10.50(1H, s).

### EXAMPLE 66

To a solution of [3-(4,5-dimethylimidazol-1-yl)phenyl]thiourea (80 mg) in ethanol (5 ml) was added 1-chloro-3,4-dihydro-1H-naphthalen-2-one (235 mg), and the mixture was heated for 2 hours at 80° C. After evaporation of the solvent, the residue was taken up into a mixture of ethyl acetate and an aqueous potassium carbonate solution. The separated organic layer was washed with brine, dried over potassium carbonate and evaporated under reduced pressure. The residue was chromatographed on silica gel eluting with a mixture of dichloromethane and methanol (0-2% V/V). The obtained product was crystallized from methanol to give (4,5-dihydronaphtho[2,1-d]thiazol-2-yl)-[3-(4,5-dimethylimidazol-1-yl)phenyl]amine (32.8 mg).

APCI-mass: 373.33 (m/z, (M+H)⁺) NMR(DMSO-d₆, δ): 2.13(6H, s), 2.73-2.91(2H, m), 2.91-3.10(2H, m), 6.90-7.32(5H, m), 7.45(1H, t, J=8.0 Hz), 7.53-7.64(1H, m), 7.66(1H, s), 7.85(1H, s), 10.62(1H, s).

### EXAMPLE 67

To a solution of [3-(4,5-dimethylimidazol-1-yl)phenyl]thiourea (100 mg) in ethanol (3 ml) was added 1-chloroindan-2-one (338 mg), and the mixture was heated for 2 hours at 80° C. After evaporation of the solvent, the residue was taken up into a mixture of ethyl acetate and an aqueous potassium carbonate solution. The separated organic layer was washed with brine, dried over potassium carbonate and evaporated under reduced pressure. The residue was chromatographed on silica gel eluting with a mixture of dichloromethane and methanol (0-5% V/V). The obtained product was crystallized from methanol to give N-[3-(4,5-dimethyl-1H-imidazol-1-yl)phenyl]-N-(4H-indeno[2,1-d][1,3]thiazol-2-yl)amine (56.3 mg).

APCI-mass: 359.33 (m/z, (M+H)⁺) NMR(DMSO-d₆, δ): 2.12(6H, s), 3.74(2H, s), 6.98(1H, d, J=8.7 Hz), 7.12(1H, t, J=7.3 Hz), 7.28(1H, t, J=7.3 Hz), 7.37-7.59(3H, m), 7.59-7.75(2H, m), 7.86(1H, s), 10.71(1H, s).

### EXAMPLE 68

To a solution of 2-indanone (0.35 g) in dichloromethane (0.2 ml) was added sulfuryl chloride (0.264 ml) at ambient temperature. After stirring for 12 hours at ambient temperature, the reaction mixture was diluted with a mixture of ethyl acetate and water, and pH of the mixture was adjusted around 7 with an aqueous potassium carbonate solution. The separated organic layer was dried over magnesium sulfate and evaporated in vacuo. The residue was dissolved in ethanol (2 ml) to give a crude 1-chloroindanone solution. To this solution was added [6-(2-methylpyridin-3-yloxy)pyridin-3-yl]thiourea (138 mg), and the mixture was heated at 90° C. for 2 hours. After evaporation of the solvent in vacuo, the residue was taken up into a mixture of ethyl acetate and an aqueous solution of sodium hydroxide. The separated organic layer was dried over magnesium sulfate and evaporated in vacuo. The obtained residue was chromatographed on silica gel eluting with a mixture of dichloromethane and methanol (0-4% V/V). The obtained product was crystallized from dichloromethane to give N-[6-(2-methylpyridin-3-yloxy)pyridin-3-yl]-N-(4H-indeno[2,1-d][1,3]thiazol-2-yl)amine (53.5 mg).

APCI-mass: 373.20 (m/z, (M+H)⁺) NMR(DMSO-d₆, δ): 2.33(3H, s), 3.71(2H, s), 7.08-7.22(2H, m), 7.22-7.35(2H, m), 7.38(1H, d, J=6.8 Hz), 7.43-7.57(2H, m), 8.26-8.45(3H, m), 10.56(1H, s).

### EXAMPLE 69

To a suspension of 5-chloro-N-(4H-indeno[2,1-d][1,3]thiazol-2-yl)benzene-1,3-diamine hydrochloride (80 mg) in 2-propanol (2 ml) was added methyl benzenecarbimidothioate hydroiodide (255 mg), and the mixture was heated for 3 hours at 100° C. The reaction mixture was diluted with ethyl acetate, washed with an aqueous potassium carbonate solution and dried over potassium carbonate. After evaporation of the solvent in vacuo, the resultant precipitate was collected by filtration and washed with methanol and dichloromethane to give N-[3-chloro-5-(4H-indeno[2,1-d][1,3]thiazol-2-ylamino)phenyl]benzamidine (55 mg).

APCI-mass: 417.20, 419.20 (m/z, (M+H)⁺) NMR(DMSO-d₆, δ): 3.75(2H, s), 6.45(1H, s), 6.52(2H, brs), 7.04(1H, s), 7.12(1H, dt, J=1.3, 7.4 Hz), 7.28(1H, t, J=7.4 Hz), 7.33-7.62(6H, m), 7.90-8.10(2H, m), 10.52(1H, s).

### EXAMPLE 70

A mixture of 3-(2,3-dimethyl-3H-imidazol-4-yl)phenylamine (173 mg) and 4-chloro-6-phenylpyrimidine (88 mg) was heated for 10 minutes at 190° C. The reaction mixture was taken up into a mixture of ethyl acetate and a 10% aqueous potassium carbonate solution. The separated organic layer was washed with brine, dried over potassium carbonate and evaporated under reduced pressure. The residue was purified by a gel permeation chromatography (JAIGEI-1H/2H) eluting with 0.5% triethylamine in chloroform to give [3-(2,3-dimethyl-3H-imidazol-4-yl)phenyl]-(6-phenylpyrimidin-4-yl)amine (40 mg)

APCI-mass: 342 (m/z, [M+H]⁺) NMR(DMSO-d₆, δ): 2.37(3H, s), 3.58(3H, s), 6.89(1H, s), 7.08(1H, d, J=7.8 Hz), 7.27(1H, s), 7.42(1H, t, J=7.8 Hz), 7.49-7.74(4H, m), 7.80-7.88(1H, m), 7.98-8.10(2H, m), 8.73(1H, s), 9.80(1H, s).

### EXAMPLE 71

A mixture of 3-(2,3-dimethyl-3H-imidazol-4-yl)phenylamine (152 mg) and 4-chloro-6-(thiophen-2-yl)pyrimidine (80 mg) was heated for 7 minutes at 190° C. The reaction mixture was taken up into a mixture of ethyl acetate and a 10% aqueous potassium carbonate solution. The separated organic layer was washed with brine, dried over potassium carbonate and evaporated under reduced pressure. The residue was purified by a gel permeation chromatography (JAIGEI-1H/2H) eluting with 0.5% triethylamine in chloroform to give [3-(2,3-dimethyl-3H-imidazol-4-yl)phenyl]-[6-(thiophen-2-yl)pyrimidin-4-yl]amine (29 mg).

APCI-mass: 348.53 (m/z, [M+H]⁺) NMR(DMSO-d₆, δ): 2.38(3H, s), 3.58(3H, s), 6.93(1H, s), 7.01-7.24(3H, m), 7.42(1H, t, J=7.8 Hz), 7.59-7.70(1H, m), 7.70-7.84(3H, m), 8.60(1H, s), 9.81(1H, s).

### EXAMPLE 72

A mixture of 3-(4,5-dimethylimidazol-1-yl)phenylamine(0.299 g) and 4-chloro-6-(thiophen-2-yl)pyrimidine(157 mg) was heated for 7 minutes at 190° C. The reaction mixture was taken up into a mixture of ethyl acetate and a 10% aqueous potassium carbonate solution. The separated organic layer was washed with brine, dried over potassium carbonate and evaporated under reduced pressure. The residue was purified by a gel permeation chromatography (JAIGEI-1H/2H) eluting with 0.5% triethylamine in chloroform to give [3-(4,5-dimethylimidazol-1-yl)phenyl]-[6-(thiophen-2-yl)pyrimidin-4-yl]amine (21 mg).

APCI-mass: 348.53 (m/z, [M+H]⁺) NMR(DMSO-d₆, δ): 2.12(6H, s), 7.04(1H, dd, J=1.1, 7.8 Hz), 7.16(1H, d, J=1.0 Hz), 7.23(1H, dd, J=3.7, 5.0 Hz), 7.48(1H, t, J=7.8 Hz), 7.59-7.70(2H, m), 7.70-7.86(2H, m), 7.92(1H, t, J=2.0 Hz), 8.63(1H, d, J=1.0 Hz), 9.95(1H, s).

### EXAMPLE 73

A mixture of 3-(2,3-dimethyl-3H-imidazol-4-yl)phenylamine (0.37 g) and 3-benzyl-6-chloropyridazine(0.2 g) was heated for 30 hours at 190° C. The reaction mixture was taken up into a mixture of ethyl acetate and a 10% aqueous potassium carbonate solution. The separated organic layer was washed with brine, dried over potassium carbonate and evaporated under reduced pressure. The residue was purified by a gel permeation chromatography (JAIGEI-1H/2H) eluting with 0.5% triethylamine in chloroform to give (6-benzylpyridazin-3-yl)-[3-(2,3-dimethyl-3H-imidazol-4-yl)phenyl]amine (6.3 mg).

APCI-mass: 356 (m/z, [M+H]⁺) NMR(DMSO-d₆, δ): 2.32(3H, s), 3.58(3H, s), 4.13(2H, s), 7.07(1H, d, J=9.1 Hz), 7.15-7.44(10H, m), 8.07(1H, s), 9.18(1H, s).

### EXAMPLE 74

To a solution of N-[3-(2,3-dimethyl-3H-imidazol-4-yl)phenyl]guanidine dihydrochloride (115 mg) in ethanol (5 ml) were added 2-dimethylaminomethylene-3,4-dihydro-2H-naphthalen-1-one (77 mg) and pyridine(154 μl), and the mixture was heated for 8 hours at 120° C. After evaporation of the solvent under reduced pressure, the residue was taken up into a mixture of dichloromethane and an aqueous solution of sodium hydroxide. The separated organic layer was washed with brine, dried over potassium carbonate and evaporated. The residue was chromatographed on silica gel eluting with a mixture of dichloromethane and methanol (0-5%, v/v) to give (5,6-dihydrobenzo[h]quinazolin-2-yl)-[3-(2,3-dimethyl-3H-imidazol-4-yl)phenyl]amine (85.3 mg).

APCI-mass: 368 (m/z, [M+H]⁺) NMR(DMSO-d₆, δ): 2.36(3H, s), 2.70-2.88(2H, m), 2.88-3.05(2H, m), 3.56(3H, s). 6.87(1H s), 6.98(1H, d, J=7.8 Hz), 7.29-7.55(4H, m), 7.86(1H, dd, J=1.2, 8.2 Hz), 7.95(1H, d, J=1.7 Hz), 8.22(1H, dd, J=1.2, 6.8 Hz), 8.41(1H, s), 9.65(1H, s).

### EXAMPLE 75

To a solution of N-[3-(2,3-dimethyl-3H-imidazol-4-yl)phenyl]guanidine dihydrochloride (200 mg) in ethanol (5 ml) were added 2-dimethylaminomethylene-5-methoxy-3,4-dihydro-2H-naphthalen-1-one (153 mg) and pyridine (0.268 ml), and the mixture was heated for 12 hours at 100° C. After evaporation of the solvent under reduced pressure, the residue was taken up into a mixture of ethyl acetate and an aqueous solution of sodium hydroxide. The separated organic layer was washed with brine, dried over potassium carbonate and evaporated. The residue was chromatographed on silica gel eluting with a mixture of dichloromethane and methanol (0-6%, v/v) to give [3-(2,3-dimethyl-3H-imidazol-4-yl)phenyl]-(7-methoxy-5,6-dihydrobenzo[h]quinazolin-2-yl)amine (31.8 mg).

APCI-mass: 398.47 (m/z, (M+H)⁺) NMR(DMSO-d₆, δ): 2.36(3H, s), 2.65-2.95(4H, m), 3.56(3H, s), 3.86(3H, s), 6.87(1H, s), 6.98(1H, d, J=7.4 Hz), 7.16(1H, d, J=8.0 Hz), 7.38(2H, t, J=8.0 Hz), 7.78-7.92(2H, m), 7.96(1H, s), 8.41(1H, s), 9.63(1H, s).

### EXAMPLE 76

To a solution of 3-aminopyridine (0.15 g) in tetrahydrofuran (5 ml) was added dropwise a 1.54M solution of n-butyl lithium in n-hexane (0.47 ml) at 0° C. followed by stirring for 15 minutes at 0° C. To the mixture was added a solution of 2-methanesulfinyl-7-methoxy-5,6-dihydrobenzo[h]quinazoline (72 mg) in tetrahydrofuran (5 ml) at 0° C. The reaction mixture was stirred for 2 hours at ambient temperature, and was taken up into a mixture of ethyl acetate and water. The separated organic layer was washed with brine and dried over potassium carbonate. After evaporation of the solvent under reduced pressure, the residue was chromatographed on silica gel eluting with 0%-2% methanol in dichloromethane. The obtained product was triturated with diisopropyl ether to give (7-methoxy-5,6-dihydrobenzo[h]quinazolin-2-yl)pyridin-3-ylamine (64 mg).

APCI-mass: 305 (m/z, [M+H]⁺) NMR(DMSO-d₆, δ): 2.70-2.95(4H, m), 3.85(3H, s), 7.16(1H, d, J=7.6 Hz), 7.30-7.50(2H, m), 7.85(1H, d, J=7.4 Hz), 8.15(1H, d, J=4.6 Hz), 8.28(1H, d, J=8.4 Hz), 8.43(1H, s), 8.98(1H, d, J=2.5 Hz), 9.73(1H, s).

### EXAMPLE 77

To a solution of 3-(imidazol-1-yl)phenylamine (0.12 g) in tetrahydrofuran (5 ml) was added dropwise a 1.54M solution of n-butyl lithium in n-hexane (0.46 ml) at 0° C. The mixture was stirred for 15 minutes at 0° C, and a solution of 2-methanesulfinyl-7-methoxy-5,6-dihydrobenzo[h]quinazoline (0.15 g) in tetrahydrofuran (5 ml) was added to the mixture at 0° C. The reaction mixture was stirred for 2 hours at ambient temperature, and was taken up into a mixture of ethyl acetate and water. The separated organic layer was washed with brine and dried over potassium carbonate. After evaporation of the solvent under reduced pressure, the residue was chromatographed on silica gel eluting with 0%-2% methanol in dichloromethane. The obtained product was triturated with diisopropyl ether to give [3-(imidazol-1-yl)phenyl]-(7-methoxy-5,6-dihydrobenzo[h]quinazolin-2-yl) amine (92 mg).

APCI-mass: 370 (m/z, [M+H]⁺) NMR(DMSO-d₆, δ): 2.70-2.95(4H, m), 3.86(3H, s), 7.10-7.29(3H, m), 7.30-7.50(2H, m), 7.65(1H, s), 7.70-7.90(2H, m), 8.16(1H, s), 8.28(1H, s), 8.45(1H, s), 9.79(1H, s).

### EXAMPLE 78

A mixture of 4-chloro-9-methoxy-5,6-dihydrobenzo[h]quinazoline (100 mg) and 3-(4,5-dimethylimidazol-1-yl)phenylamine (152 mg) was heated for 45 minutes at 190° C. After cooling to ambient temperature, the mixture was diluted with a mixture of dichloromethane and a 1N aqueous solution of sodium hydroxide. The separated organic layer was washed in turn with 0.1N-hydrochloric acid (5 ml) and brine and dried over magnesium sulfate. After evaporation of the solvent, the residue was chromatographed on silica gel eluting with a mixture of dichloromethane and methanol (0-5%, v/v) to give [3-(4,5-dimethylimidazol-1-yl)phenyl]-(9-methoxy-5,6-dihydrobenzo[h]quinazolin-4-yl)amine (42.6 mg).

APCI-mass: 398.47 (m/z, (M+H)⁺) NMR(DMSO-d₆, δ): 2.12(3H, s), 2.14(3H, s), 2.91(4H, s), 3.81(3H, s), 6.95-7.11(2H, m), 7.25(1H, d, J=8.3 Hz), 7.47(1H, t, J=8.0 Hz), 7.65(1H, s), 7.73(1H, d, J=2.7 Hz), 7.81(1H, d, J=7.5 Hz), 7.89(1H, s), 8.61(1H, s), 8.85(1H, s).

### EXAMPLE 79

A mixture of 4-chloro-9-methoxy-5,6-dihydrobenzo[h]quinazoline (100 mg) and 3-(4-methylimidazol-1-yl)-phenylamine (140 mg) was heated for 45 minutes at 190° C. After cooling to ambient temperature, the mixture was diluted with a mixture of dichloromethane and a 1N aqueous solution of sodium hydroxide. The separated organic layer was washed in turn with 0.1N-hydrochloric acid (5 ml) and brine and dried over magnesium sulfate. After evaporation of the solvent, the residue was crystallized from methanol to give (9-methoxy-5,6-dihydrobenzo[h]quinazolin-4-yl)-[3-(4-methylimidazol-1-yl)-phenyl]amine (65.6 mg).

APCI-mass: 384.27 (m/z, (M+H)⁺) NMR(DMSO-d₆, δ): 2.18(3H, s), 2.91(4H, s), 3.81(3H, s), 6.99(1H, dd, J=2.7,8.3 Hz), 7.20-7.34(2H, m), 7.34-7.53(2H, m), 7.69-7.80(2H, m), 7.99(1H, s), 8.06(1H, s), 8.62(1H, s), 8.82(1H, s).

### EXAMPLE 80

A mixture of 4-chloro-9-methoxy-5,6-dihydrobenzo[h]quinazoline (100 mg) and 3-(2,3-dimethyl-3H-imidazol-4-yl)phenylamine (152 mg) was heated for 25 minutes at 190° C. After cooling to ambient temperature, the mixture was diluted with a mixture of dichloromethane and a 1N aqueous solution of sodium hydroxide. The separated organic layer was washed in turn with 0.1N-hydrochloric acid (5 ml) and brine and dried over magnesium sulfate. After evaporation of the solvent, the residue was chromatographed on silica gel eluting with a mixture of dichloromethane and methanol (0-5%, v/v), followed by crystallization from methanol to give [3-(2,3-dimethyl-3H-imidazol-4-yl)phenyl]-(9-methoxy-5,6-dihydrobenzo[h]quinazolin-4-yl)amine (29.2 mg).

APCI-mass: 398.40 (m/z, (M+H)⁺) NMR(DMSO-d₆, δ): 2.36(3H, s), 2.91(4H, s), 3.58(3H, s), 3.80(3H, s), 6.87(1H, s), 6.98(1H, dd, J=2.8, 8.3 Hz), 7.09(1H, d, J=7.6 Hz), 7.25(1H, d, J=8.3 Hz), 7.40(1H, t, J=7.8 Hz), 7.69-7.79(2H, m), 7.80(1H, s), 8.58(1H, s), 8.75(1H, s).

### EXAMPLE 81

To a suspension of 3-(1H-imidazol-1-yl)aniline (170 mg) in tetrahydrofuran (3 ml) was added a 1.54M solution of n-butyl lithium in n-hexane (0.65 ml) dropwise at 0° C., and the mixture was stirred for 15 minutes at 0° C. To the mixture was added a solution of 3-chloro-9-methyl-4,5-dihydro[1]benzoxepino[5,4-c]isoxazole (180 mg) in tetrahydrofuran (3 ml) dropwise at0° C. and the mixture was stirred for 48 hours at ambient temperature. The mixture was diluted with ethyl acetate, washed with an aqueous saturated solution of ammonium chloride and brine, dried over magnesium sulfate and evaporated. The residue was purified by a column chromatography on silica gel eluting with 2% methanol in dichloromethane to give N-(3-(1H-imidazol-1-yl)phenyl)-9-methyl-4,5-dihydro[1]benzoxepino[5,4-c]isoxazol-3-amine (27 mg, 10.0%).

APCI-mass: 359 (m/z, (M+H)⁺) NMR(DMSO-d₆, δ): 2.30 (3H, s), 2.82 (2H, t, J=5.1 Hz), 4.25 (2H, t, J=5.1 Hz), 6.96 (1H, d, J=8.3 Hz), 7.1-7.3 (4H, m), 7.34 (1H, s), 7.45 (1H, t, J=8.0 Hz), 7.68 (1H, s), 7.90 (1H, s), 8.19 (1H, s), 9.60 (1H, s).

### EXAMPLE 82

To a suspension of 3-aminopyridine (286 mg) in tetrahydrofuran (5 ml) was added a 1.54M solution of n-butyl lithium in n-hexane (1.57 ml) dropwise at 0° C., and the mixture was stirred for 15 minutes at 0° C. To the mixture was added a solution of 3-chloro-9-methyl-4,5-dihydro[1]benzoxepino[5,4-c]isoxazole (143 mg) in tetrahydrofuran (5 ml) dropwise at 0° C., and the mixture was stirred for 216 hours at ambient temperature. The mixture was diluted with ethyl acetate and washed with an aqueous saturated solution of ammonium chloride, water and brine. The separated organic layer was dried over magnesium sulfate and evaporated. The residue was crystallized from methanol, collected by filtration and dried to give N-(9-methyl-4,5-dihydro[1]benzoxepino[5,4-c]isoxazol-3-yl)-N-(3-pyridyl)amine (50 mg, 28.1%).

APCI-mass: m/z 294 (m/z, (M+H)⁺) NMR(DMSO-d₆, δ): 2.30 (3H, s), 2.83 (2H, t, J=5.2 Hz), 4.25 (2H, t, J=5.2 Hz), 6.96 (1H, d, J=8.3 Hz), 7.18 (1H, dd, J=8.3 Hz, 2.2 Hz), 7.34 (1H, dd, J=8.4 Hz, 4.6 Hz), 7.6-7.7 (1H, m), 7.89 (1H, d, J=1.7 Hz), 8.17 (1H, dd, J=4.6 Hz, 1.7 Hz), 8.50 (1H, d, J=2.5 Hz), 9.59 (1H, s).

### EXAMPLE 83

To a solution of 2-((dimethylamino)methylene)cycloheptanone (251 mg) and N″-(3-(1,2-dimethyl-1H-imidazol-5-yl)phenyl)guanidine dihydrochloride (302 mg) in ethanol (5 ml) was added a 28% solution of sodium methoxide in methanol (0.6 ml), and the mixture was refluxed for 6 hours. The solvent was removed by evaporation and the residue was dissolved in 3N-hydrochloric acid and washed with ethyl acetate. The separated aqueous phase was adjusted to pH 9.5 with a 1N aqueous solution of sodium hydroxide and extracted with ethyl acetate. The separated organic layer was washed with brine, dried over magnesium sulfate and evaporated. The residue was purified with a column chromatography on silica gel eluting with 1-5% methanol in dichloromethane to give N-(3-(1,2-dimethyl-1H-imidazol-5-yl)phenyl)-6,7,8,9-tetrahydro-5H-cyclohepta[d]pyrimidin-2-amine (32 mg, 9.6%).

APCI-MASS: 344 (m/z, (M+H)⁺) NMR(DMSO-d₆, δ): 1.4-1.8 (4H, m), 1.8-2.0 (2H, m), 2.35 (3H, s), 2.6-2.8 (2H, m), 2.8-3.0 (2H, m), 3.56 (3H, s), 6.83 (1H, s), 6.94 (1H, d, J=7.7 Hz), 7.31 (1H, t, J=7.9 Hz), 7.74 (1H, d, J=8.2 Hz), 7.88 (1H, s), 8.18 (1H, s), 9.51 (1H, s).

### EXAMPLE 84

To a solution of 1-[(dimethylamino)methylene]-1,3-dihydro-2H-inden-2-one (374 mg) and N″-(3-(1,2-dimethyl-1H-imidazol-5-yl)phenyl)guanidine dihydrochloride (302 mg) in methanol (5 ml) was added a 28% solution of sodium methoxide in methanol (1 ml), and the mixture was refluxed for 12 hours. The solvent was removed by evaporation and the residue was dissolved in ethyl acetate and washed with water and brine. The separated organic layer was dried over magnesium sulfate and evaporated. The residue was purified by a column chromatography on silica gel eluting with 1-4% methanol in dichloromethane to give N-(3-(1,2-dimethyl-1H-imidazol-5-yl)phenyl)-9H-indeno[2,1-d]pyrimidin-2-amine (42 mg, 11.9%).

APCI-MASS: 354 (m/z, (M+H)⁺) NMR(DMSO-d₆, δ): 2.37 (3H, s), 3.73 (3H, s), 3.99 (2H, s), 6.86 (1H, s), 7.00 (1H, d, J=7.8 Hz), 7.2-7.5 (3H, m), 7.57 (1H, d, J=6.9 Hz), 7.77 (1H, d, J=8.2 Hz), 7.84 (1H, d, J=6.5 Hz), 7.97 (1H, s), 8.99 (1H, s), 10.20 (1H, s).

### EXAMPLE 85

A suspension of 1-bromo-3-(1,2-dimethylimidazol-5-yl)benzene (116 mg), 4,5-dihydro[1]benzoxepino[5,4-c]isoxazol-3-amine (112 mg), sodium tert-butoxide (62 mg), biphenyl-2-yl-di-tert-butylphosphine (11 mg) and tris(dibenzylideneacetone)dipalladium (8 mg) in toluene (1 ml) was stirred for 12 hours at ambient temperature and for an hour at 60° C. The mixture was diluted with ethyl acetate and washed with water and brine. The organic layer was separated, dried over magnesium sulfate and evaporated. The residue was purified by a column chromatography on silica gel eluting with 3% methanol in dichloromethane to give N-[3-(1,2-dimethyl-1H-imidazol-5-yl)phenyl]-4,5-dihydro[1]benzoxepino[5,4-c]isoxazol-3-amine (11 mg, 6.4%).

APCI-MASS: 373 (m/z, (M+H)⁺) NMR(DMSO-d₆, δ): 2.35 (3H, s), 2.85 (2H, t, J=5.1 Hz), 3.55 (1H, s), 4.30 (2H, t, J=5.1 Hz), 6.55 (1H, s), 6.86 (1H, s), 7.0-7.3 (4H, m), 7.3-7.5 (2H, m), 8.10 (1H, dd, J=1.6 Hz, 7.9 Hz), 9.47 (1H, s).

### EXAMPLE 86

A mixture of 3-(1,2-dimethyl-1H-imidazol-5-yl)aniline (500 mg) and 4-chloro-5,6-dihydro[1]benzoxepino[5,4-d]pyrimidine (110 mg) was heated for an hour at 150° C. After cooling, methanol (1 ml) and dichloromethane (1 ml) was added to the reaction mixture. The reaction mixture was diluted with dichloromethane (50 ml) and water (50 ml), 0.1N-hydrochloric acid (20 ml) was added to the mixture and the organic layer was separated. After adding a 0.1N aqueous solution of sodium hydroxide (2 ml), the organic layer was extracted with dichloromethane until the product was obtained. The combined organic phases were washed with a dilute aqueous solution of sodium hydroxide and brine, dried over magnesium sulfate and filtered. After evaporation of the solvent, the residue was purified by a silica gel column chromatography eluting with a mixture of dichloromethane and methanol. The obtained product was recrystallized from diethyl ether to give N-[3-(1,2-dimethyl-1H-imidazol-5-yl)phenyl]-5,6-dihydro[1]benzoxepino[5,4-d]pyrimidin-4-amine (52 mg) as white crystals.

mp 235-237° C. Mass: 384 (m/z, (M+H)⁺) NMR(DMSO-d₆, δ): 2.36 (3H, s), 2.96 (2H, t, J=6.0 Hz), 3.59 (3H, s), 4.60 (2H, t, J=6.0 Hz), 6.89 (1H, s), 7.08-7.18 (2H, m), 7.28 (1H, ddd, J=7.7, 7.7, 1.3 Hz), 7.37-7.54 (2H, m), 7.66 (1H, d, J=7.7 Hz), 7.78 (1H, s), 7.90 (1H, dd, J=7.7, 1.7 Hz), 8.61 (1H, s), 8.96 (1H, s).

### EXAMPLE 87

A mixture of N-[3-(imidazol-1-yl)phenyl]-N-[5-(pyrrol-1-yl)pyridin-2-yl]-formamide (50 mg), methanol (5 ml) and a 1N solution of sodium hydroxide (1.5 ml) was heated under reflux for 8 hours. After cooling, the reaction mixture was partitioned between chloroform and water. The separated organic layer was dried over sodium sulfate, filtered and evaporated. The obtained residue was recrystallized from diethyl ether to give N-[3-(imidazol-1-yl)phenyl]-N-[5-(pyrrol-1-yl)pyridin-2-yl]-amine (30 mg).

mp: 152-156° C. IR (KBr, cm⁻¹): 1606, 1506 Mass: 302 (m/z, (M+H)⁺) NMR(DMSO-d₆, δ): 6.26 (2H, s), 6.97 (1H, d, J=9 Hz), 7.13 (2H, br s), 7.20-7.50 (3H, m), 7.50-7.70 (2H, m), 7.87 (1H, dd, J=9, 2 Hz), 8.07 (1H, br s), 8.16 (1H, br s), 8.48 (1H, s), 9.43 (1H, s).

### EXAMPLE 88

A solution of 5-chloro-N-(6-fluorobenzo[d]isoxazol-3-yl)benzene-1,3-diamine (0.15 g) and thiophene-2-carboximidothioic acid methyl ester hydroiodide (0.20 g) in 2-propanol (3 ml) was heated for 4 hours at 90° C. After cooling to ambient temperature, the resultant precipitate was collected by filtration, which was dissolved in dichloromethane. The solution was washed with an aqueous solution of sodium hydroxide and dried over potassium carbonate. After evaporation of the solvent, the residue was crystallized from methanol to give N-[3-chloro-5-[(6-fluorobenzo[d]isoxazol-3-yl)amino]phenyl]thiophene-2-carboxamidine (45.5 mg).

APCI-mass: 387 (m/z, (M+H)⁺) NMR(DMSO-d₆, δ): 6.50(1H, s), 6.67(2H, brs), 7.03-7.17(2H, m), 7.30(1H, dt, J=2.1, 9.0 Hz), 7.42(1H, d, J=1.9 Hz), 7.53-7.70(2H, m), 7.77(1H, d, J=3.3 Hz), 8.15(1H, dd, J=5.3, 8.7 Hz), 9.71(1H, s).

### EXAMPLE 89

A solution of 5-chloro-N-(6-fluorobenzo[d]isoxazol-3-yl)benzene-1,3-diamine (0.15 g) and methyl thiobenzimidate hydroiodide (0.24 g) in 2-propanol (3 ml) was heated for 4 hours at 90° C. After cooling to ambient temperature, the reaction mixture was diluted with dichloromethane and an aqueous solution of sodium hydroxide. The separated organic layer was dried over magnesium sulfate and evaporated under reduced pressure. The residue was crystallized from methanol to give N-[3-chloro-5-[(6-fluoro-benzo[d]isoxazol-3-yl)amino]phenyl]benzamidine (129 mg).

APCI-mass: 381 (m/z, (M+H)⁺) NMR(DMSO-d₆, δ): 6.30-6.70(3H, m), 7.09(1H, s), 7.20-7.50(5H, m), 7.60(1H, dd, J=2.1, 9.0 Hz), 7.80-8.08(2H, m), 8.15(1H, dd, J=5.3, 8.7 Hz), 9.71(1H, s).

### EXAMPLE 90

A mixture of 1-chloro-5-(thiophen-3-yl)isoquinoline (85 mg) and 3-(4,5-dimethylimidazol-1-yl)phenylamine (130 mg) was heated for 75 minutes at 190° C. After cooling to ambient temperature, the reaction mixture was taken up into a mixture of dichloromethane and a 4N aqueous solution of sodium hydroxide. The separated organic layer was washed with brine and dried over potassium carbonate. After evaporation under reduced pressure, the residue was chromatographed on silica gel eluting with 0%-2% methanol in dichloromethane. The obtained product was crystallized from methanol to give [3-(4,5-dimethylimidazol-1-yl)phenyl]-[5-(thiophen-3-yl)isoquinolin-1-yl]amine (29.2 mg).

APCI-mass; 397 (m/z, [M+H]⁺) NMR(DMSO-d₆, δ: 2.13(3H, s), 2.15(3H, s), 6.99(1H, d, J=9.1 Hz), 7.24(1H, d, J=6.0 Hz), 7.35(1H, dd, J=1.4,4.7 Hz), 7.47(1H, t, J=8.0 Hz), 7.59-7.83(5H, m), 7.94(1H, d, J=8.0 Hz), 7.98-8.10(2H, m), 8.56(1H, d, J=6.3 Hz), 9.45(1H, s).

### EXAMPLE 91

A mixture of 1-chloro-5-(thiophen-3-yl)isoquinoline (276 mg) and 3-(4-methylimidazol-1-yl)phenylamine (389 mg) was heated for 50 minutes at 190° C. After cooling to ambient temperature, the reaction mixture was taken up into a mixture of dichloromethane and a 10% aqueous potassium carbonate solution. The separated organic layer was washed with brine and dried over potassium carbonate. After evaporation of the solvent under reduced pressure, the residue was chromatographed on silica gel eluting with 0%-2% methanol in dichloromethane to give [[3-(4-methylimidazol-1-yl)phenyl]-[5-(thiophen-3-yl)isoquinolin-1-yl]amine (100.5 mg).

APCI-mass: 383 (m/z, [M+H]⁺) NMR(DMSO-d₆, δ): 2.18(3H, s), 7.10-7.29(2H,m), 7.29-7.51(3H, m), 7.60-7.81(4H, m), 7.86(1H, d, J=9.1 Hz), 7.99-8.13(2H, m), 8.16(1H, s), 8.55(1H, d, J=7.6 Hz), 9.42(1H, s).

### EXAMPLE 92

A mixture of 3-(4,5-dimethylimidazol-1-yl)phenylamine (100 mg) and 2,6-dichlorobenzimidazole (218 mg) was heated for 15 minutes at 190° C. After cooling to ambient temperature, the reaction mixture was dissolved in a small amount of methanol and diluted with dichloromethane. The mixture was washed in turn with a 1N aqueous solution of sodium hydroxide and brine, dried over potassium carbonate and evaporated under reduced pressure. The residue was chromatographed on silica gel eluting with a mixture of dichloromethane and methanol (0-3.5% V/V) to give (6-chlorobenzothiazol-2-yl)-[3-(4,5-dimethylimidazol-1-yl)phenyl]amine (10.8 mg).

APCI-mass: 355.27 (m/z, (M+H)⁺) NMR(DMSO-d₆, δ): 2.13(3H, s), 2.15(3H, s), 7.06(1H, d, J=8.7 Hz), 7.35(1H, dd, J=2.2, 8.7 Hz), 7.41-7.75(4H, m), 7.90-8.03(2H, m), 10.83(1H, s).

### EXAMPLE 93

A solution of 5-chloro-N-(6-chlorobenzothiazol-2-yl)-benzene-1,3-diamine (100 mg) and methyl thiobenzimidate hydroiodide (135 mg) in 2-propanol (2 ml) was heated for 4 hours at 90° C. After cooling to ambient temperature, the reaction mixture was diluted with dichloromethane and an aqueous solution of sodium hydroxide. The separated organic layer was dried over magnesium sulfate and evaporated under reduced pressure. The residue was chromatographed on silica gel eluting with a mixture of dichloromethane and methanol (0-3% V/V) to give N-[3-chloro-5-[(6-chlorobenzothiazol-2-yl)amino]phenyl]benzamidine (61.3 mg).

APCI-mass: 413.27, 415 (m/z, (M+H)⁺)s NMR(DMSO-d₆, δ): 6.40-6.70(2H, m), 7.08(1H, brs), 7.28-7.70(7H, m), 7.80-8.08(3H, m), 10.64(1H, s).

### EXAMPLE 94

A suspension of 8-(trifluoromethyl)-4(3H)-quinazolinone(118 mg) in phosphorus oxychloride (1 ml) was stirred for 6 hours at 130° C. and evaporated. To the residue was added 3-(1,2-dimethyl-1H-imidazol-5-yl)aniline (93 mg) and 1,3-dimethyl-2-imidazolidinone (1.5 ml), and the mixture was stirred for an hour at 130° C. After cooling to room temperature, the mixture was diluted with 1N-hydrochloric acid (50 ml) and washed with ethyl acetate (50 ml). The aqueous layer was separated and adjusted to pH 9.0 with a 4N aqueous solution of sodium hydroxide and extracted with ethyl acetate. The organic layer was washed with a 0.5N aqueous solution of sodium hydroxide and brine, dried over magnesium sulfate and evaporated. The residue was triturated with methanol, collected by filtration and washed with methanol and diisopropyl ether. The mixture was dried and evaporated to give N-[3-(1,2-dimethyl-1H-imidazol-5-yl)phenyl]-8-(trifluoromethyl)-4-quinazolinamine (121 mg).

ESI-Mass; 384.3 (m/z, (M+H)⁺) NMR(DMSO-d₆, δ): 2.37(3H, s), 3.73(3H, s), 6.92(1H, s), 7.23(1H, d, J=7.7 Hz), 7.50(1H, t, J=7.9 Hz), 7.7-7.9(2H, m), 7.93(1H, s), 8.28(1H, d, J=7.3 Hz), 8.72(1H, s), 8.86(1H, d, J=8.3 Hz), 10.14(1H, s).

### EXAMPLE 95

A suspension of 8-(trifluoromethyl)-4(3H)-quinazolinone (118 mg) in phosphorus oxychloride (1 ml) was stirred for 6 hours at 130° C. and evaporated. To the residue was added 3-(4,5-dimethyl-1H-imidazol-1-yl)aniline (93 mg) and 1,3-dimethyl-2-imidazolidinone (1.5 ml), and the mixture was stirred for an hour at 130° C. After cooling to room temperature, the mixture was diluted with 1N-hydrochloric acid (50 ml) and washed with ethyl acetate (50 ml). The separated aqueous layer was adjusted to pH 9.0 with a 4N aqueous solution of sodium hydroxide and extracted with ethyl acetate. The organic layer was washed with a 0.5N aqueous solution of sodium hydroxide and brine, dried over magnesium sulfate and evaporated. The residue was triturated with methanol, collected by filtration, washed with methanol and diisopropyl ether and dried to give N-[3-(4,5-dimethyl-1H-imidazol-1-yl)phenyl]-8-(trifluoromethyl)-4-quinazolinamine (113 mg).

ESI-Mass; 384.3 (m/z, (M+H)⁺) NMR(DMSO-d₆, δ): 2.13(3H, s), 2.16(3H, s), 7.20(1H, d, J=8.8 Hz), 7.56(1H, s, J=8.0 Hz), 7.69(1H, s), 7.81(1H, t, J=8.1 Hz), 7.92(1H, d, J=8.2 Hz), 8.02(1H, s), 8.29(1H, d, J=7.4 Hz), 8.76(1H, s), 8.86(1H, d, J=8.3 Hz), 10.20(1H, s).

### EXAMPLE 96

A suspension of 7-(trifluoromethyl)-4(3H)-quinazolinone (118 mg) in phosphorus oxychloride (1 ml) was stirred for 6 hours at 130° C. and evaporated. To the residue was added 3-(1,2-dimethyl-1H-imidazol-5-yl)aniline (93 mg) and 1,3-dimethyl-2-imidazolidinone (1.5 ml), and the mixture was stirred for an hour at 130° C. After cooling to room temperature, the mixture was diluted with 1N-hydrochloric acid (50 ml) and washed with ethyl acetate (50 ml). The separated aqueous layer was adjusted to pH 9.0 with a 4N aqueous solution of sodium hydroxide and extracted with ethyl acetate. The organic layer was washed with a 0.5N aqueous solution of sodium hydroxide and brine, dried over magnesium sulfate and evaporated. The residue was triturated with methanol, collected by filtration, washed with methanol and diisopropyl ether and dried to give N-[3-(1,2-dimethyl-1H-imidazol-5-yl)phenyl]-7-(trifluoromethyl)-4-quinazolinamine (93 mg).

APCI-mass: 384.20 (m/z, (M+H)⁺) NMR(DMSO-d₆, δ): 2.37(3H, s), 3.61(3H, s), 6.92(1H, s), 7.22(1H, d, J=7.9 Hz), 7.49(1H, t, J=7.9 Hz), 7.85(1H, d, J=8.1 Hz), 7.9-8.1(2H, m), 8.13(1H, s), 8.72(1H, s), 8.82(1H, d, J=8.7 Hz), 10.17(1H, s).

### EXAMPLE 97

A suspension of 7-(trifluoromethyl)-4(3H)-quinazolinone (118 mg) in phosphorus oxychloride (1 ml) was stirred for 6 hours at 130° C. and evaporated. To the residue was added 3-(4.5-dimethyl-1H-imidazol-1-yl)aniline (93 mg) and 1,3-dimethyl-2-imidazolidinone (1.5 ml), and the mixture was stirred for an hour at 130° C. After cooling to room temperature, the mixture was diluted with 1N-hydrochloric acid (50 ml) and washed with ethyl acetate (50 ml). The separated aqueous layer was adjusted to pH 9.0 with a 4N aqueous solution of sodium hydroxide and extracted with ethyl acetate. The organic layer was washed with a 0.5N aqueous solution of sodium hydroxide and brine, dried over magnesium sulfate and evaporated. The residue was triturated with methanol, collected by filtration, washed with methanol and diisopropyl ether and dried to give N-[3-(4,5-dimethyl-1H-imidazol-1-yl)phenyl]-7-(trifluoromethyl)-4-quinazolinamine (67 mg).

APCI-mass: 384.13 ((m/z, M+H)⁺) NMR(DMSO-d₆, δ): 2.13(3H, s), 2.16(3H, s), 7.20(1H, d, J=7.9 Hz), 7.57(1H, t, J=8.1 Hz), 7.69(1H, s), 7.9-8.1(3H, m), 8.14(1H, s), 8.75(1H, s), 8.82(1H, d, J=8.7 Hz), 10.24(1H, s).

### EXAMPLE 98

A suspension of 8-(thiophen-2-yl)-4(3H)-quinazolinone (92 mg) in phosphorus oxychloride (1 ml) was stirred for 6 hours at 130° C. and evaporated. To the residue was added 5-amino-2-[(pyridin-2-yl)methylamino]pyrimidine (81 mg) and 1,3-dimethyl-2-imidazolidinone (2 ml), and the mixture was stirred for an hour at 130° C. After cooling to room temperature, the mixture was diluted with 1N-hydrochloric acid (50 ml) and washed with ethyl acetate (50 ml). The separated aqueous layer was adjusted to pH 9.0 with a 4N aqueous solution of sodium hydroxide and extracted with ethyl acetate. The organic layer was washed with a 0.5N aqueous solution of sodium hydroxide and brine, dried over magnesium sulfate and evaporated. The residue was crystallized form diisopropyl ether and methanol, collected by filtration, washed with methanol and diisopropyl ether and dried to give N-[2-[(pyridin-2-yl)methylamino]pyrimidin-5-yl]-8-(thiophen-2-yl)-4-quinazolinamine (65 mg).

APCI-MASS: 412.07 (m/z, (M+H)⁺) NMR(DMSO-d₆, δ): 4.63(2H, d, J=6.2 Hz), 7.1-7.3(2H, m), 7.33(1H, d, J=7.9 Hz), 7.6-8.0(5H, m), 8.3-8.5(3H, m), 8.57(2H, s), 8.60(1H, s), 9.79(1H, s).

### EXAMPLE 99

A mixture of methyl benzenecarbimidothioate hydroiodide (279 mg), N¹-(1,2-benzo[d]isoxazol-3-yl)-5-chloro-1,3-benzenediamine (130 mg) and methanol (2 ml) was heated under reflux for three hours. After cooling to room temperature, dichloromethane (50 ml), water (50 ml) and a 1N aqueous solution of sodium hydroxide (2 ml) were added to the mixture and the organic phase was extracted with dichloromethane (20 ml, twice). The combined organic phases were dried over magnesium sulfate, filtered and evaporated. The residue was purified by a silica gel column chromatography eluting with a mixture of dichloromethane, methanol and ammonia. The obtained product was recrystallized from methanol to give N-[3-(1,2-benzo[d]isoxazol-3-ylamino)-5-chlorophenyl]benzenecarboximidamide (36 mg) as white crystals.

mp 190-191° C. Mass: 363 (m/z, (M+H)⁺) NMR(DMSO-d₆, δ): 6.40-6.61 (3H, m), 7.12 (1H, s), 7.33-7.58 (5H, m), 7.59-7.74 (2H, m), 7.83-8.05 (2H, m), 8.13 (1H, d, J=7.7 Hz), 9.65 (1H, s).

### EXAMPLE 100

The following compounds described in (1) and (2) were obtained in a manner similar to Example 99.

(1) N-[3-(1,2-Benzo[d]isoxazol-3-ylamino)-5-chlorophenyl]-2-thiophenecarboximidamide

mp 201-202° C. Mass: 369 (m/z, (M+H)⁺) NMR(DMSO-d₆, δ): 6.49 (1H, s), 6.66 (2H, s); 7.07-7.16 (2H, m), 7.35-7.44 (1H, m), 7.46-7.49 (1H, m), 7.59-7.70 (3H, m), 7.78 (1H, d, J=3.4 Hz), 8.13 (1H, d, J=8.0 Hz), 9.66 (1H, s).

(2) N-[3-(1,2-Benzo[d]isoxazol-3-ylamino)-5-(trifluoromethyl)phenyl]-2-thiophenecarboximidamide

mp 211-212° C. Mass: 403 (m/z, (M+H)⁺) NMR(DMSO-d₆, δ): 6.15 (3H, br s), 7.10-7.18 (1H, m), 7.36-7.46 (1H, m), 7.48 (1H, s), 7.59-7.68 (3H, m), 7.72 (1H, s), 7.79 (1H, d, J=3.0 Hz), 8.14 (1H, d, J=7.6 Hz), 9.82 (1H, s).

### EXAMPLE 101

To a solution of 3-(1,2-dimethyl-1H-imidazol-5-yl)aniline (187 mg) in tetrahydrofuran (5 ml) was added a 1.5M solution of n-butyl lithium in n-hexane (0.71 ml) dropwise with stirring at 0° C. followed by stirring for additional 30 minutes at the temperature. To the reaction mixture was added 3-chloro-1,2-benzo[d]isoxazole (184 mg), and the stirring was continued for 63 hours at ambient temperature. The reaction mixture was diluted with ethyl acetate (30 ml) and washed with water (3×30 ml), an aqueous saturated solution of ammonium chloride (30 ml×2), an aqueous saturated solution of sodium hydrogencarbonate (30 ml) and brine (20 ml). The organic layer was dried over magnesium sulfate and filtered. After evaporation of the solvent, the residue was chromatographed on silica gel eluting with a mixture of dichloromethane and methanol to give N-[3-(1,2-dimethyl-1H-imidazol-5-yl)phenyl]-1,2-benzo[d]isoxazol-3-amine (37 mg).

Mass: 305 (m/z, (M+H)⁺) NMR(DMSO-d₆, δ): 2.38(3H, s), 3.59(3H, s), 6.90(1H, s), 7.04(1H, d, J=7.0 Hz), 7.3-7.8(5H, m), 7.80(1H, brs), 8.17(1H, d, J=7.7 Hz), 9.69(1H, brs).

### EXAMPLE 102

To a solution of 3-bromo-N-[3-(1,2-dimethyl-1H-imidazol-5-yl)phenyl]-2-fluoro-N′-hydroxybenzenecarboximidamide (560 mg) in N-methyl-2-pyrrolidone (20 ml) was added potassium tert-butoxide (156 mg) under stirring at 0° C. After stirring for 10 minutes at 0° C., the reaction mixture was heated for 2 hours at 100° C. After cooling, the reaction mixture was diluted with water (100 ml) and extracted with ethyl acetate (100 ml×2). The combined extracts were washed with an aqueous saturated solution of ammonium chloride (100 ml×2), an aqueous saturated solution of sodium hydrogencarbonate (100 ml) and brine (100 ml). The organic layer was dried over magnesium sulfate and filtered. After evaporation of the solvent, the residue was triturated with ethyl acetate to give 7-bromo-N-[3-(1,2-dimethyl-1H-imidazol-5-yl)phenyl]-1,2-benzo[d]isoxazol-3-amine (393 mg) as crystals.

Mass: 383,385 (1:1 ratio, Br isotopes, m/z, (M+H)⁺) NMR(DMSO-d₆, δ) 2.38 (3H, s), 3.59 (3H, s), 6.90 (1H, s), 7.07 (1H, d, J=7.7 Hz), 7.36 (1H, t, J=7.8 Hz), 7.47 (1H, t, J=7.9 Hz), 7.6-7.7 (1H, m), 7.7-7.9 (1H, m), 7.90 (1H, d, J=7.1 Hz), 8.18 (1H, d, J=7.3 Hz), 9.79 (1H, br s).

### EXAMPLE 103

The following compound described in (1) and (2) were obtained in a manner similar to Example 102.

(1) N-[3-(1,2-dimethyl-1H-imidazol-5-yl)phenyl]-7-(2-thienyl)-1,2-benzo[d]isoxazol-3-amine

Mass: 387 (m/z. (M+H)⁺) NMR(DMSO-d₆, δ): 2.38 (3H, s), 3.60 (3H, s), 6.91 (1H, s), 7.06 (1H, d, J=7.7 Hz), 7.2-7.4 (1H, m), 7.4-7.6 (2H, m), 7.6-7.8 (2H, m), 7.8-7.9 (2H, m), 7.97 (1H, d, J=7.4 Hz), 8.11 (1H, d, J=7.2 Hz), 9.75 (1H, br s).

(2) N-[3-(1,2-dimethyl-1H-imidazol-5-yl)phenyl]-7-(3-thienyl)-1,2-benzo[d]isoxazol-3-amine

Mass: 387 (m/z, (M+H)⁺) NMR(DMSO-d₆, δ): 2.38 (3H, s), 3.60 (3H, s), 6.91 (1H, s), 7.06 (1H, d, J=7.6 Hz), 7.3-8.3 (9H, m), 9.72 (1H, br s).

### EXAMPLE 104

To a mixture of 7-bromo-N-[3-(1,2-dimethyl-1H-imidazol-5-yl)phenyl]-1,2-benzo[d]isoxazol-3-amine (100 mg), 4-fluorophenylboronic acid (47 mg) and 1,2-dimethoxyethane (1 ml) were added a 2M aqueous solution of sodium carbonate (0.43 ml) and tetrakis (triphenylphosphine)palladium(0) (15 mg) at ambient temperature. The mixture was heated for 89 hours at 90° C. After cooling, the reaction mixture was diluted with ethyl acetate (50 ml) and washed with water (50 ml) and brine (50 ml×3). The organic layer was dried over potassium carbonate and filtered. After evaporation of the solvent, the residue was chromatographed on silica gel eluting with a mixture of dichloromethane and methanol and triturated with ethyl acetate to give N-[3-(1,2-dimethyl-1H-imidazol-5-yl)phenyl]-7-(4-fluorophenyl)-1,2-benzo[d]isoxazol-3-amine (30 mg) as crystals.

Mass: 399 (m/z, (M+H)⁺) NMR(DMSO-d₆, δ): 2.38 (3H, s), 3.60 (3H, s), 6.90 (1H, s), 7.06 (1H, d, J=7.7 Hz), 7.3-7.6 (4H, m), 7.6-7.8 (1H, m), 7.8-8.1 (4H, m), 8.17 (1H, d, J=7.2 Hz), 9.73 (1H, br s).

### EXAMPLE 105

To a suspension of N-[3-(1,2-dimethyl-1H-imidazol-5-yl)phenyl]-N′-(2-pyridylmethyl)thiourea (337 mg) in toluene (10 ml) was added 1-ethyl-3-(3-dimethylaminopropyl)carbodiimide hydrochloride (288 mg) at ambient temperature, and the mixture was heated for 45 minutes at 110° C. After cooling, the reaction mixture was diluted with ethyl acetate (20 ml), washed with an aqueous sodium hydrogencarbonate solution (30 ml), water (30 ml) and brine (30 ml). The organic layer was dried over magnesium sulfate and filtered. After evaporation of the solvent, the residue was chromatographed on silica gel eluting with a mixture of dichloromethane, methanol and ethyl acetate (25:1:1) to give N-[3-(1,2-dimethyl-1H-imidazol-5-yl)phenyl]imidazo[1,5-a]pyridin-3-amine (255 mg).

Mass: 304 (m/z, (M+H)⁺) NMR(DMSO-d₆, δ): 2.34 (3H, s), 3.51 (3H, s), 6.5-6.7 (2H, m), 6.80 (1H, s), 6.86 (1H, d, J=7.3 Hz), 7.1-7.5 (5H, m), 8.00 (1H, d, J=6.5 Hz), 8.86 (1H, br s).

### EXAMPLE 106

8-Chloro-N-[3-(1,2-dimethyl-1H-imidazol-5-yl)phenyl]-6-(trifluoromethyl)imidazo [1,5-a]pyridin-3-amine was obtained in a manner similar to Example 105.

Mass: 406 (m/z, (M+H)⁺) NMR(DMSO-d₆, δ): 2.36 (3H, s), 3.55 (3H, s), 6.85 (1H, s), 6.9-7.1 (2H, m), 7.3-7.5 (2H, m), 7.5-7.8 (2H, m), 8.83 (1H, br s), 9.40 (1H, br s).

### EXAMPLE 107

The mixture of N¹-(imidazo[1,5-a]pyridin-3-yl)-1,3-benzenediamine (120 mg), methyl benzenecarbimidothioate hydroiodide (299 mg), and methanol (2 ml was heated under reflux for 3 hours. After cooling, the reaction mixture was poured into a 0.1N aqueous solution of sodium hydroxide (55 ml) and the resulting mixture was extracted with dichloromethane (50 ml, 20 ml×2). The combined organic extracts were dried over magnesium sulfate and filtered. After evaporation of the solvent, the residue was chromatographed on silica gel eluting with a mixture of dichloromethane, methanol and a 28% aqueous solution of ammonium hydroxide in water (250:10:1) to give N-[3-(imidazo[1,5-a]pyridin-3-ylamino)phenyl]benzenecarboximidamide (135 mg) as crystals.

Mass: 328 (m/z, (M+H)⁺) NMR(DMSO-d₆, δ): 6.22 (2H, br s), 6.33 (1H, d, J=7.5 Hz), 6.4-6.9 (4H, m), 7.0-7.3 (2H, m), 7.3-7.6 (4H, m), 7.8-8.1 (3H, m), 8.62 (1H, br s).

### EXAMPLE 108

3-Bromo-N-[3-(1,2-dimethyl-1H-imidazol-5-yl)phenyl]-2-fluorobenzenecarbohydrazonamide (68 mg) was heated for 30 minutes at 220° C. After cooling, the resultant solid was partitioned between an aqueous sodium hydrogencarbonate solution (20 ml) and ethyl acetate (20 ml). The organic layer was washed with water (20 ml) and brine (20 ml), dried over potassium carbonate, and filtered. After evaporation of the solvent, the residue was chromatographed on silica gel eluting with a mixture of dichloromethane and methanol. The obtained product was triturated with ethyl acetate to give 7-bromo-N-[3-(1,2-dimethyl-1H-imidazol-5-yl)phenyl]-1H-indazol-3-amine (25 mg) as crystals.

Mass: 382, 384 (1:1 ratio, Br isotopes, m/z, (M+H)⁺) NMR(DMSO-d₆, δ): 2.36 (3H, s), 3.56 (3H, s), 6.7-7.0 (2H, m), 7.00 (1H, t, J=7.7 Hz), 7.2-7.5 (1H, m), 7.5-7.8 (2H, m), 7.81 (1H, br s), 8.01 (1H, d, J=8.0 Hz), 9.09 (1H, br s), 12.43 (1H, br s).

### EXAMPLE 109

To a mixture of 8-(2-thienyl)-4-quinazolinol (110 mg) and phosphorous oxychloride (1.5 ml) was added a small amount of N,N-dimethylformamide. The mixture was refluxed under nitrogen atmosphere for 2.5 hours at 100° C. and concentrated in vacuo to give crude 4-chloro-8-(2-thienyl)quinazoline. To a suspension of crude 4-chloro-8-(2-thienyl)quinazoline in 1,3-dimethyl-2-imidazolidinone (1.5 ml) was added 3-(4-methyl-1H-imidazol-1-yl)aniline (83.5 mg). The mixture was stirred under nitrogen atmosphere for 1.5 hours at 120° C. To the mixture was added an aqueous saturated solution of sodium hydrogencarbonate and extracted with ethyl acetate (20 ml×3). The combined extracts were washed with water and brine, then dried over magnesium sulfate and evaporated under reduced pressure. The residue was purified by a silica gel column chromatography eluting with a 1/20 mixture of methanol/dichloromethane to give N-[3-(4-methyl-1H-imidazol-1-yl)phenyl]-8-(2-thienyl)-4-quinazolinamine (56 mg, 30.3%).

APCI-mass: 384 (m/z, (M+H)⁺) NMR(DMSO-d₆, δ): 2.18(3H, s), 7.18(1H, dd, J=5.1, 3.7 Hz), 7.38-7.42(2H, m), 7.52(1H, t, J=7.6 Hz), 7.66-7.94(3H, m), 8.12(2H, dd, J=6.2, 1.7 Hz), 8.37(1H, d, J=7.6 Hz), 8.51(1H, d, J=7.6 Hz), 8.76(1H, s), 10.01(1H, s).

### EXAMPLE 110

To a mixture of 8-(2-thienyl)-4-quinazolinol (170 mg) and phosphorous oxychloride (1.7 ml) was added small amount of N,N-dimethylformamide. The mixture was refluxed under nitrogen atmosphere for 2.5 hours at 100° C. and concentrated in vacuo to give crude 4-chloro-8-(2-thienyl)quinazoline. To a suspension of crude 4-chloro-8-(2-thienyl)quinazoline in 1,3-dimethyl-2-imidizolidinone (2.5 ml) was added 3-(1,2-dimethyl-1H-imidazol-5-yl)aniline (126 mg). The mixture was stirred under nitrogen atmosphere for 1.5 hours at 120° C. To the mixture was added an aqueous saturated solution of sodium hydrogencarbonate and the resulting mixture was extracted with ethyl acetate (20 ml×3). The combined extracts were washed with water and brine, then dried over magnesium sulfate and evaporated under reduced pressure. The residue was purified by a silica gel column chromatography eluting with 6/3/100 mixture of methanol/ethyl acetate/dichloromethane to give N-[3-(1,2-dimethyl-1H-imidazol-5-yl)phenyl]-8-(2-thienyl)-4-quinazolinamine (124 mg, 41.6%).

APCI-mass: 398 (m/z, (M+H)⁺) NMR(DMSO-d₆, δ): 2.37(3H, s), 3.61(3H, s), 7.15(1H, s), 7.17-7.23(2H, m), 7.49(1H, t, J=7.8 Hz), 7.65-7.73(2H, m), 7.86(1H, d, J=8.5 Hz), 7.91-7.96(2H, m), 8.37(1H, d, J=7.6 Hz), 8.49(1H, d, J=7.6 Hz), 8.72(1H, s), 9.95(1H, s).

### EXAMPLE 111

To a mixture of 8-(2-thienyl)-4-quinazolinol (150 mg) and phosphorous oxychloride (1.7 ml) was added a small amount of N,N-dimethylformamide. The mixture was refluxed under nitrogen atmosphere for 2.5 hours and concentrated in vacuo to give crude 4-chloro-8-(2-thienyl)quinazoline. To a suspension of crude 4-chloro-8-(2-thienyl)quinazoline in 1,3-dimethyl-2-imidazolidinone (1.5 ml) was added 3-(4,5-dimethyl-1H-imidazol-1-yl)aniline(117 mg). The mixture was stirred under nitrogen atmosphere for 1.5 hours at 120° C. To the mixture was added an aqueous saturated solution of sodium hydrogencarbonate and extracted with ethyl acetate (20 ml×3). The combined extracts were washed with water and brine, then dried over magnesium sulfate and evaporated under reduced pressure. The residue was triturated with methanol, collected by filtration and dried to give N-[3-(4,5-dimethyl-1H-imidazol-1-yl)phenyl]-8-(2-thienyl)-4-quinazolinamine (100 mg, 38.3%).

APCI-mass: 398 (m/z, (M+H)⁺) NMR(DMSO-d₆, δ): 2.13(3H, s), 2.16(3H, s), 7.16-7.20(2H, m), 7.56(1H, t, J=8.0 Hz), 7.66-7.75(2H, m), 7.69(1H, s), 7.91-7.98(2H, m), 8.03-8.05(1H, m), 8.37(1H, d, J=7.5 Hz), 8.50(1H, d, J=7.5 Hz), 8.76(1H, s), 10.02(1H, s)

### EXAMPLE 112

To a mixture of 8-(2-thienyl)-4-quinazolinol (150 mg) and phosphorous oxychloride (1.5 ml) was added small amount of N,N-dimethylformamide. The mixture was refluxed under nitrogen atmosphere for 2 hours at 100° C. and concentrated in vacuo to give crude 4-chloro-8-(2-thienyl)quinazoline. To a suspension of crude 4-chloro-8-(2-thienyl)quinazoline in 1,3-dimethyl-2-imidazolidinone (1.5 ml) was added 3-(3-methyl-1H-1,2,4-triazol-1-yl)phenylamine (115 mg). The mixture was stirred under nitrogen atmosphere for 1.5 hours at 120° C. To the mixture was added an aqueous saturated solution of sodium hydrogencarbonate and extracted with ethyl acetate (20 ml×3). The combined extracts were washed with water and brine, then dried over magnesium sulfate and evaporated under reduced pressure. The residue was purified by a silica gel column chromatography eluting with 6/3/100 mixture of methanol/ethyl acetate/dichloromethane to give N-[3-(3-methyl-1H-1,2,4-triazol-1-yl)phenyl]-8-(2-thienyl)-4-quinazolinamine (86 mg, 39.6%).

APCI-mass: 385 (m/z, (M+H)⁺) NMR(DMSO-d₆, δ): 2.39(3H, s), 7.18(1H, dd, J=5.1, 3.8 Hz), 7.56-7.75(3H, m), 7.70(1H, d, J=2.8 Hz), 7.93(1H, dd, J=3.8, 1.1 Hz), 7.96-7.98(1H, m), 8.36(1H, s), 8.38(1H, d, J=7.6 Hz), 8.53(1H, d, J=7.6 Hz), 8.77(1H, s), 9.16(1H, s), 10.07(1H, s).

### EXAMPLE 113

To a suspension of 1-chloro-4-(4-fluorobenzyl)phthalazine (300 mg) in pyridine (5.0 ml) was added 3-(1,2-dimethyl-1H-imidazol-5-yl)aniline (411 mg). The mixture was refluxed under nitrogen atmosphere for 18 hours and evaporated under reduced pressure. The mixture was diluted with dichloromethane and washed with an aqueous saturated solution of sodium hydrogencarbonate and brine. The mixture was dried over sodium sulfate and evaporated under reduced pressure. The residue was purified by a silica gel column chromatography eluting with 3-10% methanol in dichloromethane to give N-[3-(1,2-dimethyl-1H-imidazol-5-yl)phenyl]-4-(4-fluorobenzyl)-1-pthalazinamine (73 mg, 15.6%).

APCI-mass: 424 (m/z, (M+H)⁺) NMR(DMSO-d₆, δ): 2.37(3H, s), 3.73(3H, s), 4.54(2H, s), 6.88(1H, s), 7.04-7.13(3H, m), 7.33-7.47(3H, m), 7.88-7.97(3H, m), 8.02(1H, d, J=1.7 Hz), 8.14(1H, dd, J=7.5, 1.7 Hz), 8.59(1H, d, J=7.5 Hz), 9.22(1H, s).

### EXAMPLE 114

To a suspension of 1-benzyl-4-chloropthalazine (300 mg) in pyridine (3.0 ml) was added 3-(1,2-dimethyl-1H-imidazol-5-yl)aniline (287 mg). The mixture was refluxed under nitrogen atmosphere for 24 hours and evaporated under reduced pressure. The mixture was diluted with dichloromethane and washed with an aqueous saturated solution of sodium hydrogencarbonate and brine. The mixture was then dried over sodium sulfate and evaporated under reduced pressure. The residue was purified by a silica gel column chromatography eluting with 2-8% methanol in dichloromethane to give 4-benzyl-N-[3-(1,2-dimethyl-1H-imidazol-5-yl)phenyl]-1-pthalazinamine (288 mg, 60.3%).

APCI-mass: 406 (m/z, (M+H)⁺) NMR(DMSO-d₆, δ): 2.37(3H, s), 3.59(3H, s), 4.55(2H, s), 6.88(1H, s), 7.07(1H, d, J=7.8 Hz), 7.12-7.35(5H, m), 7.43(1H, t, J=7.8 Hz), 7.86-8.00(3H, m), 8.03(1H, s), 8.13(1H, d, J=7.4 Hz), 8.59(1H, d, J=7.4 Hz), 9.21(1H, s).

### EXAMPLE 115

A mixture of 4-benzyl-1-chloroisoquinolne (200 mg) and 3-(1,2-dimethyl-1H-imidazol-5-yl)aniline (295 mg) was stirred under nitrogen atmosphere for 1.5 hours at 190° C. To the mixture was added dichloromethane (50 ml) and a 30 wt % aqueous solution of sodium hydroxide (30 ml) and stirred for 3 minutes. The aqueous layer was separated and extracted with dichloromethane (30 ml×2). The combined extracts were washed with brine, dried over sodium sulfate and evaporated under reduced pressure. The residue was purified by a silica gel column chromatography eluting with 2-5% methanol in dichloromethane to give 4-benzyl-N-[3-(1,2-dimethyl-1H-imidazol-5-yl)phenyl]-1-isoquinolinamine (95 mg, 30.0%).

APCI-mass: 405 (m/z, (M+H)⁺) NMR(DMSO-d₆, δ): 2.37(3H, s), 3.60(3H, s), 4.23(2H, s), 6.87(1H, s), 7.02(1H, d, J=7.7 Hz), 7.15-7.25(5H, m), 7.38(1H, t, J=7.7 Hz), 7.57-7.72(2H, m), 7.88(2H, d, J=8.0 Hz), 7.98(1H, s), 8.56(1H, d, J=7.7 Hz), 9.23(1H, s).

### EXAMPLE 116

To a suspension of 1,4-dichloropthalazine (700 mg) in pyridine (8.0 ml) was added 3-(1,2-dimethyl-1H-imidazol-5-yl)aniline (592 mg). The mixture was refluxed under nitrogen atmosphere for 24 hours and evaporated under reduced pressure. The mixture was diluted with dichloromethane and washed with an aqueous saturated solution of sodium hydrogencarbonate and brine. The mixture was then dried over sodium sulfate and evaporated under reduced pressure. The residue was purified by a silica gel column chromatography eluting with 3-10% methanol in dichloromethane to give 4-chloro-N-[3-(1,2-dimethyl-1H-imidazol-5-yl)phenyl]-1-pthalazinamine (359 mg, 27.3%).

APCI-mass: 350 (m/z, (M+H)⁺) NMR(DMSO-d₆, δ): 2.37(3H, s), 3.59(3H, s), 6.89(1H, s), 7.13(1H, d, J=7.8 Hz), 7.45(1H, t, J=7.8 Hz), 7.86(1H, d, J=8.2 Hz), 7.97(1H, s), 8.06-8.22(2H, m), 8.13(1H, d, J=7.4 Hz), 8.68(1H, d, J=8.2 Hz),9.45(1H, s).

### EXAMPLE 117

To a suspension of 4-chloro-N-[3-(1,2-dimethyl-1H-imidazol-5-yl)phenyl]-1-pthalazinamine (177 mg) in pyridine (2.5 ml) was added aniline (0.14 ml). The mixture was refluxed under nitrogen atmosphere for 10 hours and evaporated under reduced pressure. The mixture was diluted with dichloromethane (60 ml) and methanol (5.0 ml) and washed with an aqueous saturated solution of sodium hydrogencarbonate and brine. The mixture was then dried over sodium sulfate and evaporated under reduced pressure. The residue was purified by a silica gel column chromatography eluting with 5% methanol in dichloromethane to give N¹-[3-(1,2-dimethyl-1H-imidazol-5-yl)phenyl]-N⁴-phenyl-1,4-pthalazinediamine (81 mg, 40.0%).

APCI-mass: 407 (m/z, (M+H)⁺) NMR(DMSO-d₆, δ): 2.36(3H, s), 3.56(3H, s), 6.85(1H, s), 6.89-6.99(2H, m), 7.30-7.42(3H, m), 7.81-7.89(3H, m), 7.85(1H, s), 8.00-8.05(2H, m), 8.50-8.53(2H, m), 8.17(1H, s), 8.90(1H, s).

### EXAMPLE 118

To a suspension of 4-chloro-N-[3-(1,2-dimethyl-1H-imidazol-5-yl)phenyl]-1-pthalazinamine (150 mg) and sodium hydride (60% dispersion in mineral oil, 17 mg) in N,N-dimethylformamide (1.5 ml) was added phenol (40 mg). After hydrogen gas evolution has ceased, the mixture was stirred under nitrogen atmosphere for 48 hours at 120° C. and evaporated under reduced pressure. The mixture was diluted with methanol (5 ml) and dichloromethane (50 ml) and washed with water and brine. The mixture was then dried over magnesium sulfate and evaporated under reduced pressure. The residue was purified by a silica gel column chromatography eluting with 3-10% methanol in dichloromethane to give N-[3-(1,2-dimethyl-1H-imidazol-5-yl)phenyl]-4-phenoxy-1-pthalazinamine (89 mg, 51.0%).

APCI-mass: 408 (m/z, (M+H)⁺) NMR(DMSO-d₆, δ): 2.34(3H, s), 3.54(3H, s), 6.84(1H, s), 7.02(1H, d, J=7.5 Hz), 7.21-7.49(6H, m), 7.89(1H, s), 7.91(1H, d, J=8.0 Hz), 8.02-8.15(2H, m), 8.30(1H, d, J=8.0 Hz), 8.63(1H, d, J=7.5 Hz), 9.15(1H, s).

### EXAMPLE 119

A mixture of 1-chloro-4-(2-thienylmethyl)isoquinoline (280 mg) and 3-(1,2-dimethyl-1H-imidazol-5-yl)aniline (403 mg) was stirred under nitrogen atmosphere for 1.5 hours at 190° C. The mixture was diluted with dichloromethane (70 ml) and methanol (7 ml) and washed with water and brine. The mixture was then dried over sodium sulfate and evaporated under reduced pressure. The residue was purified by a silica gel column chromatography eluting with 2-5% methanol in dichloromethane to give N-[3-(1,2-dimethyl-1H-imidazol-5-yl)phenyl]-4-(2-thienylmethyl)-1-isoquinolinamine (166 mg, 37.7%).

APCI-mass: 411 (m/z, (M+H)⁺) NMR(DMSO-d₆, δ): 2.37(3H, s), 3.60(3H, s), 4.43(2H, s), 6.87(1H, s), 6.90-6.93(2H, m), 7.02(1H, d, J=7.8 Hz), 7.27(1H, dd, J=5.0, 1.5 Hz), 7.39(1H, t, J=7.8 Hz), 7.63-7.85(2H, m), 7.93-7.98(2H, m), 7.99(1H, s), 8.00(1H, s), 8.56(H, d, J=7.8 Hz), 9.25(1H, s).

### EXAMPLE 120

A mixture of 4-chloro-8-(3-thienyl)quinazoline (170 mg) and 3-(1,2-dimethyl-1H-imidazol-5-yl)aniline (142 mg) in 1,3-dimethyl-2-imidazolidinone (1.5 ml) was stirred under nitrogen atmosphere for 2.5 hours at 120° C. To the mixture was added an aqueous saturated solution of sodium hydrogencarbonate and extracted with ethyl acetate (20 ml×3). The combined extracts were washed with water and brine, then dried over magnesium sulfate and evaporated under reduced pressure. The residue was purified by a silica gel column chromatography eluting with 6/3/100 mixture of methanol/ethyl acetate/dichloromethane to give N-[3-(1,2-dimethyl-1H-imidazol-5-yl)phenyl]-8-(3-thienyl)-4-quinazolinamine (170 mg, 62.1%).

APCI-mass: 398 (m/z, (M+H)⁺) NMR(DMSO-d₆, δ): 2.37(3H, s), 3.60(3H, s), 6.91(1H, s), 7.19(1H, d, J=7.8 Hz), 7.48(1H, t, J=7.8 Hz), 7.59-7.65(1H, m), 7.69-7.74(2H, m), 7.88(1H, d, J=8.1 Hz), 7.97(1H, s), 8.09(1H, d, J=7.3 Hz), 8.16-8.18(1H, m), 8.53(1H, d, J=8.1 Hz), 8.67(1H, s), 9.91(1H, s).

### EXAMPLE 121

A mixture of 4-chloro-8-(3-thienyl)quinazoline (72 mg) and 3-(4,5-dimethyl-1H-imidazol-1-yl)aniline (50 mg) in 1,3-dimethyl-2-imidazolidinone (0.8 ml) was stirred under nitrogen atmosphere for 2.5 hours at 120° C. To the mixture was added an aqueous saturated solution of sodium hydrogencarbonate and extracted with ethyl acetate (15 ml×3). The combined ex%tracts were washed with water and brine, then dried over magnesium sulfate and evaporated under reduced pressure. The residue was purified by a silica gel column chromatography eluting with 6/3/100 mixture of methanol/ethyl acetate/dichloromethane to give N-[3-(4,5-dimethyl-1H-imidazol-1-yl)phenyl]-8-(3-thienyl)-4-quinazolinamine (84 mg, 83.1%).

APCI-mass: 398 (m/z, (M+H)⁺) NMR(DMSO-d₆, δ): 2.13(3H, s), 2.16(3H, s), 7.15(1H, d, J=8.1 Hz), 7.54(1H, t, J=8.1 Hz), 7.60-7.64(1H, m), 7.68(1H, s), 7.70-7.74(2H, m), 7.96(1H, d, J=8.1 Hz), 8.06(1H, d, J=7.3 Hz), 8.16-8.19(1H, m), 8.53(1H, d, J=7.3 Hz), 8.70(1H, s), 10.00(1H, s).

### EXAMPLE 122

A mixture of 9-fluoro-5,6-dihydrobenzo[h]quinazolin-4-ol (216 mg), phosphorous oxychloride (766 mg) and toluene (5 ml) was heated for 3 hours at reflux. After cooling, the reaction mixture was evaporated and the resultant residue was taken up into 1,3-dimethyl-2-imidazolidinone (2 ml) to give a solution of crude 4-chloro-9-fluoro-5,6-dihydrobenzo[h]quinazoline. To the solution was added 3-(4,5-dimethyl-1H-imidazol-1-yl)aniline (187 mg) and the mixture was stirred for 14 hours at 180° C. After cooling, the reaction mixture was dissolved in 1N-hydrochloric acid (50 ml), washed with dichloromethane (30 ml×2), neutralized with 30% aqueous solution of sodium hydroxide, and then extracted with dichloromethane (30 ml×4). The combined organic extracts were dried over magnesium sulfate, decolorized by activated charcoal and then filtered through Celite. After evaporation of the solvent, the residue was triturated with ethyl acetate to give N-[3-(4,5-dimethyl-1H-imidazol-1-yl)phenyl]-9-fluoro-5,6-dihydrobenzo[h]quinazolin-4-amine (123 mg) as white crystals.

Mass: 386 (m/z. (M+H)⁺) NMR(DMSO-d₆, δ): 2.14 (6H, br s), 2.96 (4H, br s), 7.0-7.6 (4H, m), 7.73 (1H, s), 7.7-8.0 (3H, m), 8.62 (1H, s), 8.91 (1H, br s).

### EXAMPLE 123

The following compounds described in (1) to (4) were obtained in a manner similar to Example 122.

(1) 9-Fluoro-N-[3-(4-methyl-1H-imidazol-1-yl)phenyl]-5,6-dihydrobenzo[h]quinazolin-4-amine, white crystals.

Mass: 372 (m/z. (M+H)⁺) NMR(DMSO-d₆, δ): 2.18 (3H, s), 2.8-3.1 (4H, m), 7.1-7.6 (5H, m), 7.6-7.8(1H, m), 7.88 (1H, dd, J=2.7, 10.1 Hz), 7.9-8.1 (2H, m), 8.63 (1H, s), 8.87 (1H, br s).

(2) 9-Fluoro-N-[3-(3-methyl-1H-1,2,4-triazol-1-yl)phenyl]-5,6-dihydrobenzo[h]quinazolin-4-amine, pale yellow crystals.

Mass: 373 (m/z. (M+H)⁺) NMR(DMSO-d₆, δ): 2.38 (3H, s), 2.96 (4H, br s), 7.1-7.6 (4H, m), 7.7-8.0 (2H, m), 8.1-8.3 (1H, m), 8.63 (1H, s), 8.96 (1H, br s), 9.11 (1H, s).

(3) 9-Fluoro-N-[3-(1,2-dimethyl-1H-imidazol-5-yl)phenyl]-5,6-dihydrobenzo[h]quinazolin-4-amine, crystals.

Mass: 386 (m/z. (M+H)⁺) NMR(DMSO-d₆, δ): 2.36 (3H, s), 2.96 (4H, br s), 3.58 (3H, s), 6.88 (1H, s), 7.0-7.5 (4H, m), 7.6-8.0 (3H, m), 8.58 (1H, s), 8.81 (1H, br s).

(4) 9-Fluoro-N-[3-(1,2-dimethyl-1H-imidazol-5-yl)-5-methoxyphenyl]-5,6-dihydrobenzo[h]quinazolin-4-amine, crystals.

Mass: 416 (m/z. (M+H)⁺) NMR(DMSO-d₆, δ): 2.36 (3H, s), 2.95 (4H, br s), 3.59 (3H, s), 3.80(3H, s), 6.67 (1H, br s), 6.89 (1H, s), 7.1-7.6 (4H, m), 7.87 (1H, dd, J=2.7, 10.1 Hz), 8.61 (1H, s), 8.75 (1H, br s).

### EXAMPLE 124

To a solution of 3-[4-({[tert-butyl(dimethyl)silyl]oxy}methyl)-1H-imidazol-1-yl]aniline (500 mg) in tetrahydrofuran (25 ml) was added dropwise with 1.56M solution of n-butyl lithium in n-hexane (1.2 ml) with stirring at 0° C. After stirring for additional 30 minutes at the same temperature, 4-chloro-5,6-dihydrobenzo[h]quinazoline (393 mg) was added to the reaction mixture and the stirring was continued for 2 hours at ambient temperature. After condensation of the reaction mixture under reduced pressure, water (30 ml) was added to the residue and extracted with a mixture of dichloromethane and methanol (20:1) (30 ml×2). The organic extracts were dried over magnesium sulfate and evaporated. The obtained residue was chromatographed on silica gel eluting with a mixture of dichloromethane and methanol (1%, 1.5%, 2% and then 3%). The obtained product was triturated with a mixture of ethyl acetate and n-hexane to give N-{3-[4-({[tert-butyl(dimethyl)silyl]oxy}methyl)-1H-imidazol-1-yl]phenyl}-5,6-dihydrobenzo[h]quinazolin-4-amine (116 mg) as white crystals.

Mass: 484 (m/z. (M+H)⁺) NMR(DMSO-d₆, δ): 0.10 (6H, s), 0.90 (9H, s), 2.8-3.1 (4H, m), 4.62 (2H, br s), 7.2-7.6 (6H, m), 7.7-7.8 (1H, m), 7.9-8.3 (3H, m), 8.61 (1H, s), 8.84(1H, br s).

### EXAMPLE 125

A mixture of N-{3-[4-{[tert-butyl(dimethyl)silyl]oxy}methyl)-1H-imidazol-1-yl]phenyl}-5,6-dihydrobenzo[h]quinazolin-4-amine (110 mg) and a mixture of acetic acid, water and tetrahydrofuran (3:1:1)(2 ml) was stirred for 16 hours at ambient temperature. The reaction mixture was diluted with ethyl acetate (30 ml) and washed with an aqueous saturated solution of sodium hydrogencarbonate (20 ml×2). The organic layer was dried over magnesium sulfate and filtered. After evaporation of the solvent, the residue was triturated with ethyl acetate and chromatographed on silica gel eluting with a mixture of dichloromethane and methanol (1%, 4% and then 8%). The obtained product was triturated with ethyl acetate again to give {1-[3-(5,6-dihydrobenzo[h]quinazolin-4-ylamino)phenyl]-1H-imidazol-4-yl}-methanol (41 mg) as white crystals.

Mass: 370 (m/z. (M+H)⁺) NMR(DMSO-d₆, δ): 2.8-3.1 (4H, m), 4.42 (2H, d, J=5.5 Hz), 4.98 (1H, t, J=5.5 Hz), 7.2-7.6 (6H, m), 7.7-7.8 (1H, m), 7.9-8.3 (3H, m), 8.62 (1H, s), 8.82 (1H, br s).

### EXAMPLE 126

To a suspension of N-[3-(4-methyl-1H-imidazol-1-yl)phenyl]-5,6-dihydrobenzo[h]quinazolin-4-amine (1.0 g) in methanol (10 ml) was added a mixture of 4N-hydrochloric acid and ethyl acetate (0.78 ml) at ambient temperature. After stirring for 10 minutes, diisopropyl ether (20 ml) was added dropwise to the solution and the stirring was continued for 2 hours. The resultant precipitates were collected by filtration, washed with diisopropyl ether and dried under reduced pressure at 50° C. for 4 hours to give N-[3-(4-methyl-1H-imidazol-1-yl)phenyl]-5,6-dihydrobenzo[h]quinazolin-4-amine hydrochloride (1.053 g) as a pale yellow solid.

NMR(DMSO-d₆, δ): 2.37 (3H, br s), 2.99 (4H, br s), 7.3-7.5 (4H, m), 7.58 (1H, t, J=8.1 Hz), 7.8-8.0 (2H, m), 8.1-8.3 (2H, m), 8.65 (1H, s), 9.25 (1H, br s), 9.55 (1H, d, J=1.6 Hz).

### EXAMPLE 127

To a suspension of N-[3-(4-methyl-1H-imidazol-1-yl)phenyl]-5,6-dihydrobenzo[h]quinazolin-4-amine (600 mg) in methanol (10 ml) was added a mixture of 4N-hydrochloric acid and ethyl acetate (0.93 ml) at ambient temperature. After stirring for 10 minutes, diisopropyl ether (20 ml) was added dropwise to the solution and stirring was continued for 2 hours. The resultant precipitates were collected by filtration, washed with a mixture of methanol and diisopropyl ether (1:2) and dried under reduced pressure for 4 hours at 50° C. to give N-[3-(4-methyl-1H-imidazol-1-yl)phenyl]-5,6-dihydrobenzo[h]quinazolin-4-amine dihydrochloride (680 mg) as a pale yellow solid.

NMR(DMSO-d₆, δ): 2.38 (3H, br s), 3.01 (4H, br s), 7.3-7.6 (4H, m), 7.63 (1H, t, J=8.0 Hz), 7.7-7.9 (1H, m), 7.99 (1H, br s), 8.1-8.3 (2H, m), 8.71 (1H, s), 9.5-9.7 (2H, m).

### EXAMPLE 128

To a suspension of N-[3-(4-methyl-1H-imidazol-1-yl)phenyl]-5,6-dihydrobenzo[h]quinazolin-4-amine (1.0 g) in methanol (10 ml) was added methanesulfonic acid (272 mg) at ambient temperature. After stirring for 10 minutes, diisopropyl ether (20 ml) was added dropwise to the solution. The stirring was continued for 2 hours, and the resultant precipitates were collected by filtration. The precipitates were washed with a mixture of methanol and diisopropyl ether (1:2) and dried under reduced pressure for 4 hours at 60° C. to give N-[3-(4-methyl-1H-imidazol-1-yl)phenyl]-5,6-dihydrobenzo[h]quinazolin-4-amine methanesulfonate (1.15 g) as a white solid.

NMR(DMSO-d₆, δ): 2.37 (6H, br s), 2.99 (4H, br s), 7.3-7.5 (4H, m), 7.60 (1H, t, J=8.1 Hz), 7.8-7.9 (1H, m), 8.0 (1H, br s), 8.1-8.3 (2H, m), 8.67 (1H, s), 9.25 (1H, br s), 9.57 (1H, d, J=1.6 Hz).

### EXAMPLE 129

To a suspension of N-[3-(4-methyl-1H-imidazol-1-yl)phenyl]-5,6-dihydrobenzo[h]quinazolin-4-amine (197.6 g) in methanol (6 L) was added dropwise (30 min) methanesulfonic acid (113 g) at 5-10° C. After stirring for 4 hours at ambient temperature, the resultant suspension was added with methanol (3.7 L) and heated at reflux. The resultant solution was filtered and washed with methanol. The mixture was allowed to stand for overnight at ambient temperature and concentrated to about 2L under reduced pressure. The suspension was stirred at ambient temperature for 2 hours, and the precipitates were collected by filtration. The precipitates were washed with methanol (200 ml×3) and dried under reduced pressure for 4 hours at 50° C. to give N-[3-(4-methyl-1H-imidazol-1-yl)phenyl]-5,6-dihydrobenzo[h]quinazolin-4-amine dimethanesulfonate (287.7 g) as pale yellow crystals.

NMR(D₂O, δ): 2.42 (3H, br s), 2.81 (6H, s), 2.7-3.1 (4H, m), 7.3-7.8 (9H, m), 8.59 (1H, s), 8.98 (1H, d J=1.6 Hz).

1. A compound of the formula (I): 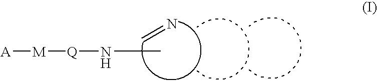
wherein A is a hydrogen atom, an optionally substituted, unsaturated, N-containing heterocyclic group or a group of the formula (a): 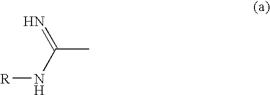
wherein R is an optionally substituted aryl group or an optionally substituted heterocyclic group;
M is —(CH₂)ₙ—, —(CH₂)ₙ—O—(CH₂)ₘ— or —(CH₂)ₙ—NH—(CH₂)ₘ—, wherein n and m are independently 0, 1 or 2;
Q is an optionally substituted cycloalkylene group, an optionally substituted arylene group or an optionally substituted, divalent heterocyclic group; and
the moiety of the formula (b): 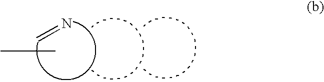
is an optionally substituted, unsaturated, mono-, di-, tri- or tetra-cyclic, N-containing heterocyclic group which may contain additional hetero atom(s) selected from the group consisting of nitrogen, oxygen and sulfur atoms as the ring member(s),
its prodrug or a pharmaceutically acceptable salt thereof.

2. The compound of claim 1, wherein
the heterocyclic moiety in the optionally substituted, unsaturated N-containing heterocyclic group for A is an unsaturated, 5- to 10-membered, mono- or di-cyclic, N-containing heterocyclic group;
the heterocyclic moiety in the optionally substituted heterocyclic group for R is a 5- to 6-membered S-containing heterocyclic group;
the heterocyclic moiety in the optionally substituted, divalent heterocyclic group for Q is a 6-membered divalent N-containing heterocyclic group;
the mono-, di-, tri or tetra-cyclic moiety in the optionally substituted, unsaturated, mono-, di-, tri or tetra-cyclic, N-containing heterocyclic group for the moiety of the formula (b) is
an unsaturated, N— or N and S-containing, 5- to 6-membered, monocyclic group,
an unsaturated, N— or N and O— or N and S-containing 9- to 10-membered, di-cyclic group,
an unsaturated, N— or N and O— or N and S-containing, 12- to 15-membered, tri-cyclic group, or
an unsaturated, N-containing, 16-membered, tetra-cyclic group.

3. The compound of claim 2, wherein
the heterocyclic moiety for A is one containing 1 to 4 nitrogen atoms;
the heterocyclic moiety for R is one containing one sulfur atom;
the heterocyclic moiety for Q is one containing 1 to 2 nitrogen atoms;
the mono-cyclic, heterocyclic moiety represented by the formula (b) is one containing 1 to 2 nitrogen atoms or 1 to 2 nitrogen atoms and one sulfur atom;
the di-cyclic, heterocyclic moiety represented by the formula (b) is one containing 1 to 3 nitrogen atoms or 1 to 2 nitrogen atoms and one oxygen atom or 1 to 2 nitrogen atoms and one sulfur atom;
the tri-cyclic, heterocyclic moiety represented by the formula (b) is one containing 1 to 4 nitrogen atoms or 1 to 3 nitrogen atoms and 1 to 2 oxygen atoms or 1 to 3 nitrogen atoms and 1 to 2 sulfur atoms; and
the tetra-cyclic, heterocyclic moiety represented by the formula (b) is one containing 1 to 3 nitrogen atoms.

4. The compound of claim 3, wherein
the heterocyclic moiety for A is imidazolyl, triazolyl, pyridyl, pyrimidinyl, benzimidazolyl or isoquinolyl;
the heterocyclic moiety for R is thienyl;
the heterocyclic moiety for Q is pyridinediyl or pyrimidinediyl;
the mono-cyclic, heterocyclic moiety of the formula (b) is thiazolyl, pyridyl, pyridazinyl or pyrimidinyl;
the di-cyclic, heterocyclic moiety of the formula (b) is isoquinolyl, phthalazinyl, quinazolinyl, benzothiazolyl, benzisoxazolyl, benzimidazolyl, imidazo[1,5-a]pyridyl or 6,7,8,9-tetrahydro-5H-cyclohepta[d]pyrimidinyl;
the tri-cyclic, heterocyclic moiety of the formula (b) is 5,6-dihydrobenzo[h]quinazolinyl, 4,5-dihydro[1]benzoxepino[5,4-c]isoxazolyl, 9H-indeno[2,1-d]pyrimidinyl, 5,6-dihydro[1]benzoxepino[5,4-d]pyrimidinyl, 5,6-dihydrothieno[2,3-h]quinazolinyl, 4,5-dihydronaptho[2,1-d]thiazolyl or 3H-indeno[2,1-d]thiazolyl; and
the tetra-cyclic, heterocyclic moiety of the formula (b) is indeno[1,2,3-de]phthalazinyl.

5. The compound of any one of claims 1 to 4, wherein
the substituent(s) on the heterocyclic group for A is(are) lower alkyl and/or hydroxy(lower)alkyl;
the substituent(s) on the aryl group or heterocyclic group for R is (are) halogen;
the substituent(s) on the cycloalkylene, arylene or divalent heterocyclic group for Q is (are) halogen, lower alkyl, lower alkoxy and/or halo(lower)alkyl;
the substituent(s) on the mono-, di-, tri- or tetra-cyclic, heterocyclic group for the moiety of the formula (b) is(are) halogen, lower alkyl, lower alkoxy, halo(lower)alkyl, pyrrolyl, thienyl, anilino, phenoxy and/or phenyl, among which the phenyl may be further substituted with halogen, hydroxy, lower alkyl and/or lower alkoxy.

6. The compound of any one of claims 1 to 5, wherein
A is an optionally substituted, unsaturated, 5-membered, N-containing heterocyclic group,
M is a group of —(CH₂)ₙ— in which n is 0,
Q is an optionally substituted arylene group, and
the moiety of the formula (b) is an optionally substituted, unsaturated, tricyclic heterocyclic group containing 2 nitrogen atoms.

7. The compound of claim 6, wherein
A is an unsaturated, 5-membered, N-containing heterocyclic group substituted with lower alkyl and
Q is arylene group.

8. The compound of claim 7, wherein
A is an imidazolyl group substituted with one or two lower alkyl,
Q is phenylene group, and
the group of formula (b) is a 5,6-dihydrobenzo[h]quinazolinyl group which may be substituted with a halogen atom.

9. A compound of claim 8, which is selected from the groups consisting of
N-[3-(4,5-dimethyl-1H-imidazol-1-yl)phenyl]-9-fluoro-5,6-dihydrobenzo[h]quinazolin-4-amine,
9-Fluoro-N-[3-(4-methyl-1H-imidazol-1-yl)phenyl]-5,6-dihydrobenzo[h]quinazolin-4-amine,
9-Fluoro-N-[3-(1,2-dimethyl-1H-imidazol-5-yl)phenyl]-5,6-dihydrobenzo[h]quinazolin-4-amine,
N-[3-(4-methyl-1H-imidazol-1-yl)phenyl]-5,6-dihydrobenzo[h]quinazolin-4-amine hydrochloride,
N-[3-(4-methyl-1H-imidazol-1-yl)phenyl]-5,6-dihydrobenzo[h]quinazolin-4-amine dihydrochloride,
N-[3-(4-methyl-1H-imidazol-1-yl)phenyl]-5,6-dihydrobenzo[h]quinazolin-4-amine methanesulfonate,
N-[3-(4-methyl-1H-imidazol-1-yl)phenyl]-5,6-dihydrobenzo[h]quinazolin-4-amine dimethanesulfonate,
N-[3-(1,2-dimethyl-1H-imidazol-5-yl)phenyl]-5,6-dihydrobenzo[h]quinazolin-4-amine,
N-[3-(4,5-dimethyl-1H-imidazol-1-yl)phenyl]-5,6-dihydrobenzo[h]quinazolin-4-amine and
N-[3-(4-methyl-1H-imidazol-1-yl)phenyl]-5,6-dihydrobenzo[h]quinazolin-4-amine.

10. A pharmaceutical composition comprising an effect amount of a compound of the formula (I) of claim 1, its prodrug or a pharmaceutically acceptable salt thereof, as an active ingredient, in admixture with a pharmaceutically acceptable carrier or excipient.

11. The pharmaceutical composition of claim 10 for the use of treatment and/or prevention of anxiety, depression, obsessive compulsive disorders, migraine, anorexia, Alzheimer's disease, sleep disorders, bulimia, panic attacks, withdrawal from drug abuse, schizophrenia, and disorders associated with spinal trauma and/or head injury.

12. A use of the compound of claim 1 for the manufacture of a medicament for treatment and/or prevention of anxiety, depression, obsessive compulsive disorders, migraine, anorexia, Alzheimer's disease, sleep disorders, bulimia, panic attacks, withdrawal from drug abuse, schizophrenia, and disorders associated with spinal trauma and/or head injury.

13. A method for the use of the treatment and/or prevention of anxiety, depression, obsessive compulsive disorders, migraine, anorexia, Alzheimer's disease, sleep disorders, bulimia, panic attacks, withdrawal from drug abuse, schizophrenia, and disorders associated with spinal trauma and/or head injury by administering the compound of claim 1.
